(19) United States
(12) Patent Application Publication
(10) Pub. No.: US 2023/0159661 A1
(43) Pub. Date: May 25, 2023

(54) T CELL REDIRECTING BISPECIFIC ANTIBODIES FOR THE TREATMENT OF EGFR POSITIVE CANCERS

(71) Applicant: Ichnos Sciences SA, La Chaux-de-Fonds (CH)

(72) Inventors: Rami LISSILAA, La Chaux-de-Fonds (CH); Cian STUTZ, La Chaux-de-Fonds (CH); Stanislas BLEIN, Epalinges (CH)

(73) Assignee: Ichnos Sciences SA, La Chaux-de-Fonds (CH)

(21) Appl. No.: 16/607,783

(22) PCT Filed: Apr. 24, 2018
(86) PCT No.: PCT/EP2018/060488

§ 371 Date: Oct. 24, 2019

(30) Foreign Application Priority Data
Apr. 24, 2017 (EP) 17167709.9

(51) Int. Cl. C07K 16/46; A61P 35/00

(52) U.S. Cl. CPC C07K 16/468; A61P 35/00; C07K 2317/31; C07K 2317/622; C07K 2317/55

(57) ABSTRACT

The present invention relates to bispecific antibodies which bind to CD3 and EGFR simultaneously. This class of antibody has been demonstrated by the inventors to be useful in the treatment of EGFR tumors by redirecting T cells and forming an immune synapse between activated T cells and EGFR expressing tumor cells, leading to increased levels of killing of EGFR expressing tumor cells. In particular the present invention relates to CD3×EGFR bispecific antibodies selected from the group comprising CD3×EGFR_SF1 (SEQ ID NO: 4, 5 and 6), CD3×EGFR_SF3 (SEQ ID NO: 7, 2 and 8), CD3×EGFR_SF4 (SEQ ID NO: 4, 5 and 9), CD3×EGFR_SD1 (SEQ ID 10 NO: 1, 2 and 10) and CD3×EGFR_SD2 (SEQ ID NO: 11, 10 and 2).

### REFERENCE TO A SEQUENCE LISTING

This application includes a Sequence Listing entitled 3305_0270001_Seqlisting_ST25.txt, generated on Jun. 8, 2020 with a size of 136,982 bytes. The Sequence Listing is incorporated by reference.

### TECHNICAL FIELD

The present invention relates to bispecific antibodies which bind to CD3 and EGFR simultaneously. This class of antibody has been demonstrated by the inventors to be useful in the treatment of EGFR tumors by redirecting T cells and forming an immune synapse between activated T cells and EGFR expressing tumor cells, leading to increased levels of killing of EGFR expressing tumor cells.

### BACKGROUND OF INVENTION

Targeting epidermal growth factor receptor (EGFR) overexpressed by many epithelial-derived cancer cells with anti-EGFR monoclonal antibodies (mAb) has been demonstrated to inhibit their growth, leading to positive clinical outcomes.

Cancer immunotherapy or immune-oncology is as the fourth antitumor modality and has undergone a period of growth following in some cases encouraging and in others remarkable data regarding its clinical efficacy.

Clinical responses in patients treated with an anti-EGFR mAb, have been variable however and may reflect variability in EGFR expression, signaling in neoplastic cells, adaptive mechanisms used by cancer cells to evade therapy or likely some combination of all these factors.

One well elucidated mechanism by which cancer cells can become resistant to anti-EGFR mAb therapy, is by mutation of the Kirsten ras (KRAS) oncogene homolog from the mammalian ras gene family. Somatic KRAS mutations are found at high rates in leukemias, colorectal cancer, pancreatic cancer and lung cancer. KRAS mutation is predictive of a very poor response to the approved anti-EGFR mAb therapies panitumumab (Vectibix®) and cetuximab (Erbitux®) in colorectal cancer. Studies show patients whose tumors express the mutated version of the KRAS gene will not respond to cetuximab or panitumumab. The emergence of KRAS mutations is a frequent driver of acquired resistance to anti-EGFR mAb therapies in colorectal and other cancers.

### SUMMARY OF THE INVENTION

To address the problems associated with the treatment of EGFR cancers, the inventors have generated a new set of anti-tumor medicaments which are suitable for treating EGFR overexpressing cancers and overcome the problems of existing therapies.

The present invention relates to a bispecific antibody which binds to epitopes upon CD3ε and EGFR.

Wherein the CD3ε binder is preferably SP34 or OKT3 or derived therefrom.

Wherein the EGFR binder is preferably panitumumab and cetuximab.

In accordance with the present invention the CD3×EGFR bispecific antibody comprises at least one FAB and one scFv binding portion.

In particular the present invention relates to binding portions from protein based target specific binding molecules such as antibodies, DARPins, Fynomers, Affimers, variable lymphocyte receptors, anticalin, nanofitin, variable new antigen receptor (VNAR), but is not limited to these.

In particular the binding portions are taken or derived from an antibody such as a Fab, Fab′, Fab′-SH, Fd, Fv, dAb, F(ab′)2, scFv, Fcabs, bispecific single chain Fv dimers, diabodies, triabodies. In preferred embodiments the agonist comprises binding portions taken or derived from Fab, ScFv and dAb.

In accordance with the present invention the CD3×EGFR bispecific antibody comprises at least one FAB and one scFv portion concatenated to each other.

In particular the binding portions maybe genetically fused to a scaffold comprising the same or a different antibody Fc or a portion thereof. In accordance with this aspect of the present invention, a first full length antibody such as an IgG may form the basis of a CD3×EGFR bispecific antibody according to the present invention and a second set of binding portions may be grafted onto the starting antibody in accordance with the present invention.

Preferably the two binding portions are concatenated such that the second binding portion is located distally to the variable portion of the immunoglobulin heavy chain.

Alternatively the two binding portions are concatenated such that the second binding portion is located proximal to the variable portion of the immunoglobulin heavy chain.

Preferably the two binding portions are concatenated such that the second binding portion is located distally to the variable portion of the immunoglobulin light chain.

Alternatively the two binding portions are concatenated such that the second binding portion is located proximal to the variable portion of the immunoglobulin light chain.

In accordance with the present invention the two concatenated binding portions may be separated by a peptide linker.

In accordance with the present invention the CD3×EGFR bispecific antibody is selected from the group comprising CD3×EGFR_SF1 (SEQ ID NOs: 4, 5 and 6), CD3×EGFR_SF3 (SEQ ID NOs: 7, 2 and 8), CD3×EGFR_SF4 (SEQ ID NOs: 4, 5 and 9), CD3×EGFR_SD1 (SEQ ID NOs: 1, 2 and 10) and CD3×EGFR_SD2 (SEQ ID NOs: 11, 10 and 2).

In accordance with another aspect of the present invention relates to an antibody or fragment thereof that binds to domain 4 of human EGFR and which comprises a heavy and light variable sequence selected from the group: SEQ ID NOs: 23 and 24, SEQ ID NOs: 25 and 26, SEQ ID NOs: 31 and 33, SEQ ID NOs: 32 and 34, SEQ ID NOs: 36 and 38, SEQ ID NOs: 37 and 39 or derived therefrom.

The present invention also relates to the use of the CD3×EGFR bispecific antibody according to the present invention as a medicament.

The present invention also relates to the use of CD3×EGFR bispecific antibody according to the present invention as a medicament for the treatment of cancer or other disease characterised or exacerbated by over expression of EGFR.

The present invention also relates to a method of treating a patient suffering from cancer, involving administering to the patient an effective amount of the CD3×EGFR bispecific antibody.

The present invention also relates to a method of treating a patient suffering from cancer, involving administering to the patient an effective amount of the CD3×EGFR bispecific antibody and one or more other agents, such as small molecule or biological medicines to further modulate the immune system of the patient. Examples of such agents include anti-PD-1 antibodies and antineoplastic small molecules such as multikinase inhibitors.

Further the present invention relates to the co-administration of the CD3×EGFR bispecific antibody according to the present invention and another medicament to a patient, wherein the other medicament has a synergistic or additive effect.

In accordance with another aspect of the present invention there is provided a method of treating an EGFR expressing cancer by administering a therapeutic amount of a CD3×EGFR bispecific antibody according to the present invention to a patient in need.

In accordance with another aspect of the present invention there is provided a CD3×EGFR bispecific antibody according to the present invention for use as a medicament.

In accordance with another aspect of the present invention there is provided a CD3×EGFR bispecific antibody according to the present invention for use as a treatment of EGFR expressing cancer.

In accordance with another aspect of the present invention the EGFR expressing cancer further comprises provided a one or more KRAS or B-Raf mutation.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses (also known as isotypes) as well, such as IgG1, IgG2, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

The single domain antibody (sdAb) fragments portions of the fusion proteins of the present disclosure are referred to interchangeably herein as targeting polypeptides herein.

As used herein, the term “epitope” includes any protein determinant capable of specific binding to/by an immunoglobulin or fragment thereof, or a T-cell receptor. The term “epitope” includes any protein determinant capable of specific binding to/by an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 mM, for example, in some embodiments, ≤1 μM; e.g., ≤100 nM, ≤10 nM or ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (KD) of the interaction, wherein a smaller KD represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (kon) and the “off rate constant” (koff) can be determined by calculation of the concentrations and the actual rates of association and dissociation (See Nature 361:186-87 (1993)). The ratio of koff/kon enables the cancellation of all parameters not related to affinity, and is equal to the dissociation constant KD (See, generally, Davies et al. (1990) Annual Rev Biochem 59:439-473). An antibody of the present disclosure is said to specifically bind to an antigen, when the equilibrium binding constant (KD) is ≤1 mM, in some embodiments ≤1 μM, ≤100 nM, ≤10 nM, or ≤100 pM to about 1 pM, as measured by assays such as radioligand binding assays, surface plasmon resonance (SPR), flow cytometry binding assay, or similar assays known to those skilled in the art.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, for example, at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, for example, at least 90 percent sequence identity, at least 95 percent sequence identity, or at least 99 percent sequence identity.

In some embodiments, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Suitable conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, for example, at least 80%, 90%, 95%, or 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Suitable amino- and carboxy-termini of fragments or analogs occur near boundaries of functional domains. Structural and functional domains can be identified by comparison of the nucleotide and/or amino acid sequence data to public or proprietary sequence databases. In some embodiments, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Suitable amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (for example, conservative amino acid substitutions) may be made in the naturally-occurring sequence (for example, in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, for example, at least 14 amino acids long, at least 20 amino acids long, at least 50 amino acids long, or at least 70 amino acids long. The term “analog” as used herein refers to polypeptides which are comprised of a segment of at least 25 amino acids that has substantial identity to a portion of a deduced amino acid sequence and which has specific binding to CD47, under suitable binding conditions. Typically, polypeptide analogs comprise a conservative amino acid substitution (or addition or deletion) with respect to the naturally-occurring sequence. Analogs typically are at least 20 amino acids long, for example, at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH2NH—, —CH2S—, —CH2-CH2--, —CH═CH--(cis and trans), —COCH2--, CH(OH)CH2--, and —CH2SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, and/or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, the terms “treat,” treating,” “treatment,” and the like refer to reducing and/or ameliorating a disorder and/or symptoms associated therewith. By “alleviate” and/or “alleviating” is meant decrease, suppress, attenuate, diminish, arrest, and/or stabilize the development or progression of a disease such as, for example, a cancer. It will be appreciated that, although not precluded, treating a disorder or condition does not require that the disorder, condition or symptoms associated therewith be completely eliminated.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and in some embodiments, a substantially purified fraction is a composition wherein the object species comprises at least about 50 percent (on a molar basis) of all macromolecular species present.

Generally, a substantially pure composition will comprise more than about 80 percent of all macromolecular species present in the composition, for example, more than about 85%, 90%, 95%, and 99%. In some embodiments, the object species is purified to essential homogeneity (contaminant species cannot be detected in the composition by conventional detection methods) wherein the composition consists essentially of a single macromolecular species.

In this disclosure, “comprises,” “comprising,” “containing,” “having,” and the like can have the meaning ascribed to them in U.S. and/or European Patent law and can mean “includes,” “including,” and the like; the terms “consisting essentially of” or “consists essentially” likewise have the meaning ascribed in U.S. patent law and these terms are open-ended, allowing for the presence of more than that which is recited so long as basic or novel characteristics of that which is recited are not changed by the presence of more than that which is recited, but excludes prior art embodiments.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “subject” is meant a mammal, including, but not limited to, a human or non-human mammal, such as a bovine, equine, canine, rodent, ovine, primate, camelid, or feline.

The term “administering,” as used herein, refers to any mode of transferring, delivering, introducing, or transporting a therapeutic agent to a subject in need of treatment with such an agent. Such modes include, but are not limited to, oral, topical, intravenous, intraperitoneal, intramuscular, intradermal, intranasal, and subcutaneous administration.

There follows a brief summary of the figures.

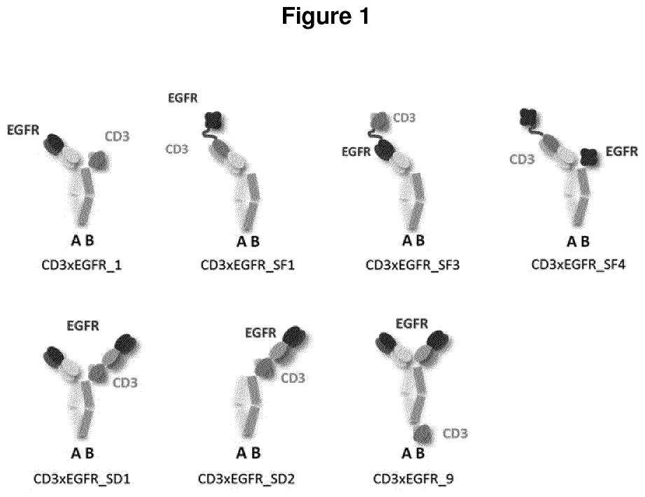

FIG. 1: The panitumumab anti-EGFR binder (black) and the humanized SP34 anti-CD3 binder (grey) where assembled in various different BEAT architectures.

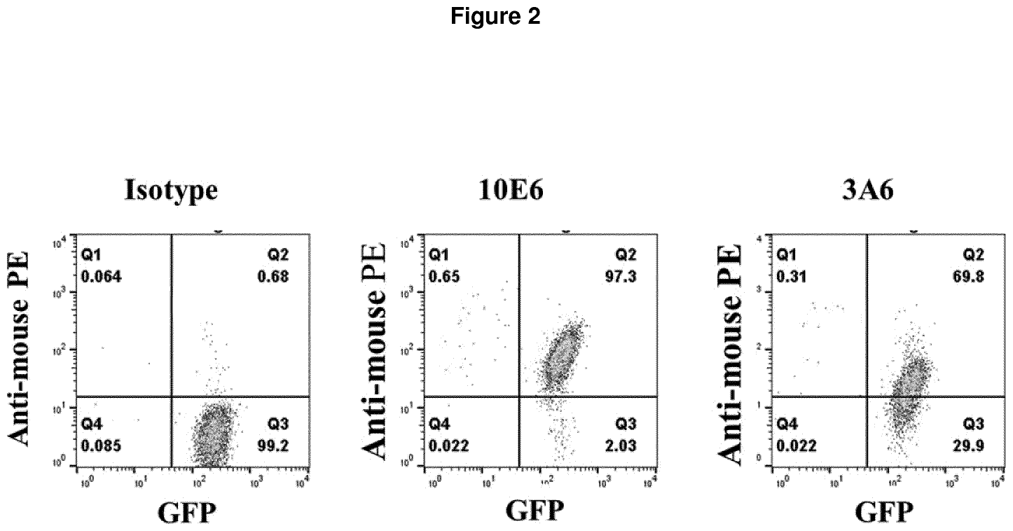

FIG. 2: Flow cytometry analysis of 3A6 and 10E6 hybridoma candidates on BAF cells expressing membrane-bound EGFR. This figure shows the FACS profiles of parental 3A6 and 10E6 hybridoma supernatants to membrane-bound EGFR expressed on BAF cells. One hundred μl harvested from both hybridoma clones were incubated with 100 ul of EGFR-transfected BAF cells diluted at 106 cells/ml. As a negative control, a purified mouse IgG isotype was used diluted at 10 μg/ml. Antibody binding was detected with goat anti-mouse IgG-PE.

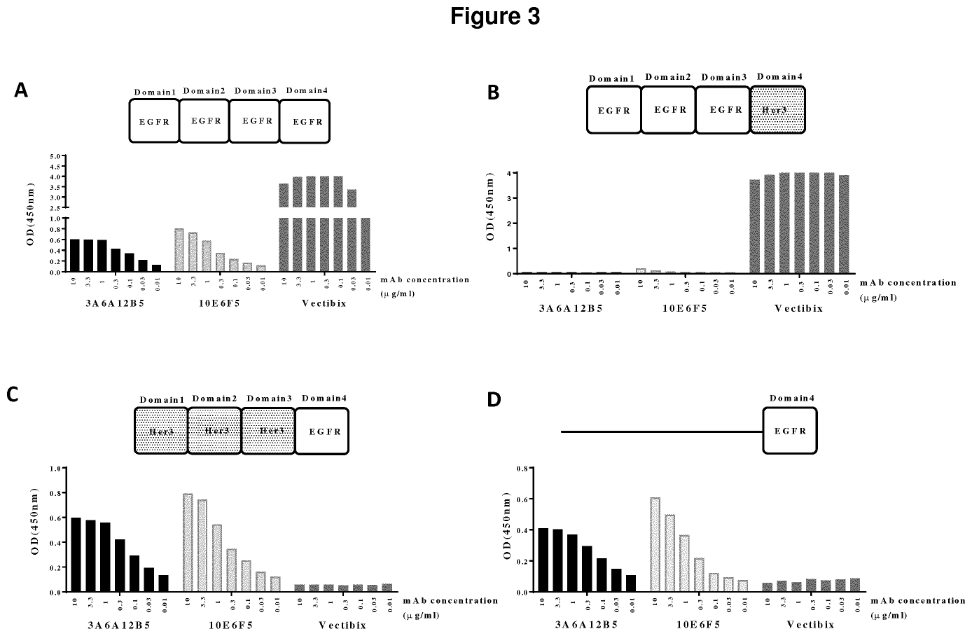

FIG. 3: 3A6A12B5 and 10E6F5 bind specifically to extracellular domain IV of the EGFR receptor. This figure shows the ELISA results in which several concentrations (ranging from 10 to 0.01 μg/ml) of purified 3A6A12F5 and 10E6F5 hybridoma subclones were tested against immobilized recombinant soluble EGFR (A) or EGFR-Her3 chimeric molecules (B and C) or single domain IV of EGFR (D). Vectibix® was also tested in the assay.

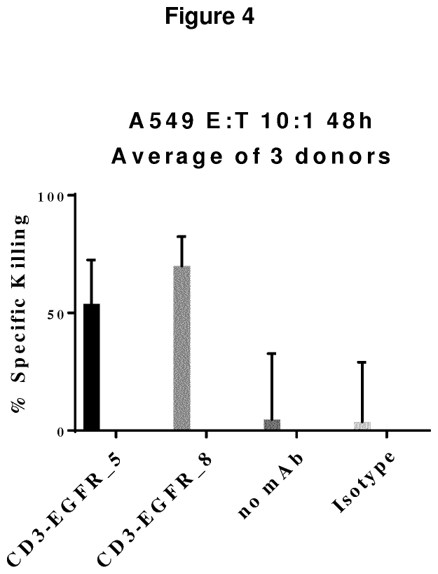

FIG. 4: CD3-EGFR_5 and CD3-EGFR_8 display a killing activity of EGFR+A549 target cells. A CD3-redirected killing assay against EGFR+A549 cells (Target cells, T) was performed using PBMCs from 3 healthy donors as effector cells (E), at an E:T ratio of 10:1, during 48 hours. The histograms show the average percentage of specific killing calculated from the 3 individual donors. The two BEAT molecules were used at 10 nM in the assay.

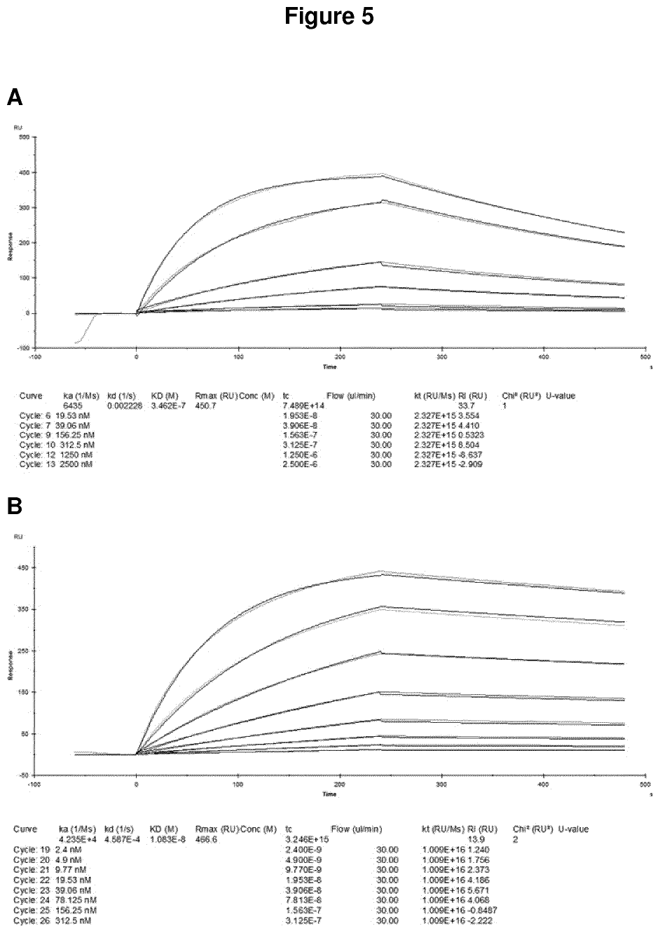

FIG. 5: (A) KD measurement for the chimeric 3A6 antibody. (B) KD measurement for the chimeric 10E6 antibody.

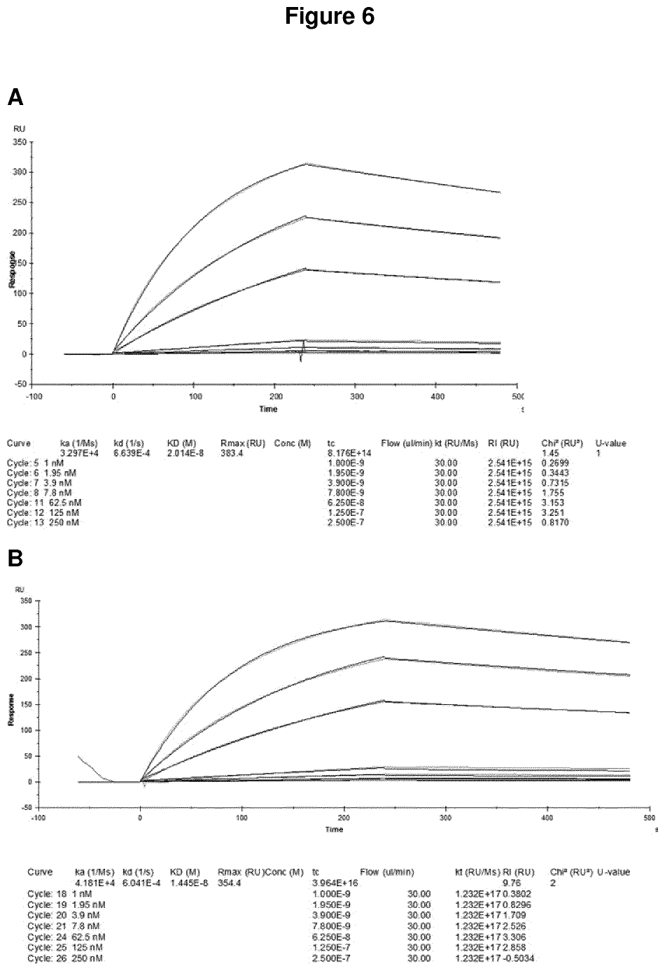

FIG. 6: (A) KD measurement for the 10E6-best-fit antibody. (B) KD measurement for the 10E6-stable antibody.

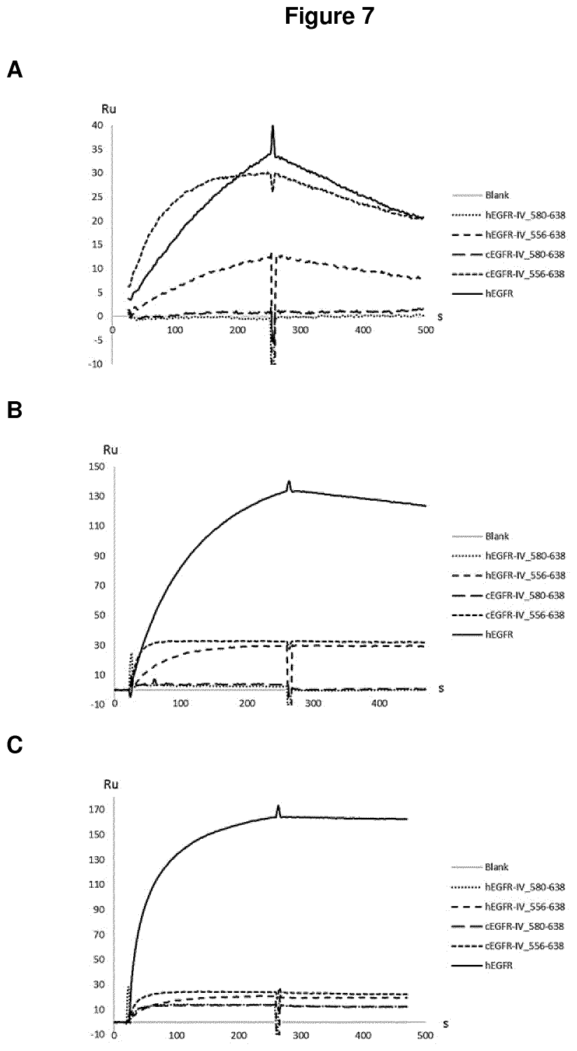

FIG. 7: (A) Sensorgrams of binding tests with 3A6 chimeric antibody, (B) Sensorgrams of binding tests with 10E6 chimeric antibody. (C) Sensorgrams of the control experiment using the polyclonal goat anti-EGFR antibody.

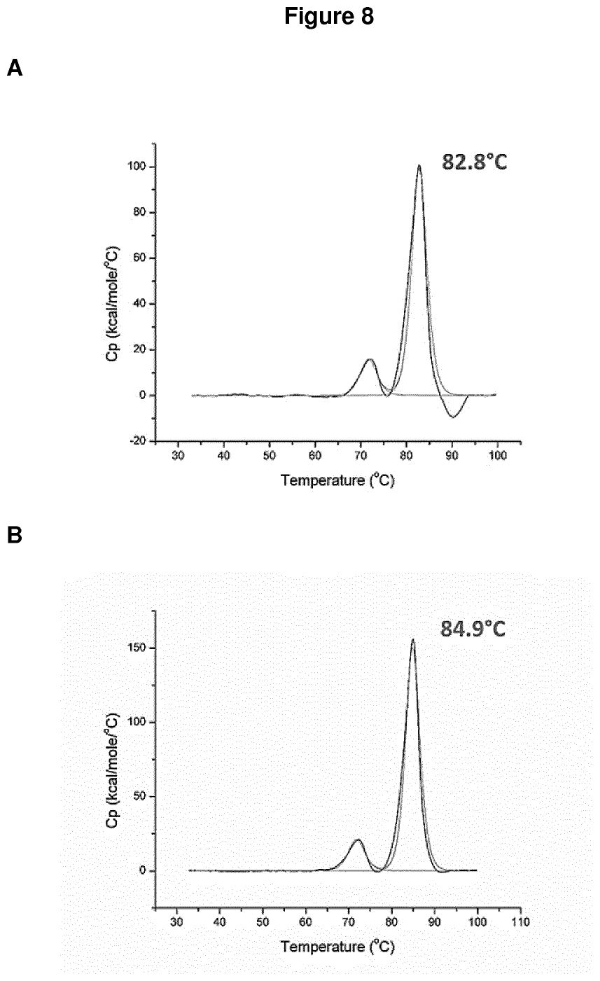

FIG. 8: (A) Thermogram for 10E6-best-fit antibody. The first peak corresponds to the IgG1 CH2-CH3 domains and shows a Tm of 71.7° C., the second peak corresponds to the Fab. (B) Thermogram for 10E6-stable antibody. The first peak corresponds to the IgG1 CH2-CH3 domains and shows a Tm of 71.8° C., the second peak corresponds to the Fab.

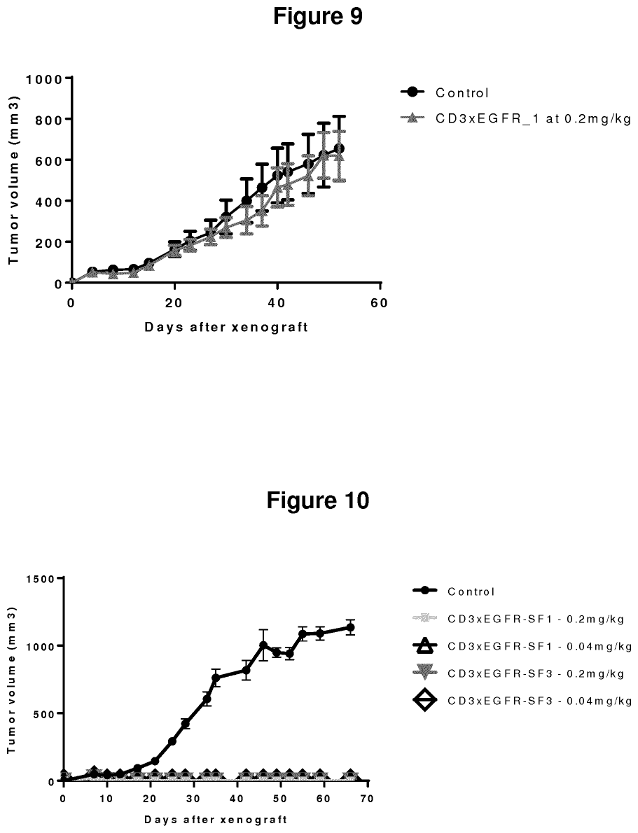

FIG. 9: CD3×EGFR_1 has no efficacy in A549 tumors.

FIG. 10: CD3×EGFR-SF1 and CD3×EGFR-SF3 have the same efficacy in A549 tumors.

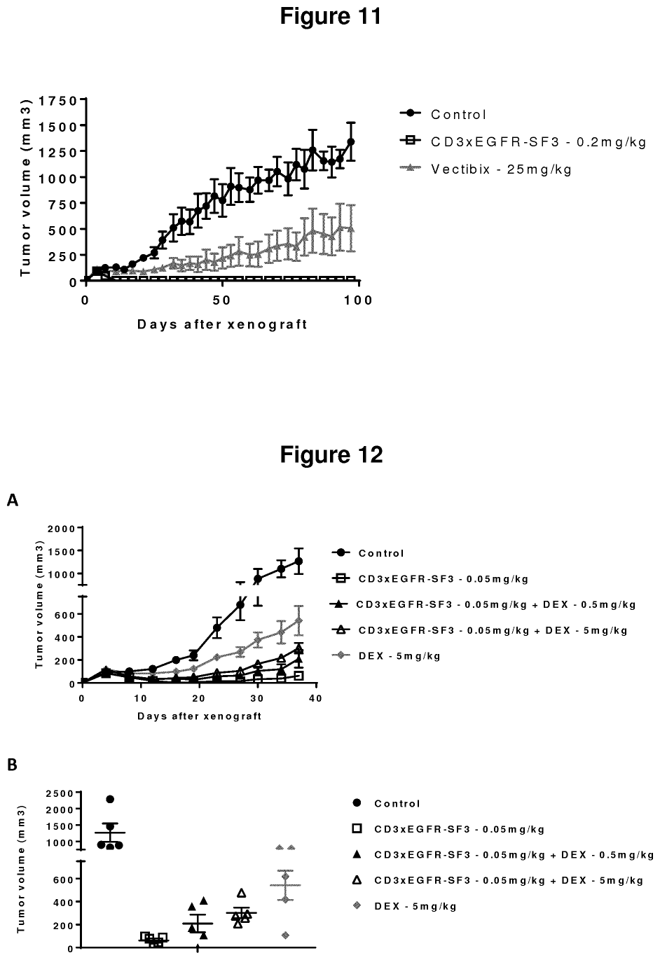

FIG. 11: CD3×EGFR-SF3 displays a better potency than Vectibix in SNU-216 tumors.

FIG. 12: Dexamethasone impact on CD3×EGFR-SF3 anti-tumor activity in xenograft models. (A) The graph shows the mean tumor size (in mm³)±SEM. (B) The graph shows the tumor growth per mouse at day 37.

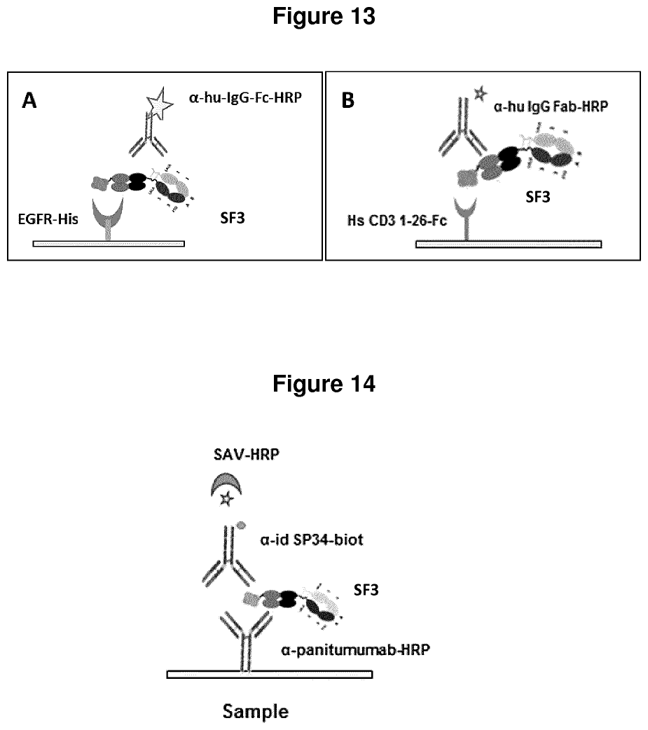

FIG. 13: Simple binding ELISA format schematic for EGFR (A) and CD3 (B).

FIG. 14: Dual binding ELISA format schematic.

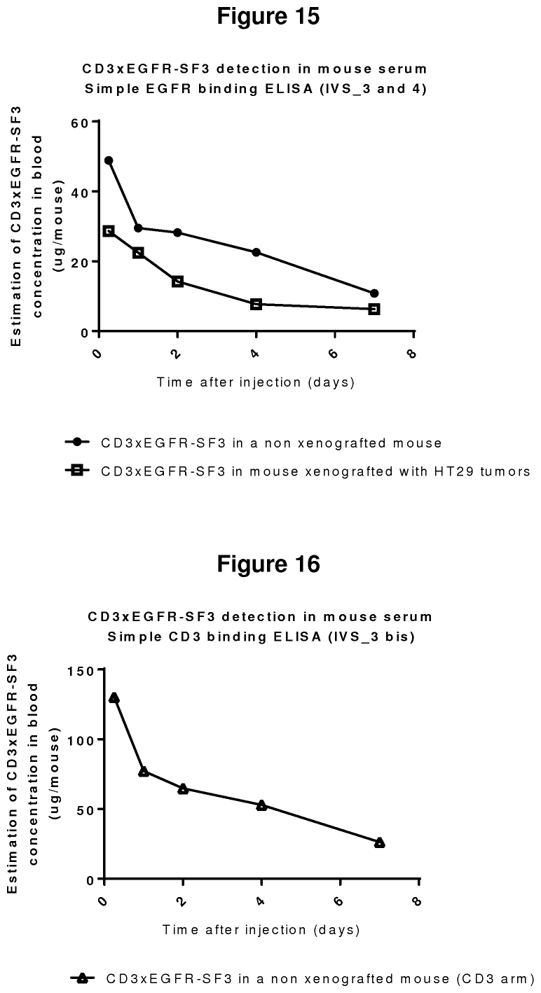

FIG. 15: Detection of CD3×EGFR-SF3 in mice serum by a simple EGFR binding ELISA.

FIG. 16: Detection of CD3×EGFR-SF3 in mice serum by a simple CD3 binding ELISA.

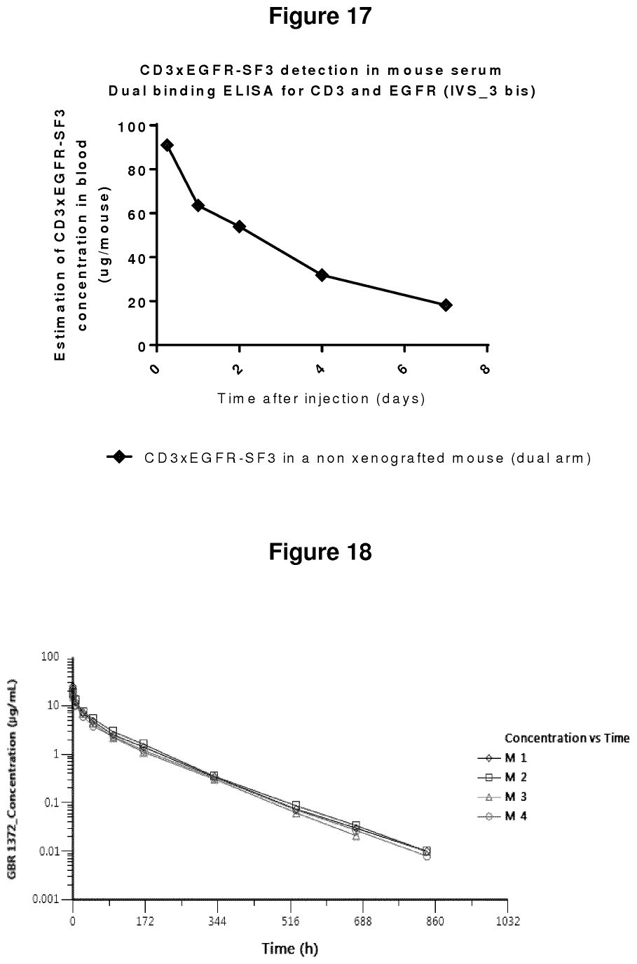

FIG. 17: Detection of CD3×EGFR-SF3 in mice serum by a dual CD3 and EGFR binding ELISA.

FIG. 18: Pharmacokinetic profile of CD3×EGFR-SF3 in Sprague-Dawley rats serum. The pharmacokinetics of CD3×EGFR-SF3 was evaluated in male Sprague-Dawley rats (n=4) following a single intravenous injection at a dose of 1 mg/kg body weight. The blood samples for pharmacokinetic (PK) assessment were collected at pre-specified time points of 0.25, 1, 6, 24, 48, 96, 168, 336, 530, 672, 840 and 1008 hours post dose over a period of 42 days (six weeks). The concentrations of CD3×EGFR-SF3 in these serum samples were quantified using a suitable ELISA method. Data representative of four animals tested (N=1).

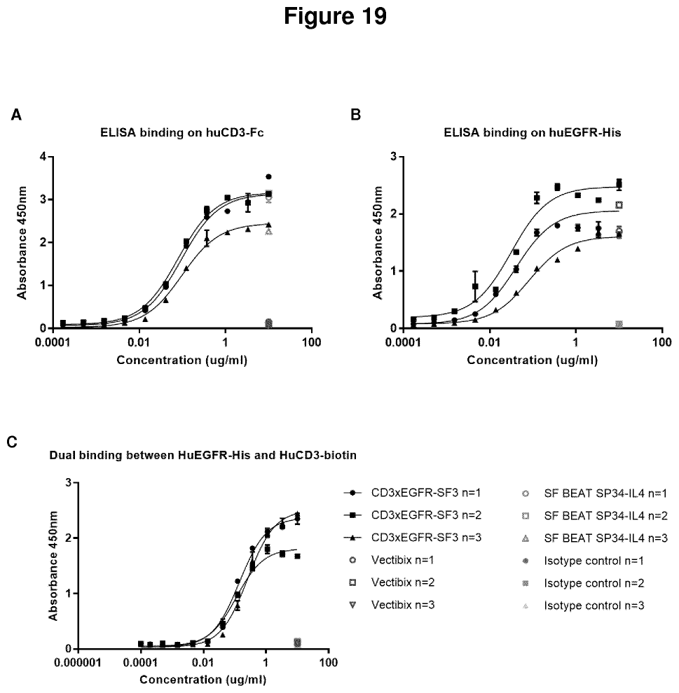

FIG. 19. Detection of CD3×EGFR-SF3 binding by ELISA. A dose response of CD3×EGFR-SF3 and control antibodies were incubated on coated human CD3-Fc (huCD3-Fc, A), human EGFR domain I-IV his-tagged (huEGFR-His; B) or huEGFR-His (C), then detected with either an anti-human IgG Fab coupled with HRP (A and B) or huCD3-biotin followed by HRP-coupled streptavidin (C). The graphs show the sigmoidal dose-response binding curves (absorbance at 450 nM) for each treatment. Each data point is the mean±SEM of duplicates values from three independent replications.

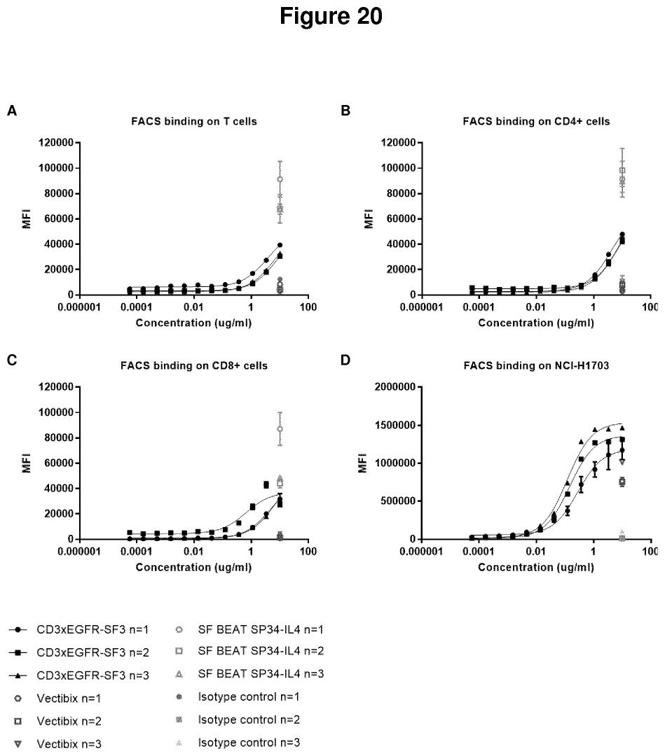

FIG. 20. Detection of CD3×EGFR-SF3 binding by flow cytometry. A dose response of CD3×EGFR-SF3 and control antibodies were incubated on either PBMCS (A-C) or the squamous cancer cell line NCI-H1703 (D) and detected with a PE-labelled anti-human IgG (Fc-γ). For the PBMCs, the cells were also labelled with anti-CD4 or anti-CD8 antibodies. The graphs show the nonlinear sigmoidal regression binding curves of the mean fluorescent intensity (MFI) for each treatment. Each data point is the mean±SEM of duplicates values from three independent replications.

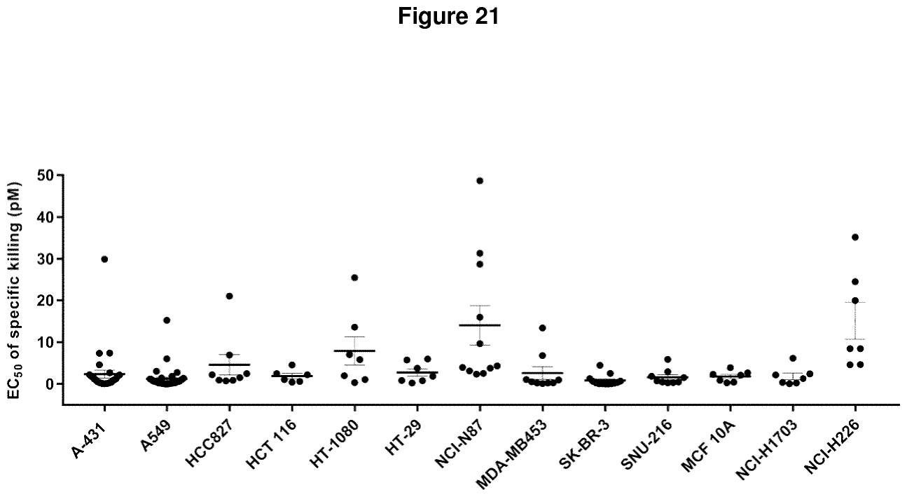

FIG. 21. CD3×EGFR-SF3 induces the redirected lysis of EGFR-expressing human cancer cell lines. Target cancer cells (T) and effector cells (E; PBMCs) were incubated at an E:T ratio of 1:10 in the presence of a dose response of CD3×EGFR-SF3 or control antibodies and the redirected lysis of the cancer cells was determined by a cytotoxic assay (MTS). The EC₅₀ values were extracted from the sigmoidal dose-response curves of specific killing. The error bars represent the mean±SEM. Cell lines redirected lysis was statistically different (one-way ANOVA; F=5,6; p<0.0001).

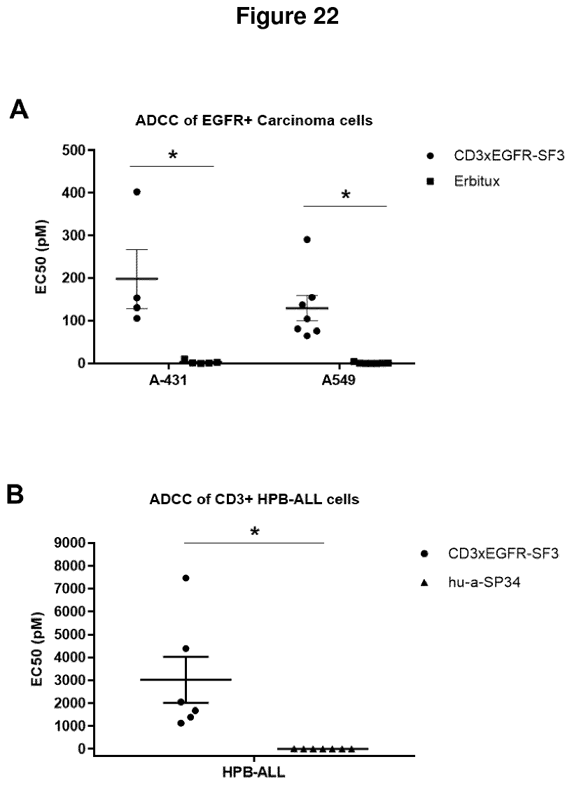

FIG. 22. CD3×EGFR-SF3 has a low antibody-dependent cell-mediated cytotoxicity potential. Antibody-dependent cell-mediated cytotoxicity (ADCC) of CD3×EGFR was evaluated in the EGFR+ carcinoma cell lines A-431 and A549 (A) as well as in CD3+ HPB-ALL cells (B) and represented by the EC₅₀ values that were extracted from the sigmoidal dose-response curves of specific killing. The error bars represent means±SEM from two independent experiments. Effect of the treatment was statistically significant for EGFR+ carcinoma cells (Least Square model, F=29, p<0.0001) and CD3+ HPB-ALL cells (T test, t=3, p<0.05). Statistically significant differences (p<0.05) are represented by asterisks (*).

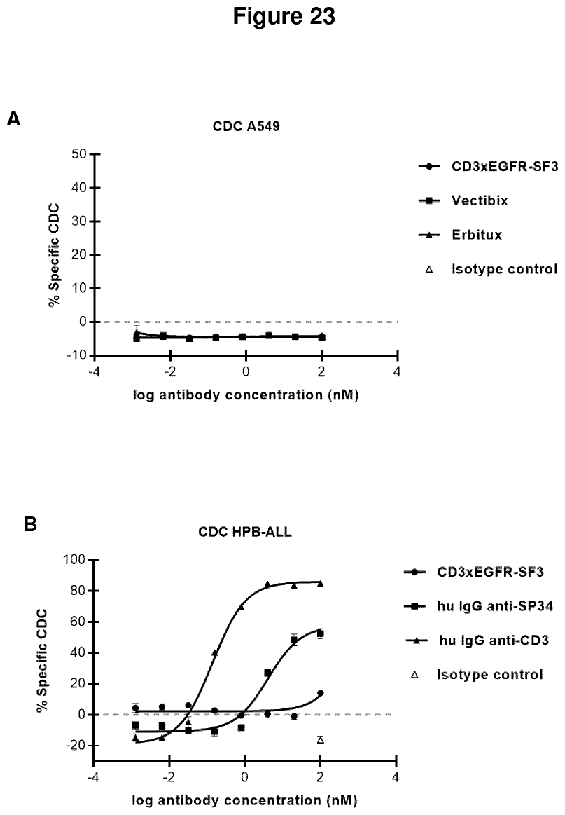

FIG. 23. CD3×EGFR-SF3 has no complement-dependent cytotoxicity. Specific complement-dependent cytotoxicity (CDC) was evaluated in the EGFR+ carcinoma cells A549 (A) as well as in CD3+ HPB-ALL cells (B) and the sigmoidal dose-response curves of specific CDC are represented.

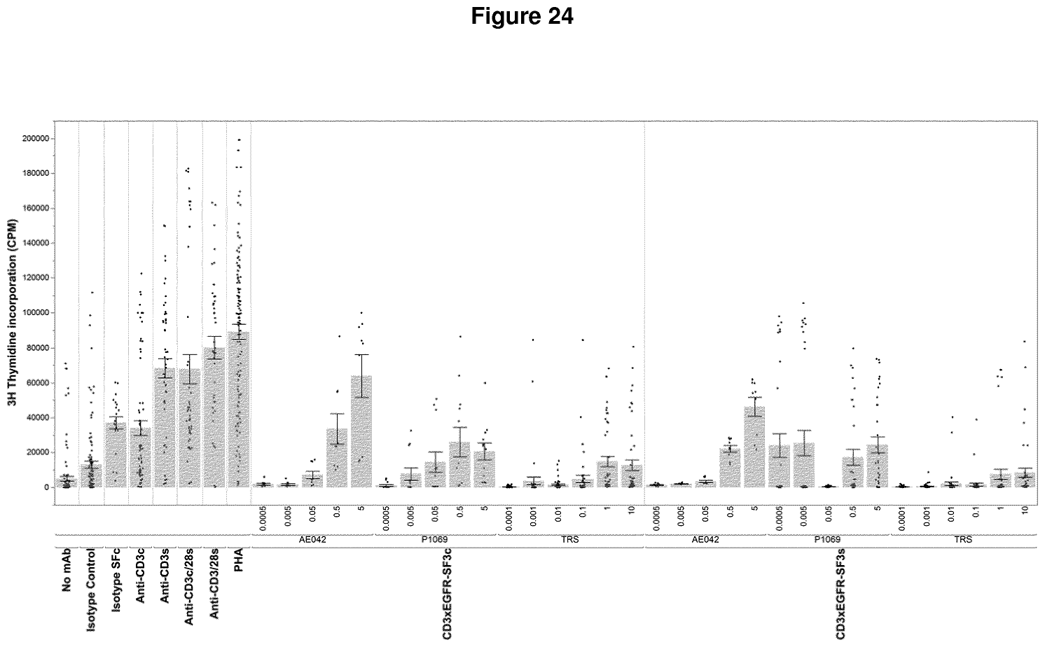

FIG. 24. Effects of CD3×EGFR-SF3 on the proliferation of PBMCs. PBMCs were incubated for 48 h in presence of increasing doses of CD3×EGFR or controls. The graph shows the results of 3H-thymidine incorporation from six independent experiments. AE042, P1069, and TRS represent different batches of CD3×EGFR-SF3 and 0.0005, 0.005, 0.05, 0.5 and 5 the concentrations in ug/ml. For the different treatments, “c” stands for coated and “s” for soluble. The error bars represent means±SEM.

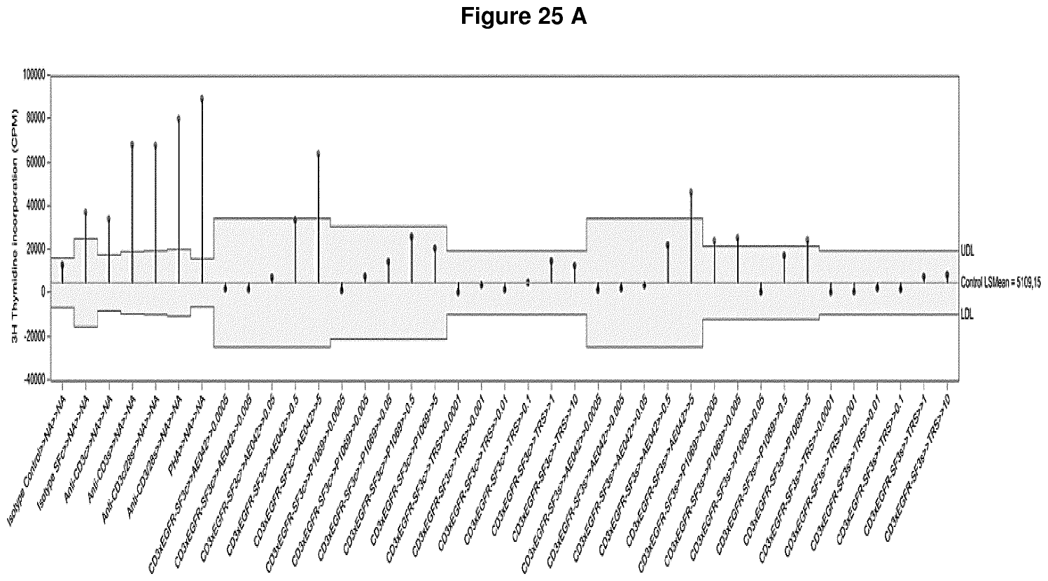

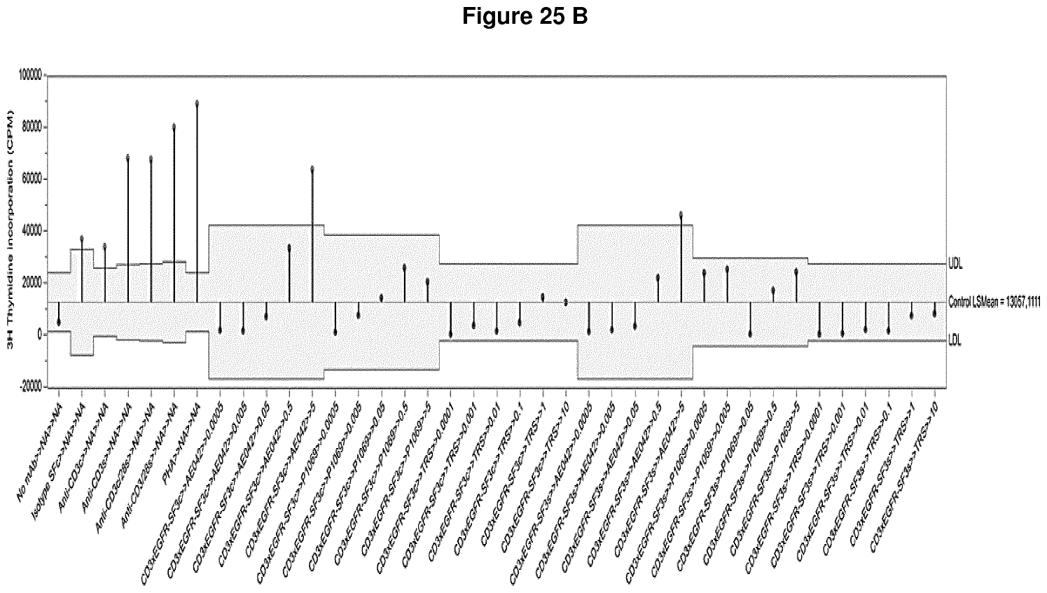

FIG. 25. Statistical analysis of the effects of CD3×EGFR-SF3 on the proliferation of PBMCs. Data from FIG. 24 were analyzed by fit least square model followed by a Dunnett's comparison (α=0.05) to compare the means against the no mAb control (A) and against the isotype control (B). Significant differences of the mean are shown as the needles bars outside of the decision limits (95% CI interval for each treatment; gray area).

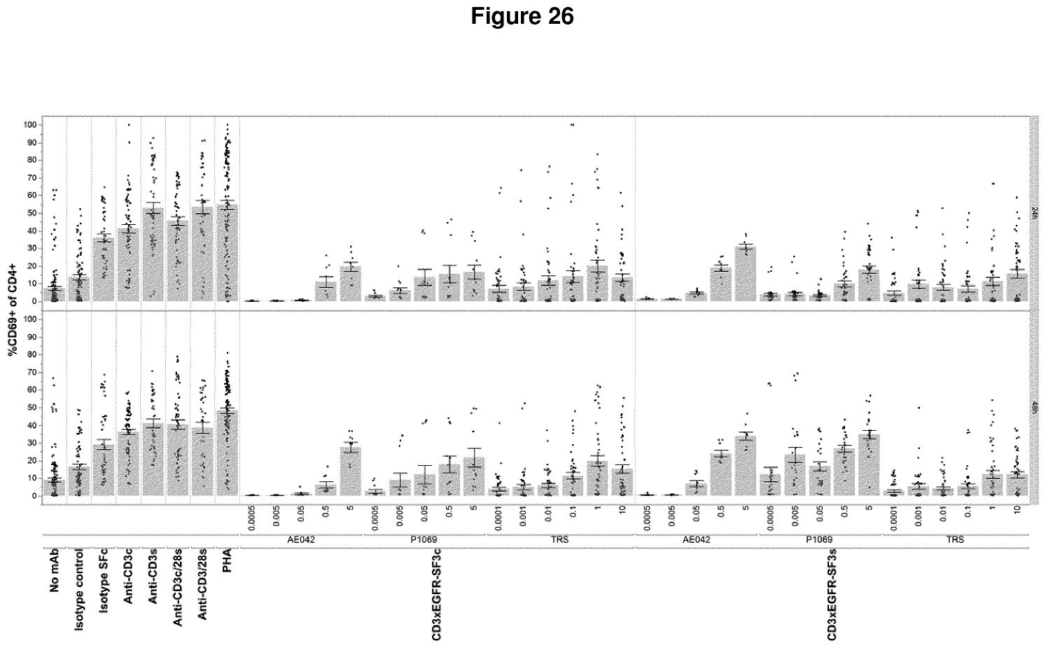

FIG. 26. Non-specific CD4+ T cell activation in response to CD3×EGFR-SF3. PBMCs were incubated for 24 h or 48 h in presence of increasing doses of CD3×EGFR or controls. Activation of CD4+ T cell was measured as the expression of the activation marker CD69 by flow cytometry. AE042, P1069, and TRS represent different batches of CD3×EGFR-SF3 and 0.0005, 0.005, 0.05, 0.5 and 5 the concentrations in ug/ml. For the different treatments, “c” stands for coated and “s” for soluble. The error bars represent means±SEM from six independent experiments.

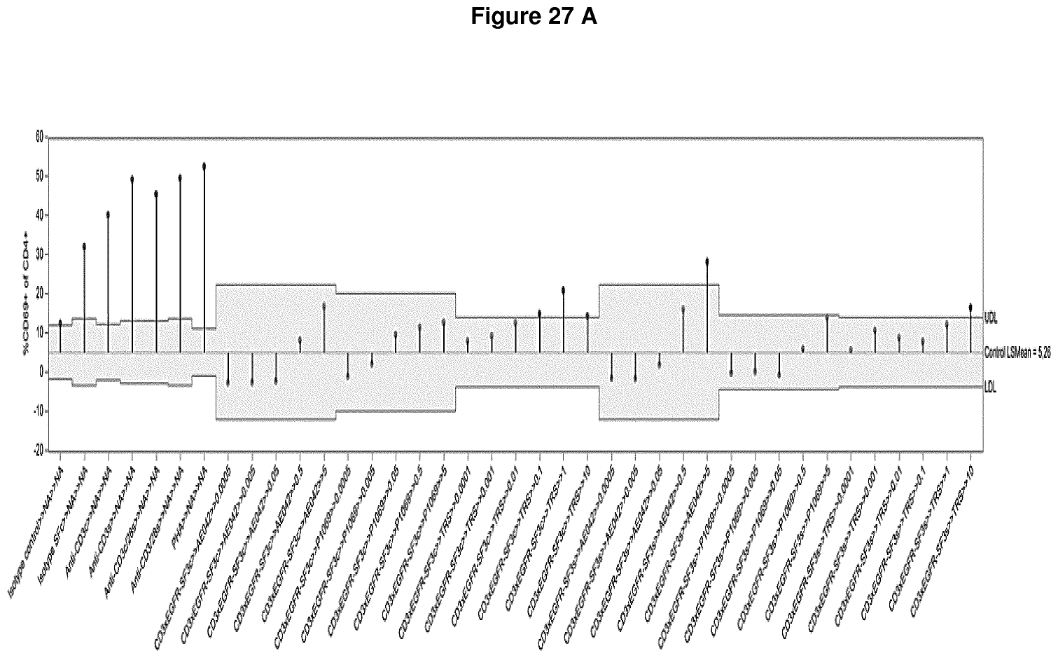

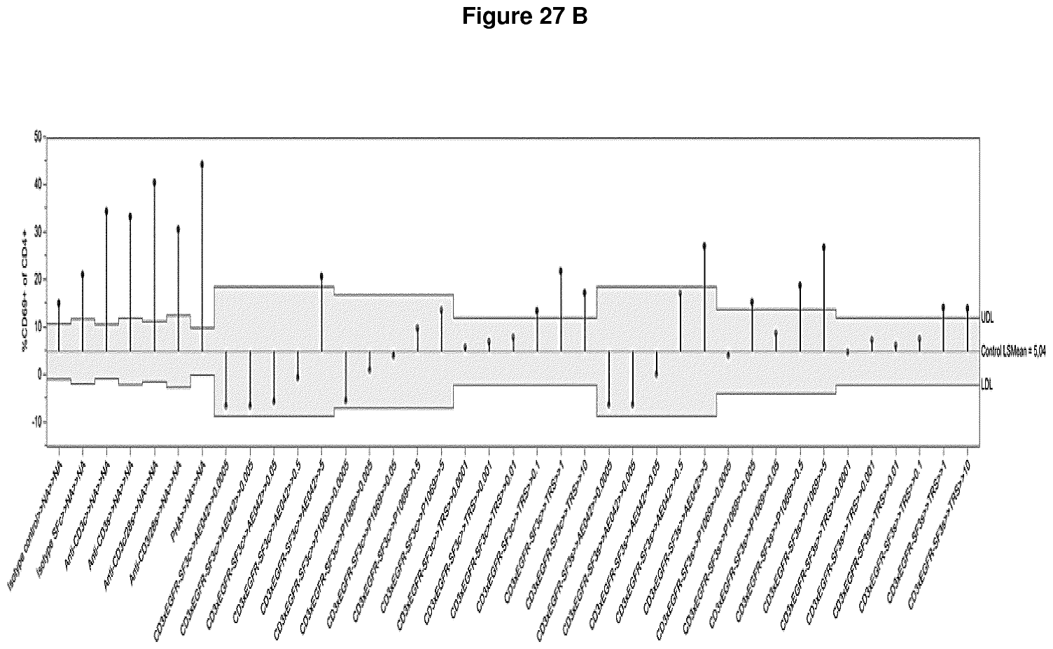

FIG. 27. Statistical comparison of the non-specific CD4+ T cell activation between CD3×EGFR-SF3 and no mAb condition. Data from FIG. 26 were analyzed by fit least square model followed by a Dunnett's comparison (α=0.05) to compare the means against the no mAb control at 24 h (A) and 48 h (B). Significant differences of the mean are shown as the needles bars outside of the decision limits (95% CI interval for each treatment; gray area).

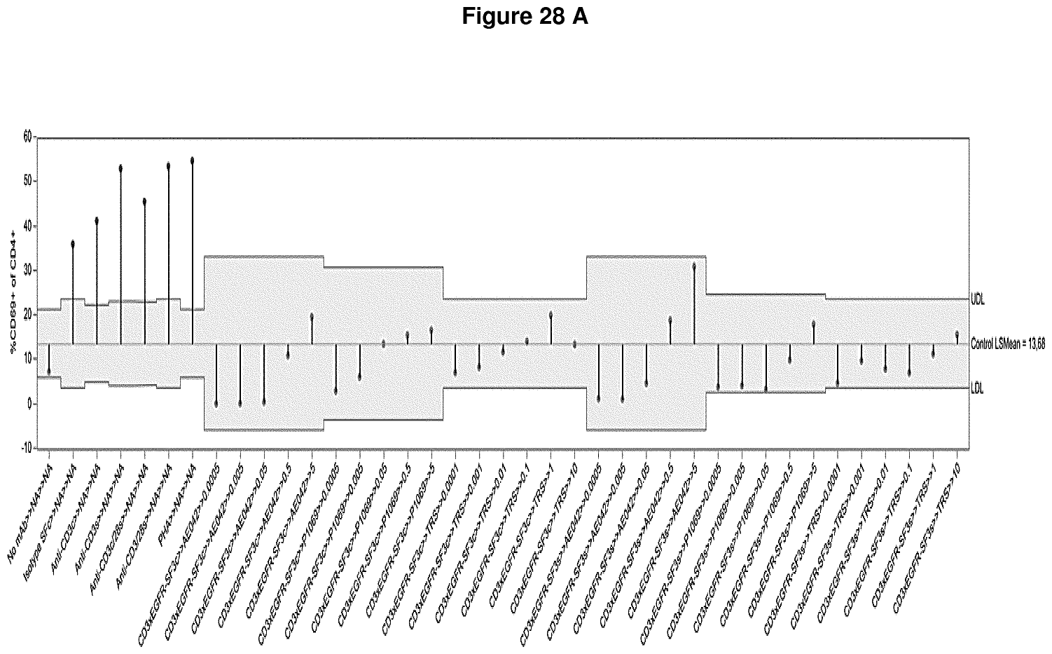

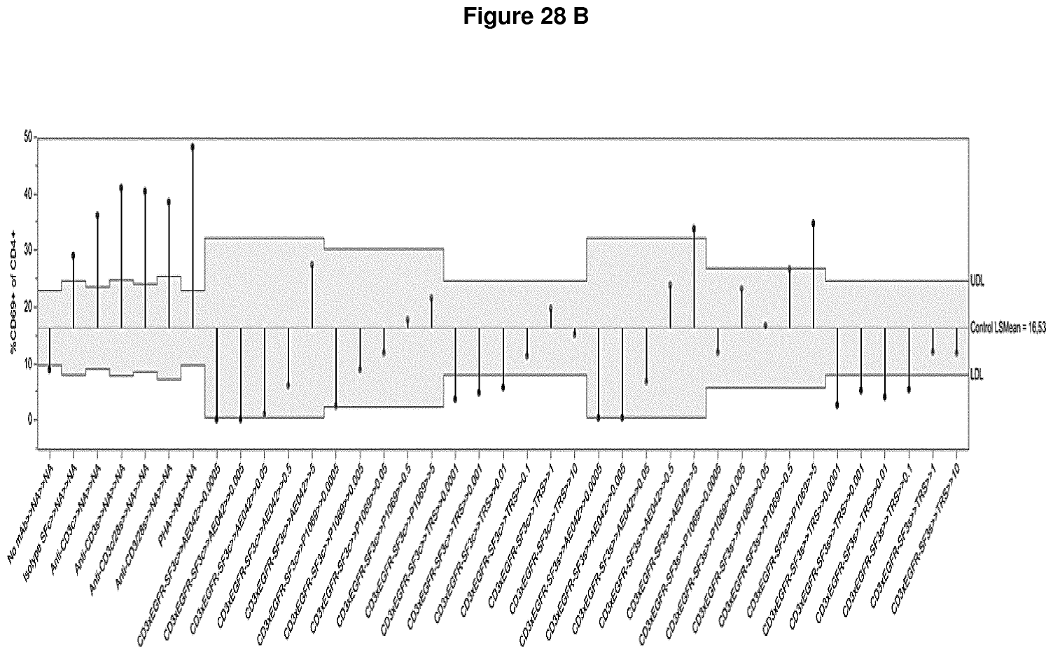

FIG. 28. Statistical comparison of the non-specific CD4+ T cell activation between CD3×EGFR-SF3 and the isotype control. Data from FIG. 26 were analyzed by fit least square model followed by a Dunnett's comparison (α=0.05) to compare the means against the isotype control at 24 h (A) and 48 h (B). Significant differences of the mean are shown as the needles bars outside of the decision limits (95% CI interval for each treatment; gray area).

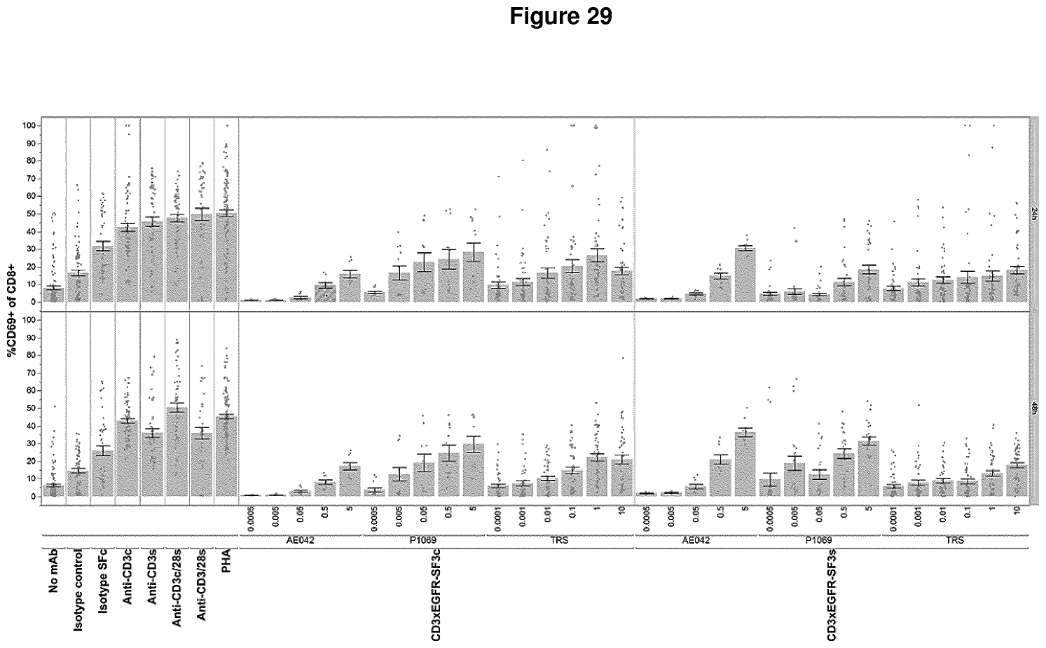

FIG. 29. Non-specific CD8+ T cell activation in response to CD3×EGFR-SF3. PBMCs were incubated for 24 h or 48 h in presence of increasing doses of CD3×EGFR-SF3 or controls. Activation of CD8+ T cell was measured as the expression of the activation marker CD69 by flow cytometry. AE042, P1069, and TRS represent different batches of CD3×EGFR-SF3 and 0.0005, 0.005, 0.05, 0.5 and 5 the concentrations in ug/ml. For the different treatments, “c” stands for coated and “s” for soluble. The error bars represent means±SEM from six independent experiments.

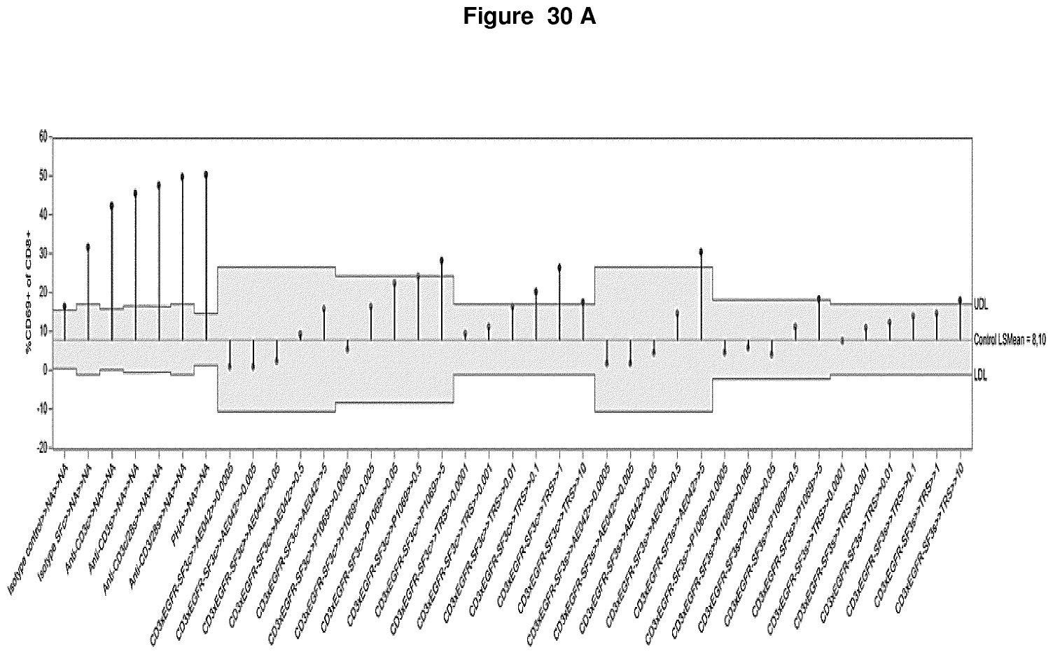

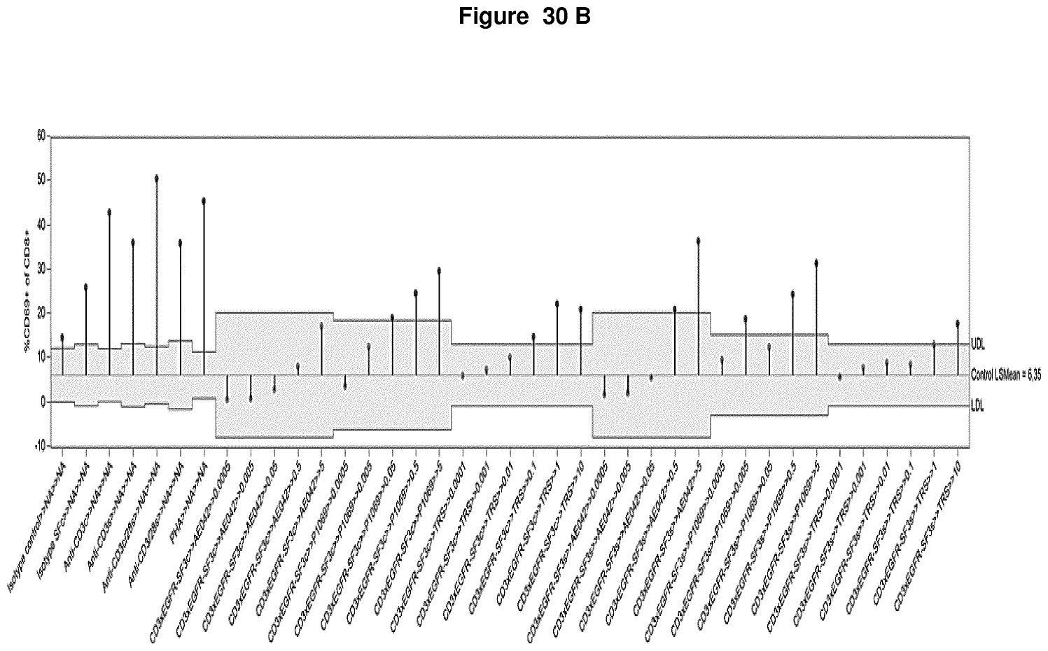

FIG. 30. Statistical comparison of the non-specific CD8+ T cell activation between CD3×EGFR-SF3 and no mAb condition. Data from FIG. 29 were analyzed by fit least square model followed by a Dunnett's comparison (α=0.05) to compare the means against the no mAb control at 24 h (A) and 48 h (B). Significant differences of the mean are shown as the needles bars outside of the decision limits (95% CI interval for each treatment; gray area).

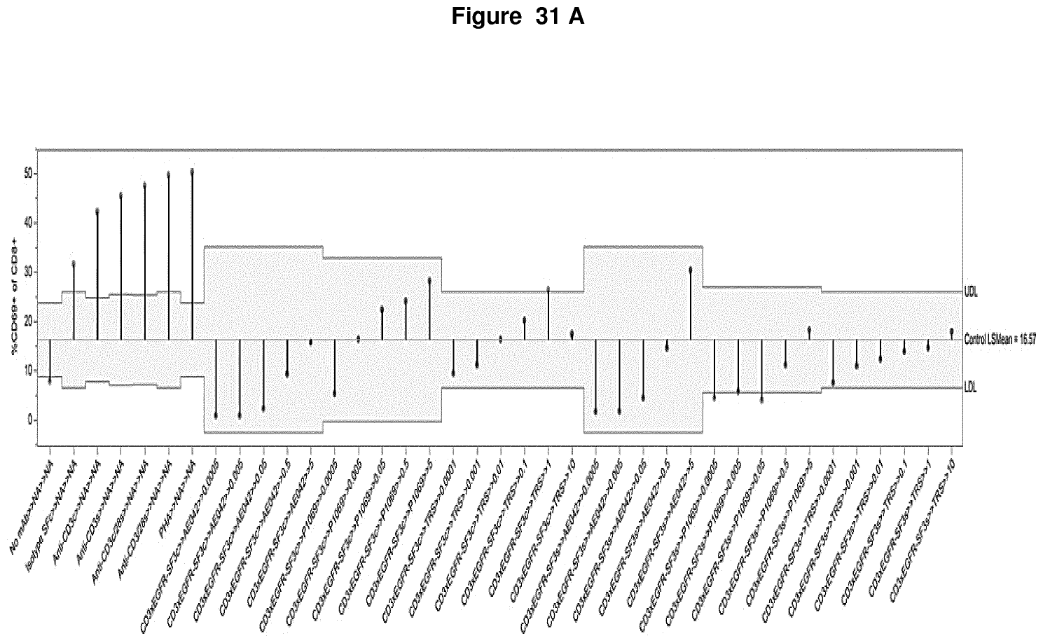

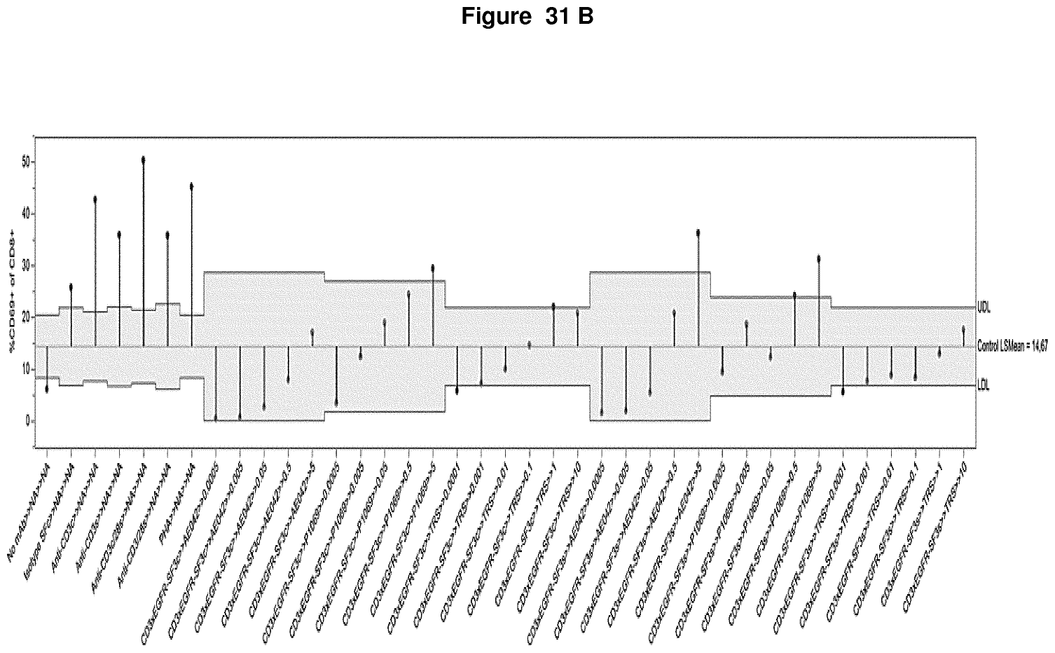

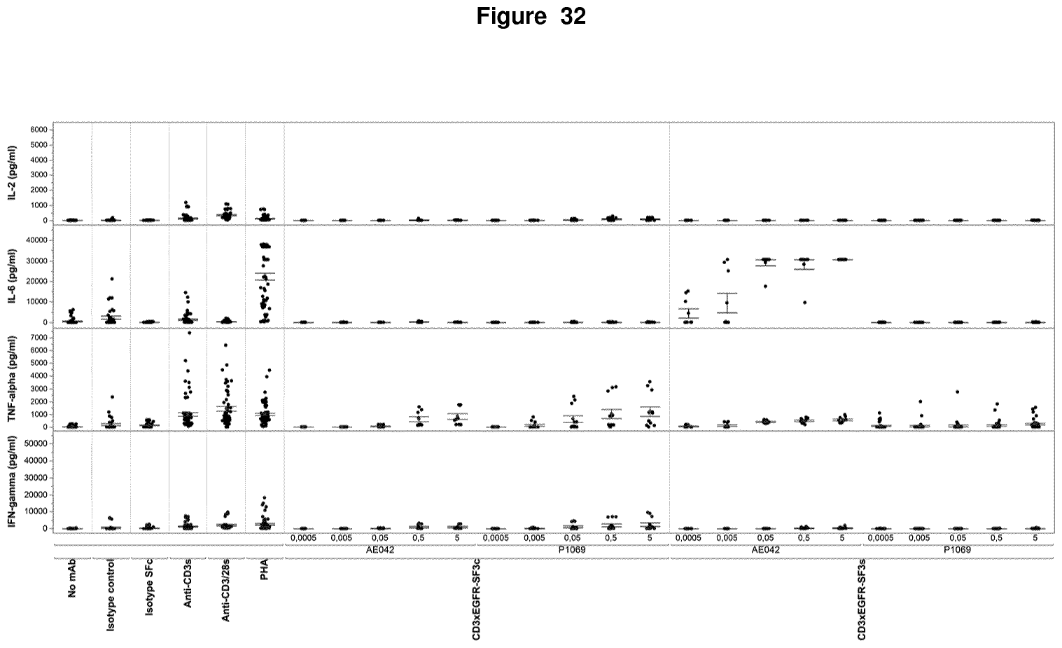

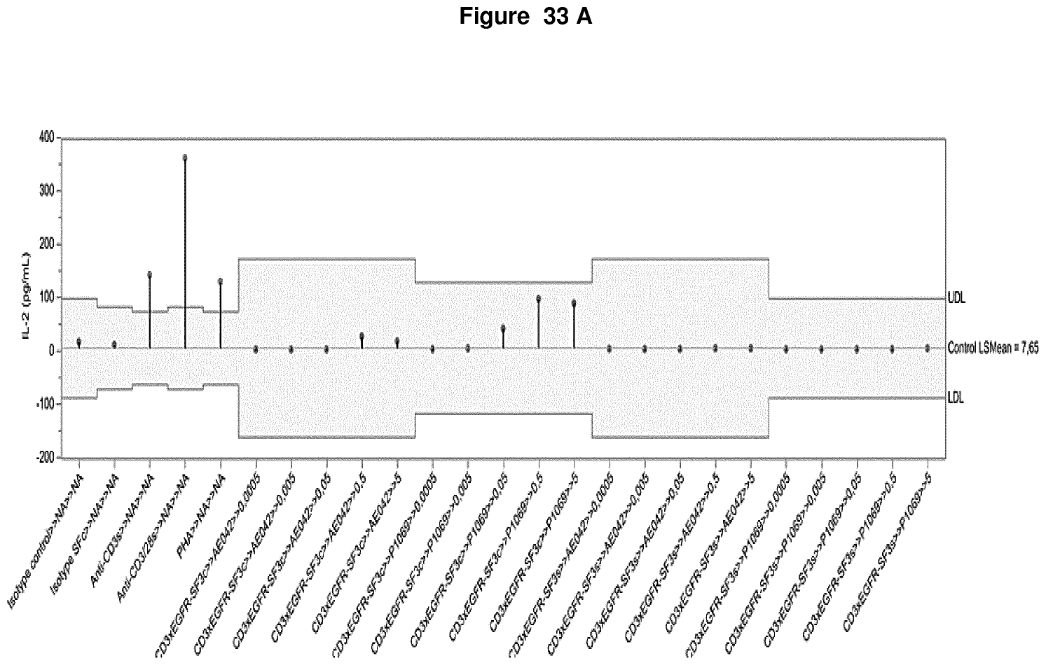

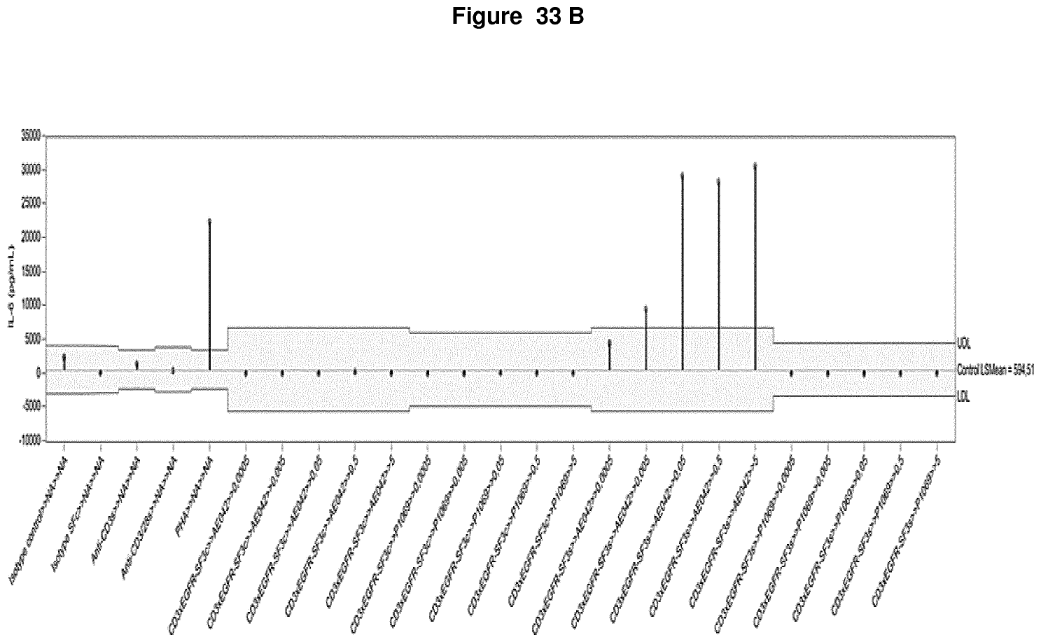

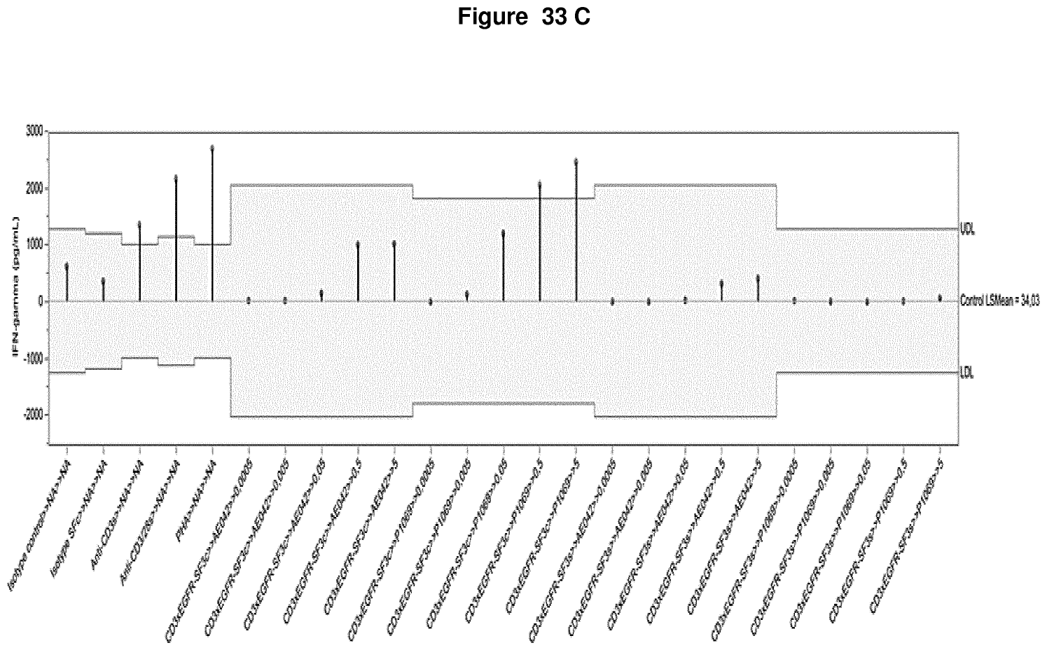

FIG. 31. Statistical comparison of the non-specific CD8+ T cell activation between CD3×EGFR-SF3 and the isotype control. Data from FIG. 29 were analyzed by fit least square model followed by a Dunnett's comparison (α=0.05) to compare the means against the isotype control at 24 h (A) and 48 h (B). Significant differences of the mean are shown as the needles bars outside of the decision limits (95% CI interval for each treatment; gray area).

FIG. 32. Non-specific T cell cytokine responses to CD3×EGFR-SF3 at 24 h. PBMCs were incubated for 24 h in presence of increasing doses of CD3×EGFR-SF3 or controls, and the levels of IL-2, IL-6, TNF-α, and IFN-γ released were measured by Luminex in the supernatant. AE042 and P1069, represent different batches of CD3×EGFR-SF3 and 0.0005, 0.005, 0.05, 0.5 and 5 the concentrations in ug/ml. For the different treatments, “c” stands for coated and “s” for soluble. The error bars represent means±SEM from six independent experiments.

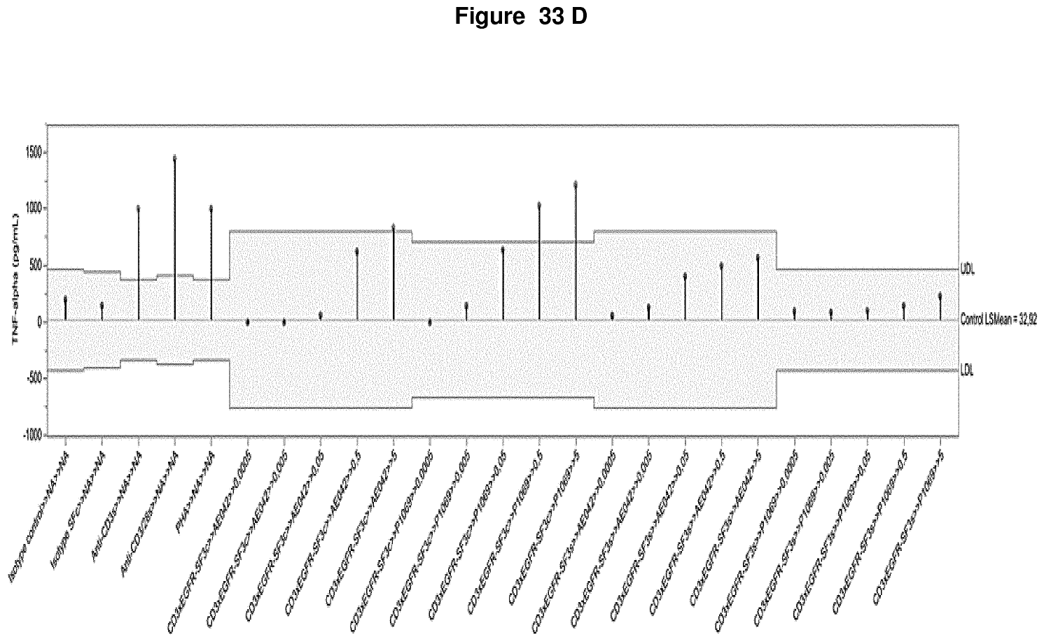

FIG. 33. Statistical comparison of the non-specific T cell cytokine responses between CD3×EGFR-SF3 and the no mAb condition at 24 h. Data from FIG. 32 were analyzed by fit least square model followed by a Dunnett's comparison (α=0.05) to compare the means against the no mAb control of IL-2 (A), IL-6 (B), IFN-γ (C), and TNF-α (D). Significant differences of the mean are shown as the needles bars outside of the decision limits (95% CI interval for each treatment; gray area).

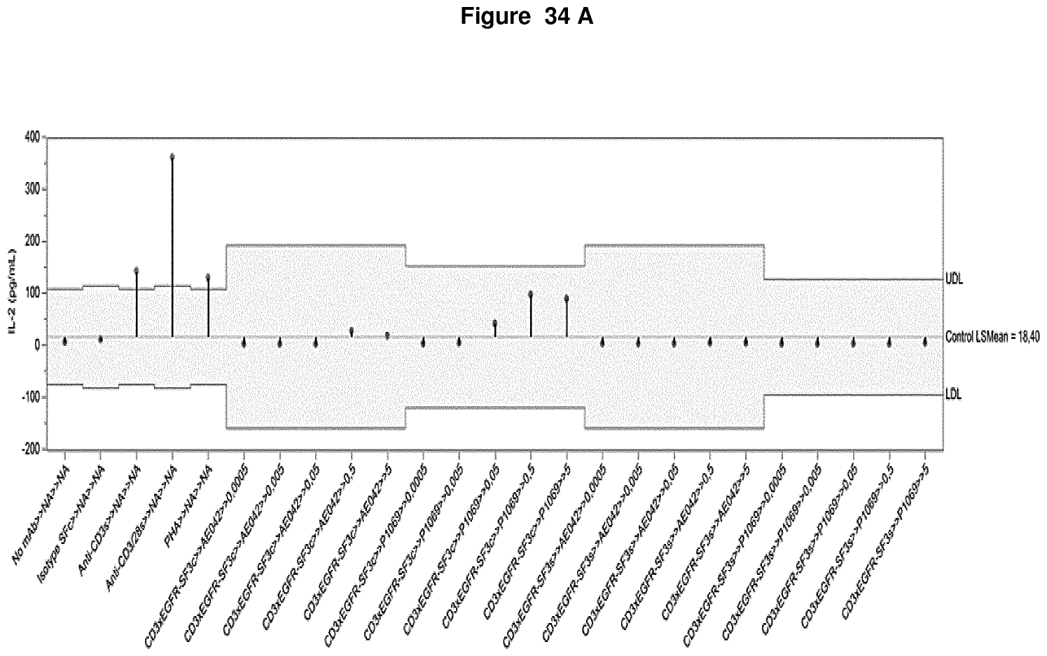

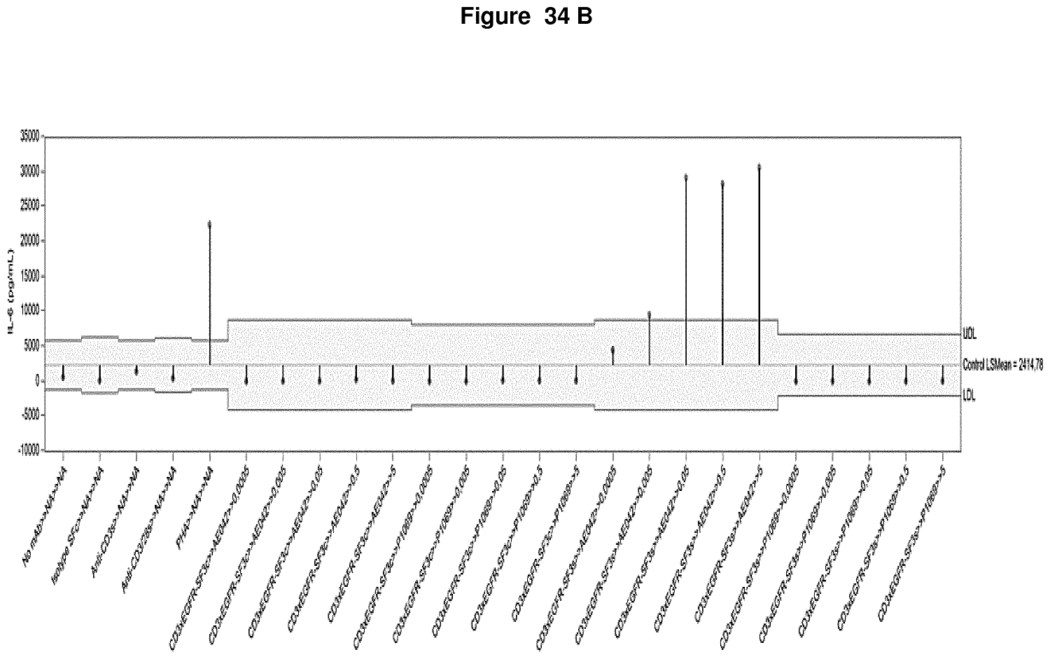

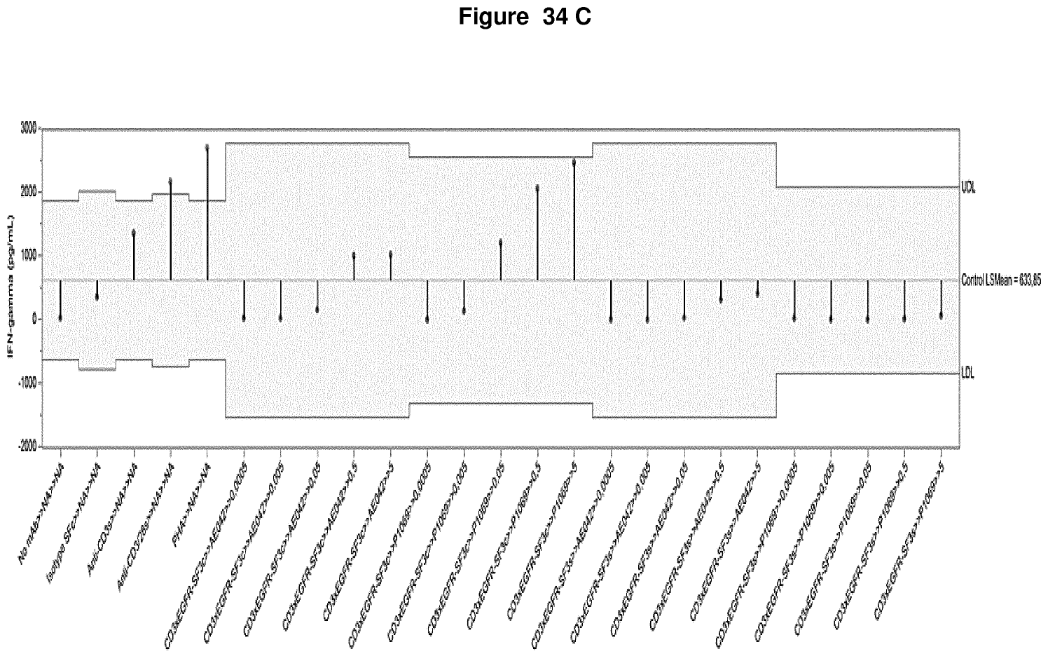

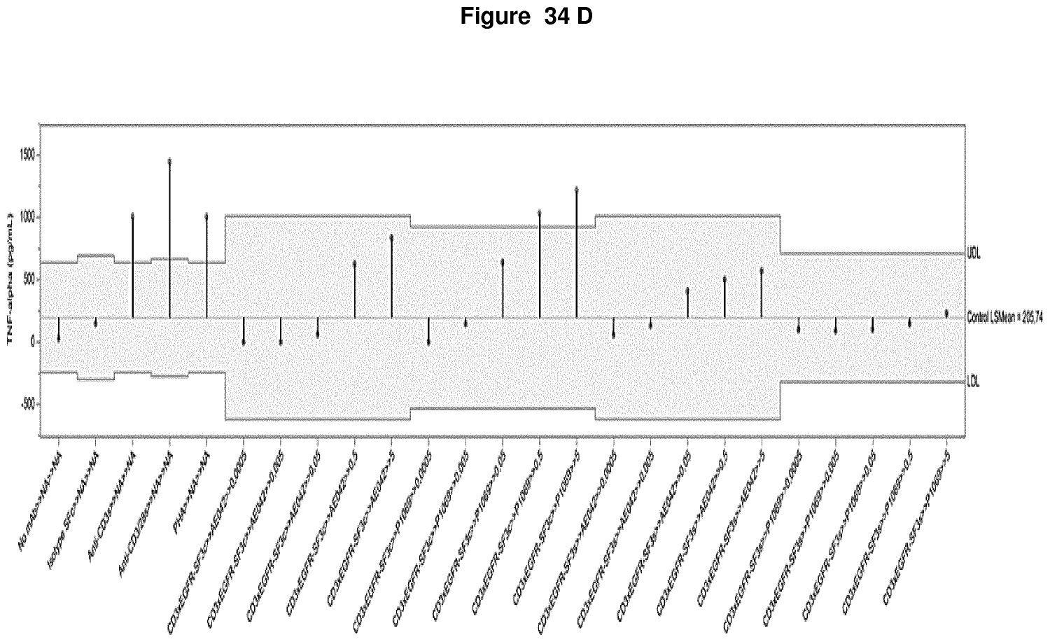

FIG. 34. Statistical comparison of the non-specific T cell cytokine responses between CD3×EGFR-SF3 and the isotype control at 24 h. Data from FIG. 32 were analyzed by fit least square model followed by a Dunnett's comparison (α=0.05) to compare the means against the isotype control of IL-2 (A), IL-6 (B), IFN-γ (C), and TNF-α (D). Significant differences of the mean are shown as the needles bars outside of the decision limits (95% CI interval for each treatment; gray area).

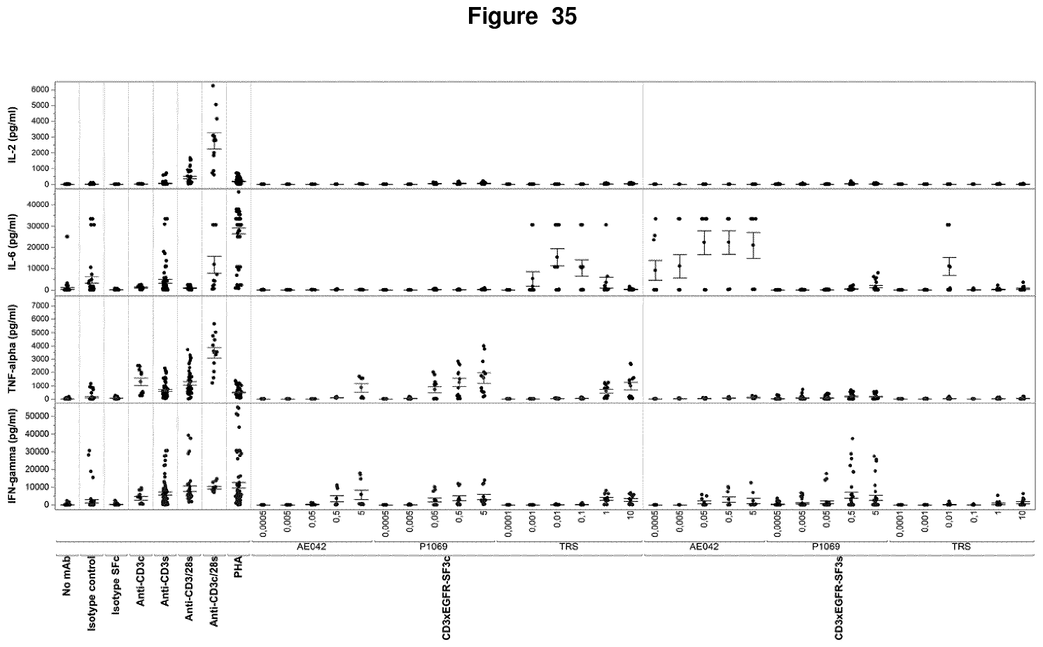

FIG. 35. Non-specific T cell cytokine responses to CD3×EGFR-SF3 at 48 h. PBMCs were incubated for 48 h in presence of increasing doses of CD3×EGFR-SF3 or controls, and the levels of IL-2, IL-6, TNF-α, and IFN-γ released were measured by Luminex in the supernatant. AE042, P1069, and TRS represent different batches of CD3×EGFR-SF3 and 0.0001, 0.0005, 0.001, 0.005, 0.01, 0.05, 0.1, 0.5, 1, 5, and 10 the concentrations in ug/ml. For the different treatments, “c” stands for coated and “s” for soluble. The error bars represent means±SEM from six independent experiment.

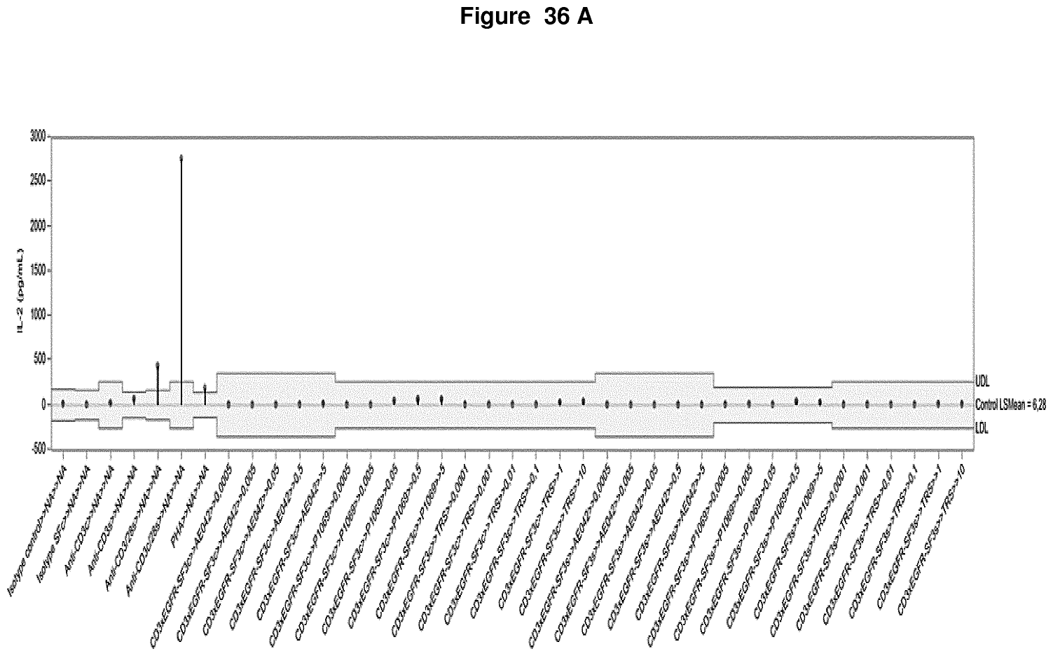

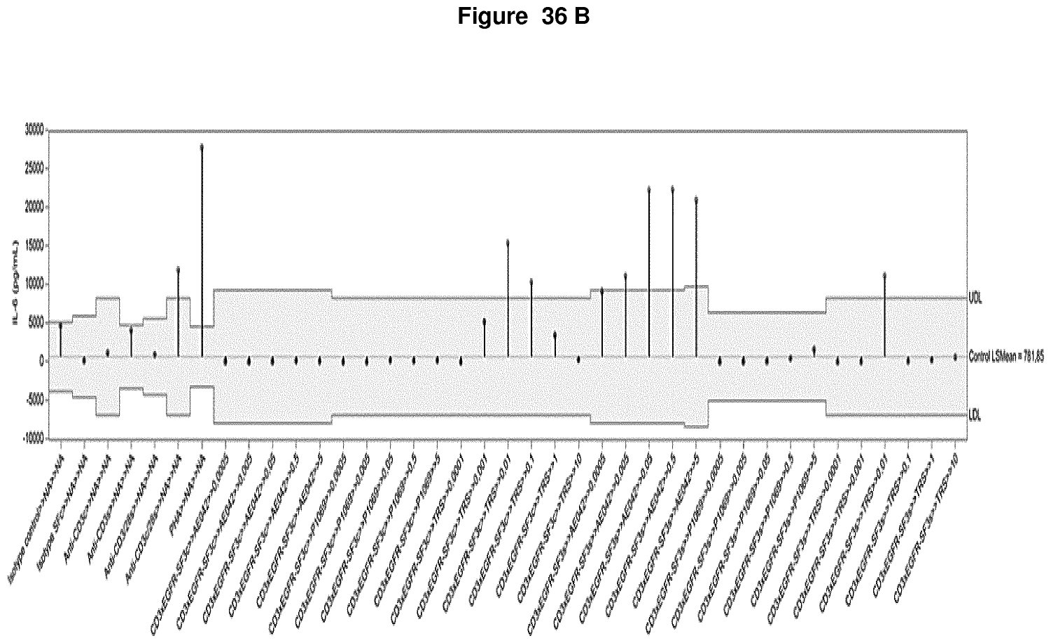

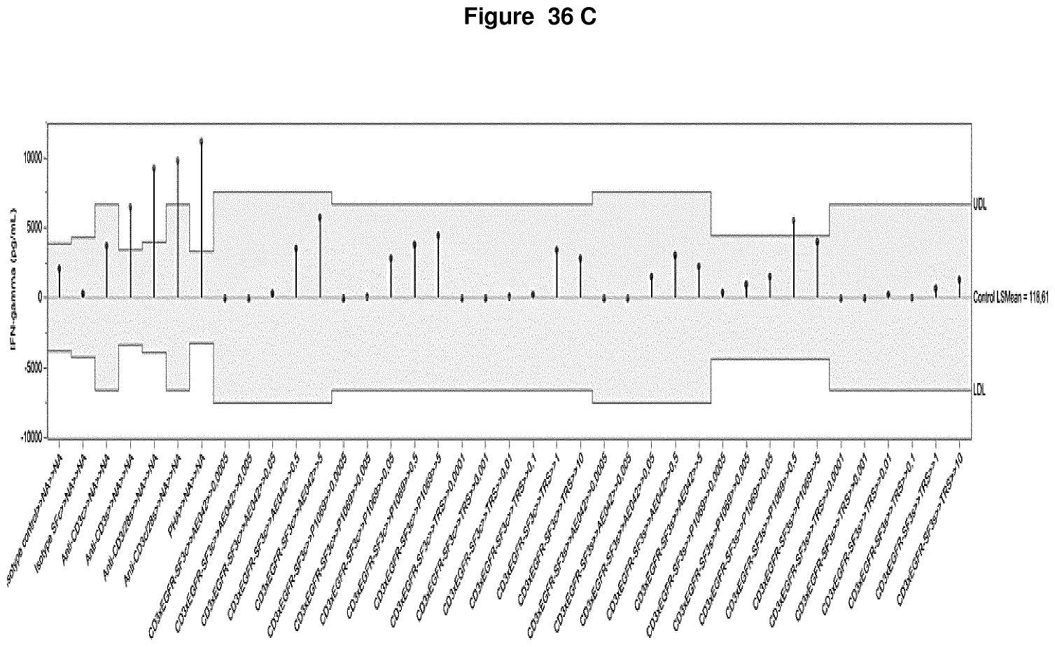

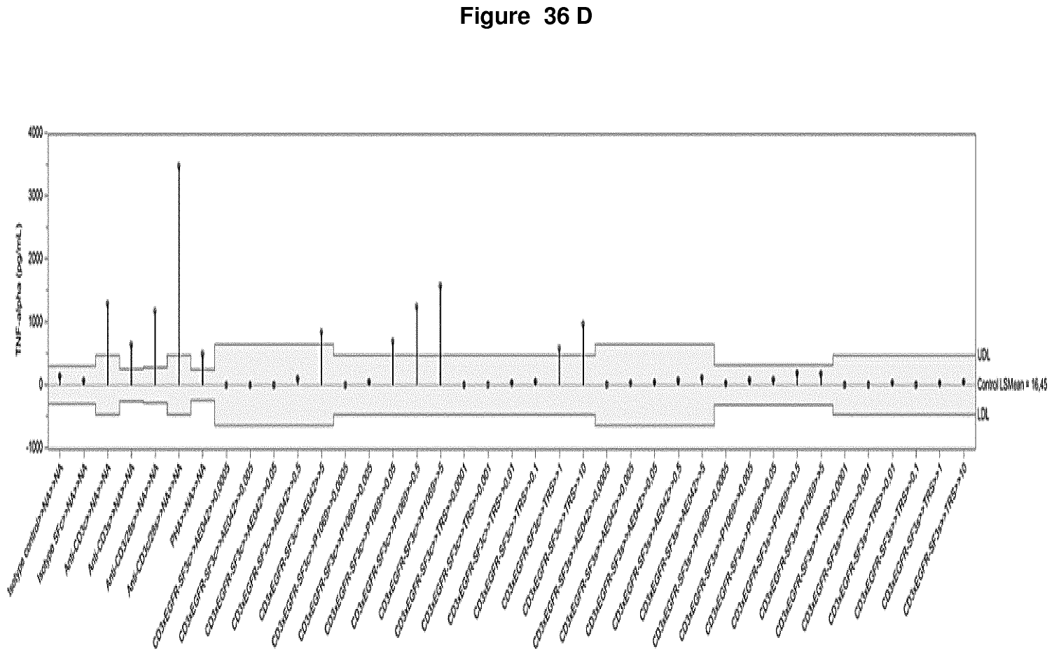

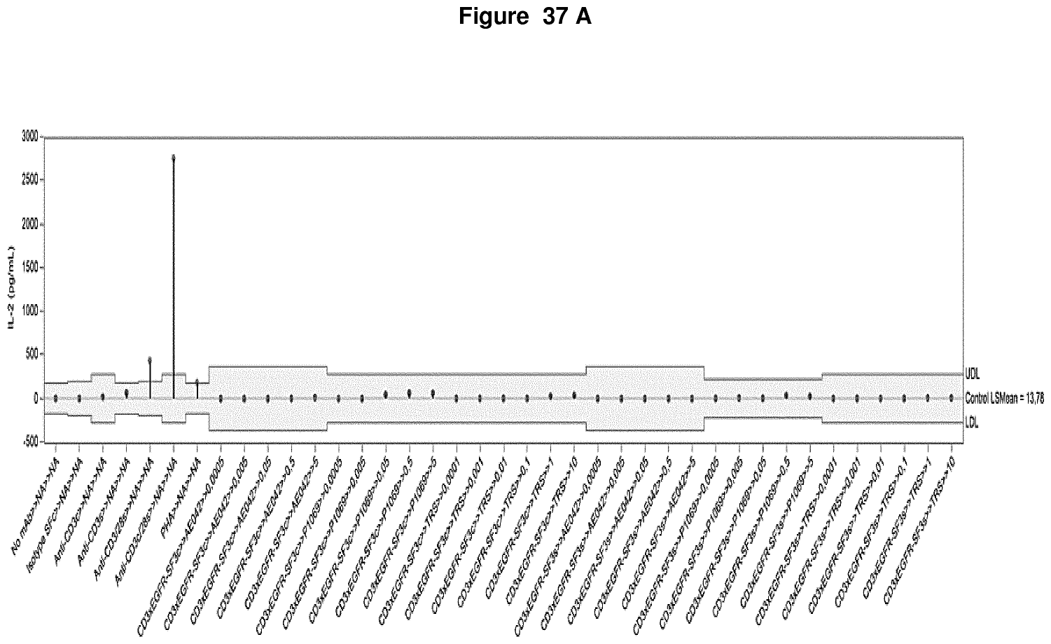

FIG. 36. Statistical comparison of the non-specific T cell cytokine responses between CD3×EGFR-SF3 and the no mAb condition at 48 h. Data from FIG. 35 were analyzed by fit least square model followed by a Dunnett's comparison (α=0.05) to compare the means against the no mAb control of IL-2 (A), IL-6 (B), IFN-γ (C), and TNF-α (D). Significant differences of the mean are shown as the needles bars outside of the decision limits (95% CI interval for each treatment; gray area).

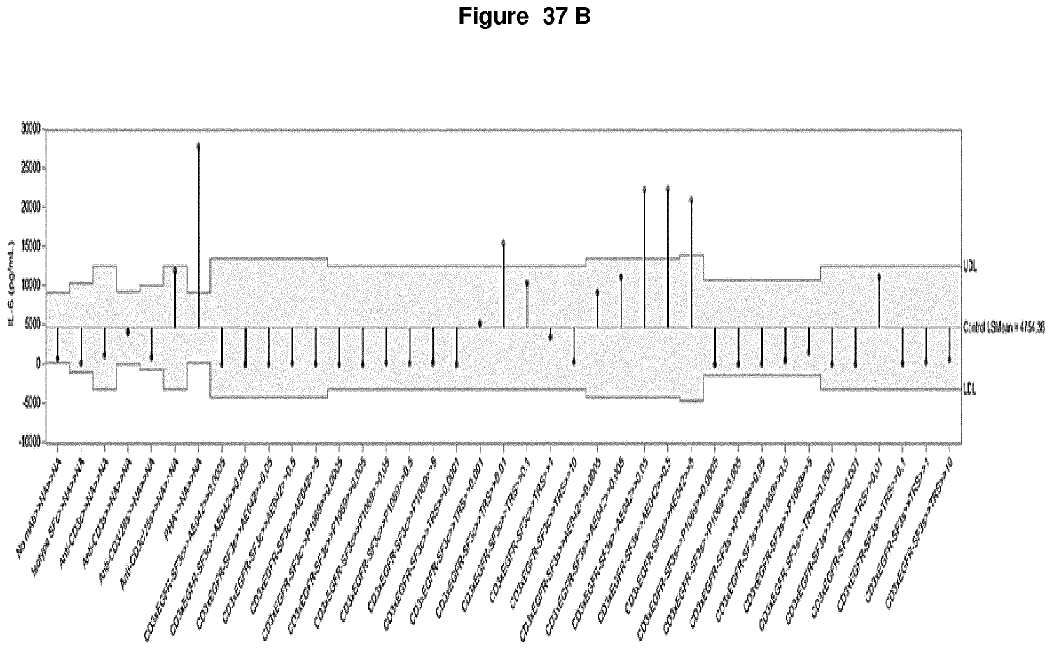

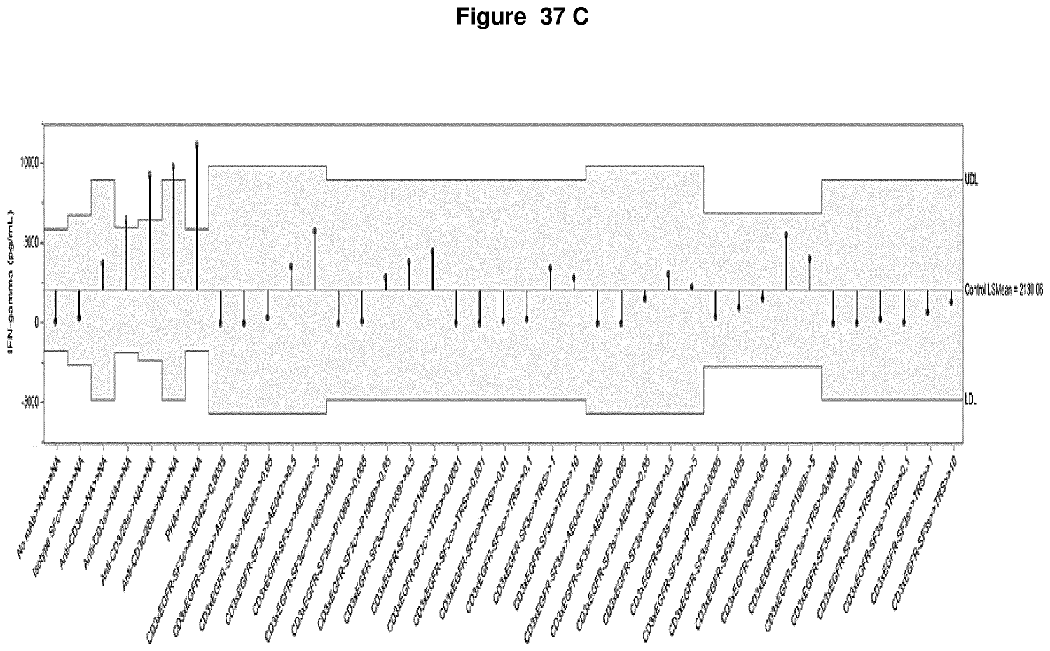

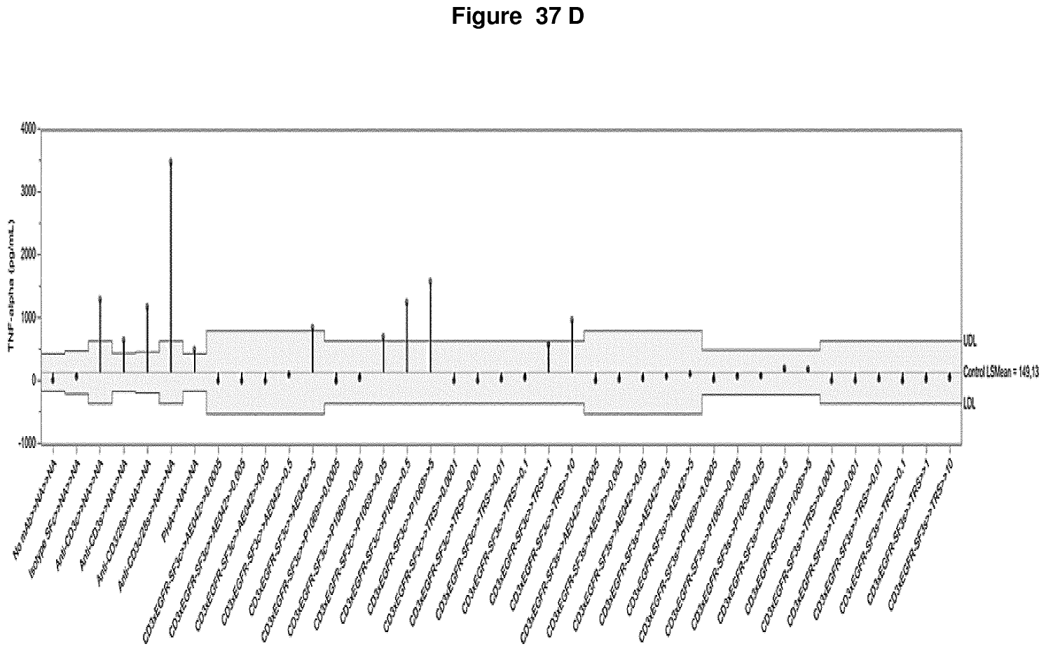

FIG. 37. Statistical comparison of the non-specific T cell cytokine responses between CD3×EGFR-SF3 and the isotype control at 48 h. Data from FIG. 35 were analyzed by fit least square model followed by a Dunnett's comparison (α=0.05) to compare the means against the isotype control of IL-2 (A), IL-6 (B), IFN-γ (C), and TNF-α (D). Significant differences of the mean are shown as the needles bars outside of the decision limits (95% CI interval for each treatment; gray area).

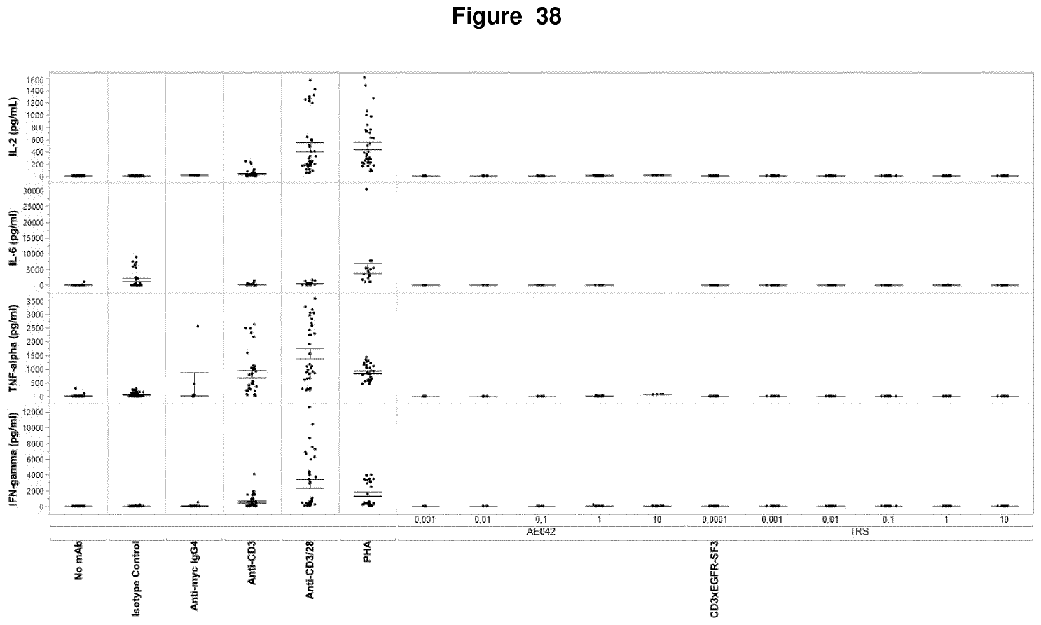

FIG. 38. CD3×EGFR-SF3 does not induce a non-specific T cell cytokine response in a high density PBMC assay. PBMCs were incubated for 48 h at high density (10⁷ cells/ml). The cells were then plated at a normal density (10⁶ cells/ml), and cultured for 24 h in presence of increasing doses of CD3×EGFR-SF3 or controls, and the levels of IL-2, IL-6, TNF-α, and IFN-γ released were measured by Luminex in the supernatant. AE042 and TRS represent different batches of CD3×EGFR-SF3 and 0.0001, 0.001, 0.01, 0.1, 1 and 10 the concentrations in ug/ml. The error bars represent means±SEM from four independent experiment.

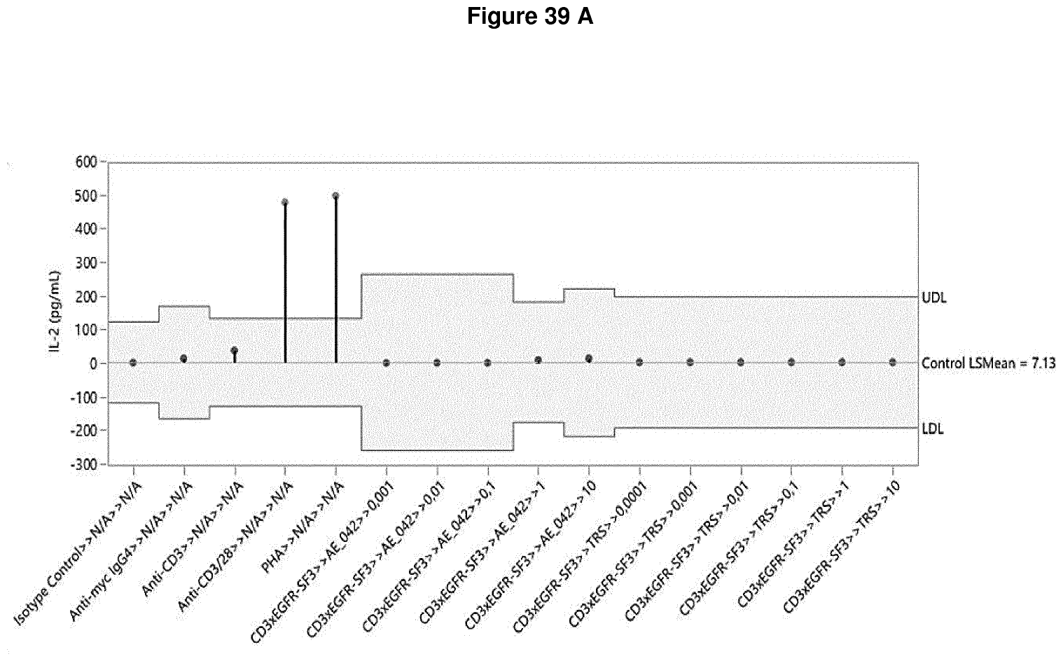

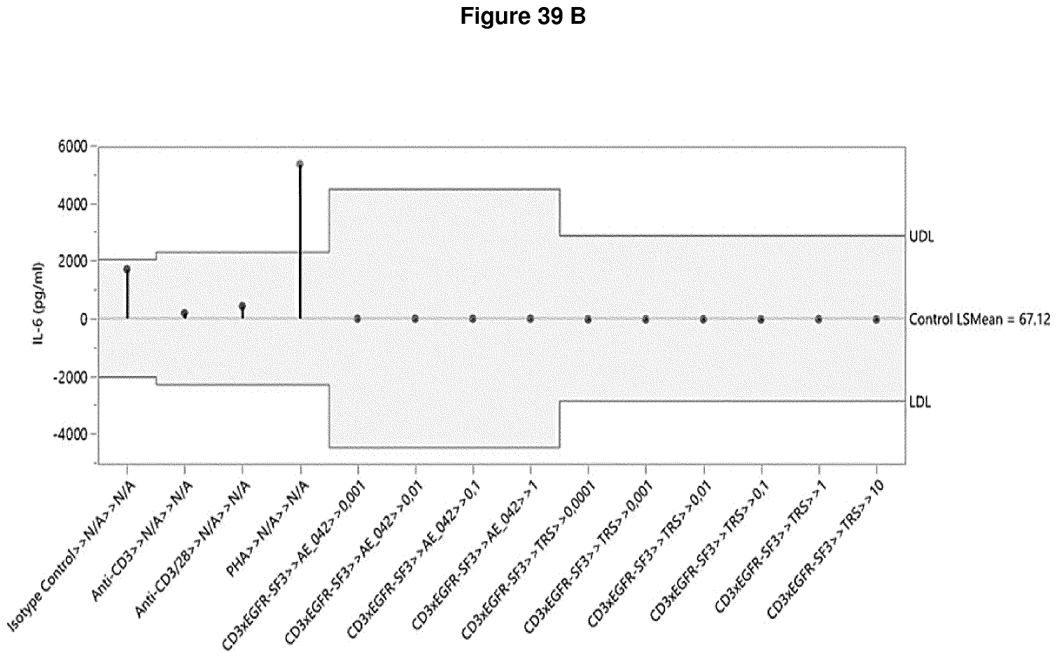

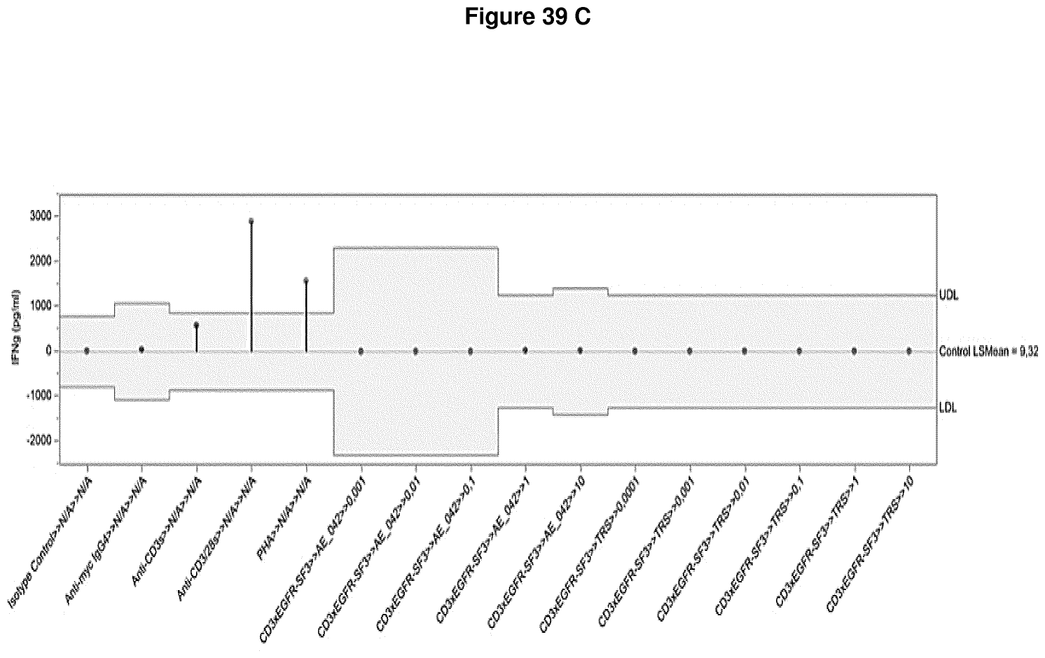

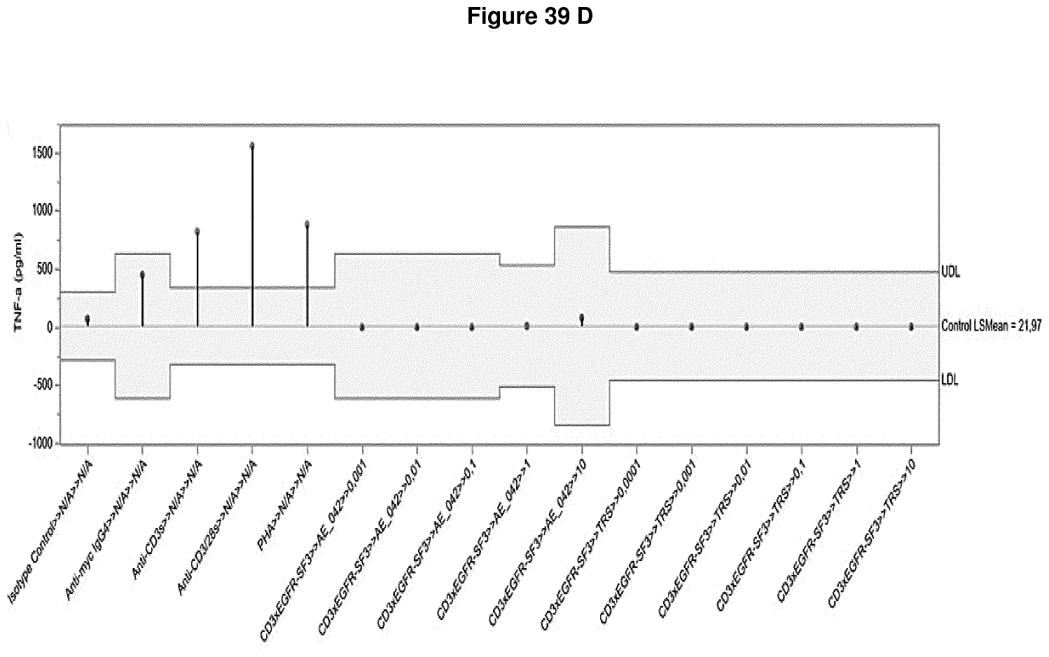

FIG. 39. Statistical comparison of the non-specific T cell cytokine responses between CD3×EGFR-SF3 and the no mAb condition in a high density PBMC assay. Data from FIG. 38 were analyzed by fit least square model followed by a Dunnett's comparison (α=0.05) to compare the means against the no mAb control of IL-2 (A), IL-6 (B), IFN-γ (C), and TNF-α (D). Significant differences of the mean are shown as the needles bars outside of the decision limits (95% CI interval for each treatment; gray area).

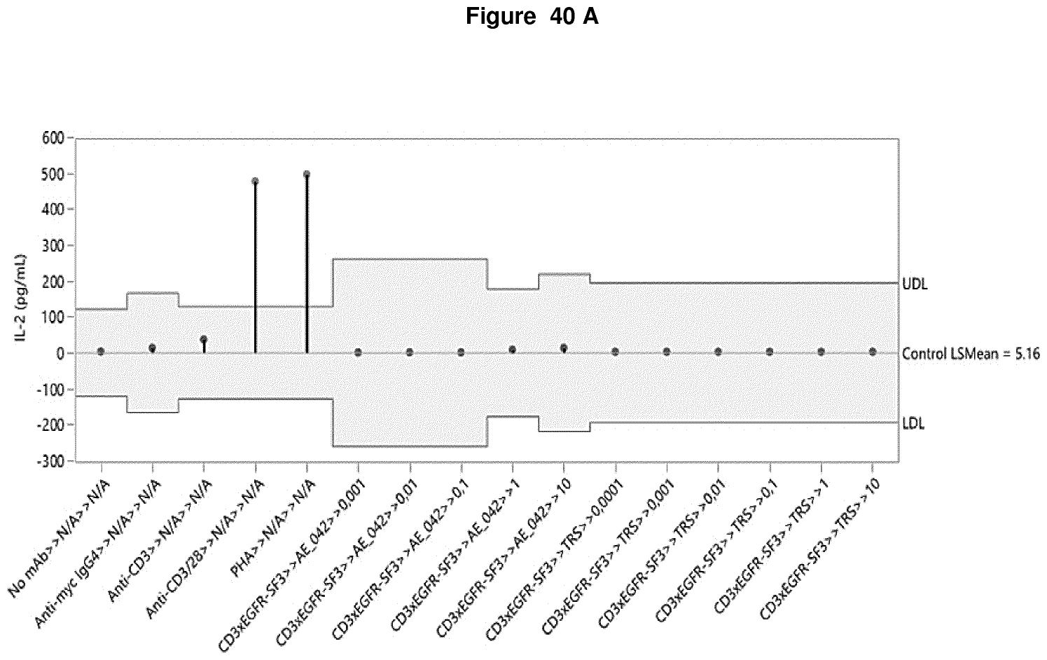

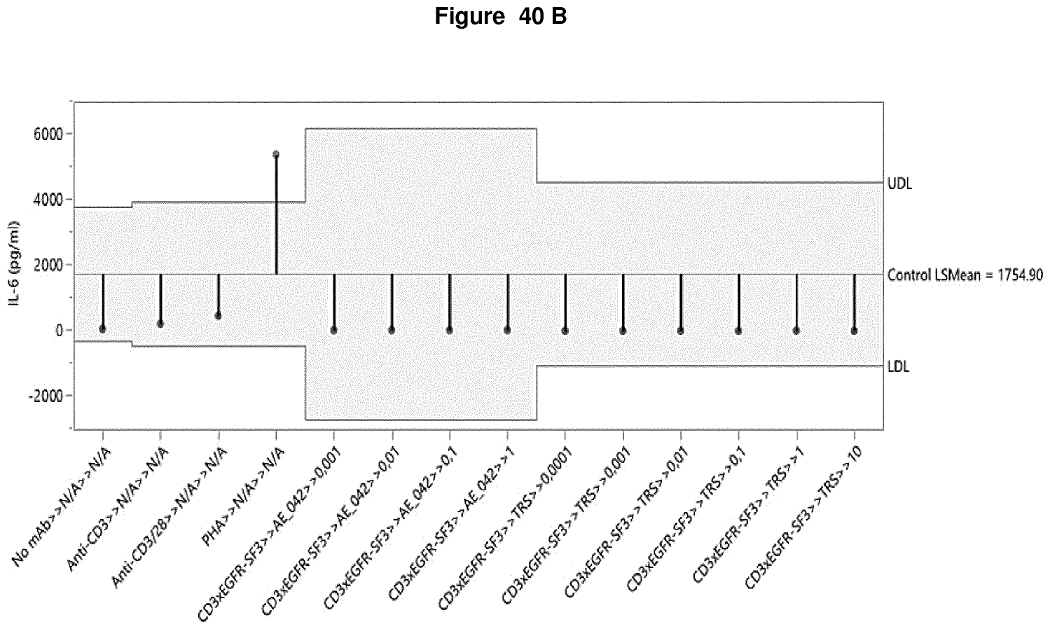

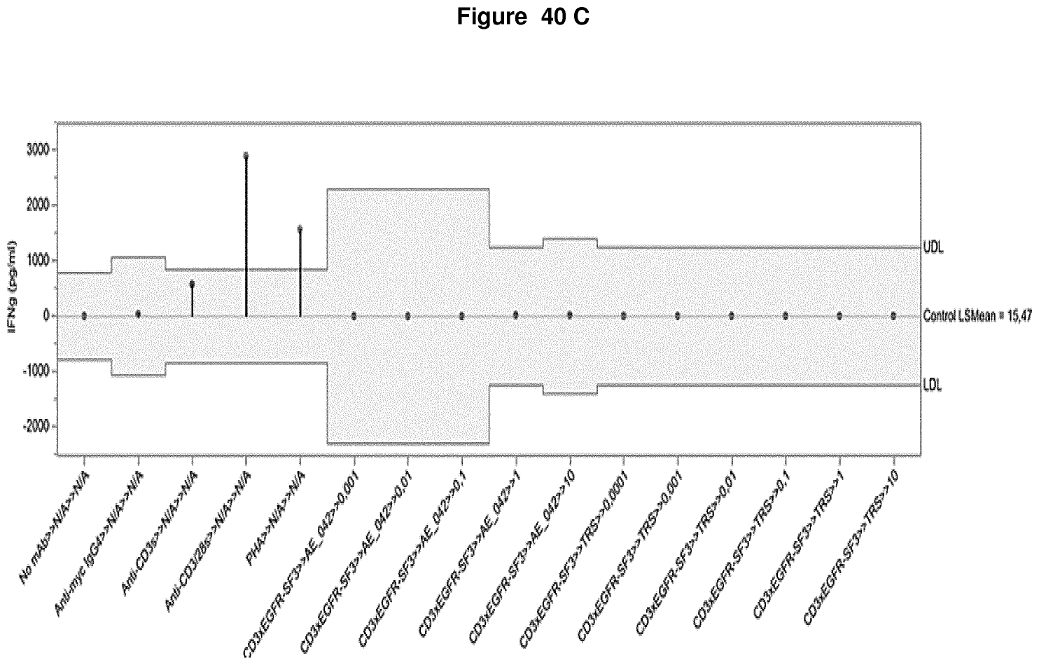

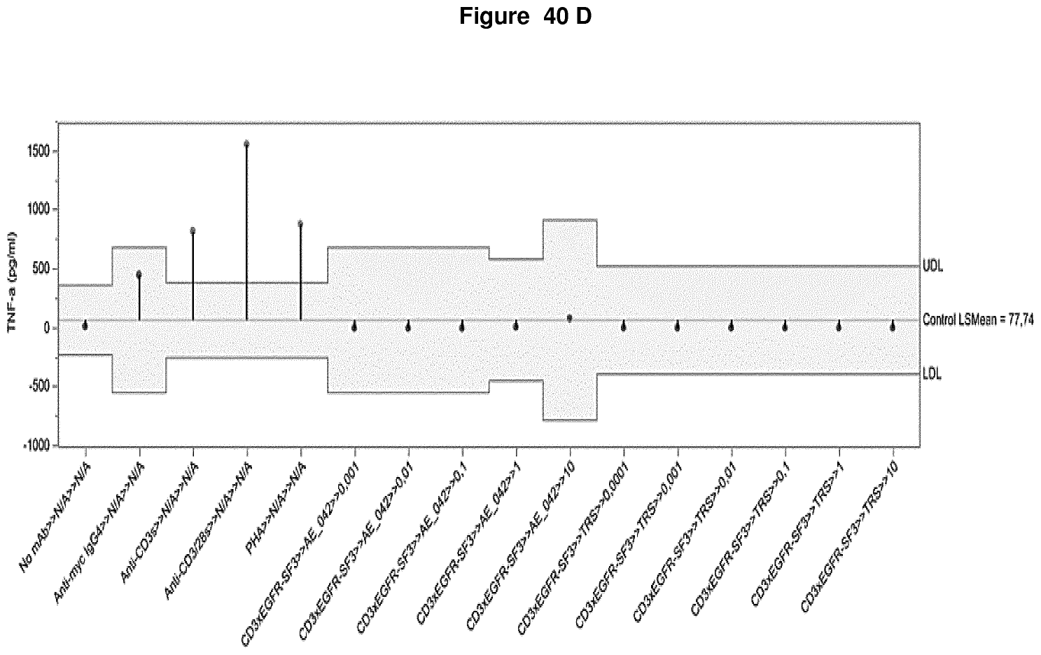

FIG. 40. Statistical comparison of the non-specific T cell cytokine responses between CD3×EGFR-SF3 and the isotype control in a high density PBMC assay. Data from FIG. 38 were analyzed by fit least square model followed by a Dunnett's comparison (α=0.05) to compare the means against the isotype control of IL-2 (A), IL-6 (B), IFN-γ (C), and TNF-α (D). Significant differences of the mean are shown as the needles bars outside of the decision limits (95% CI interval for each treatment; gray area).

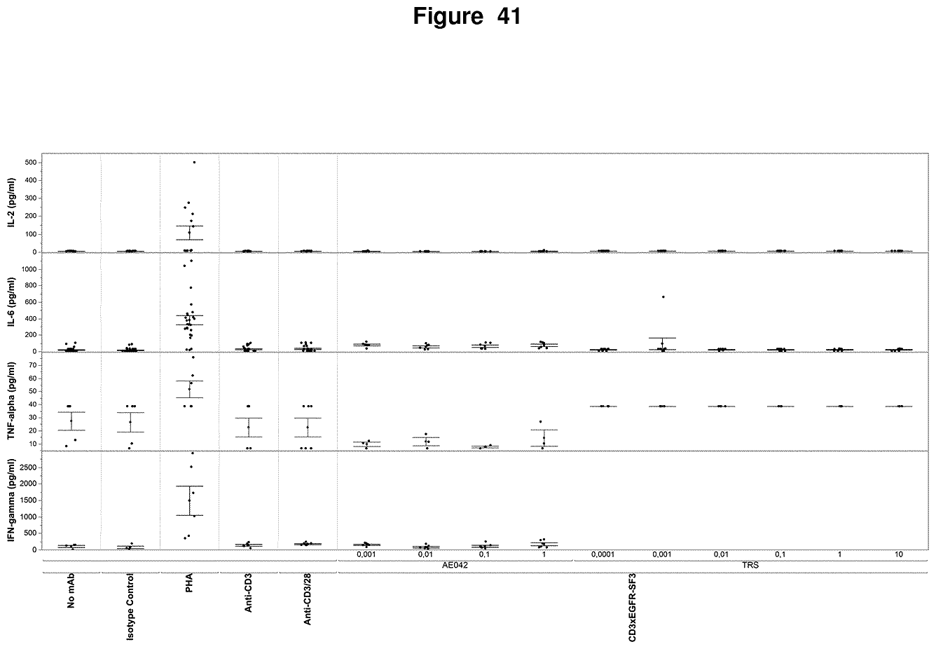

FIG. 41. CD3×EGFR-SF3 does not induce a cytokine response in a whole blood assay. Whole blood from healthy volunteers was cultured for 24 h in presence of increasing doses of CD3×EGFR-SF3 or controls and the levels of IL-2, IL-6, TNF-α, and IFN-γ were measured by Luminex in the serum. AE042 and TRS represent different batches of CD3×EGFR-SF3 and 0.001, 0.01, 0.1, and 1 the concentrations in ug/ml. The error bars represent means±SEM from four independent experiments.

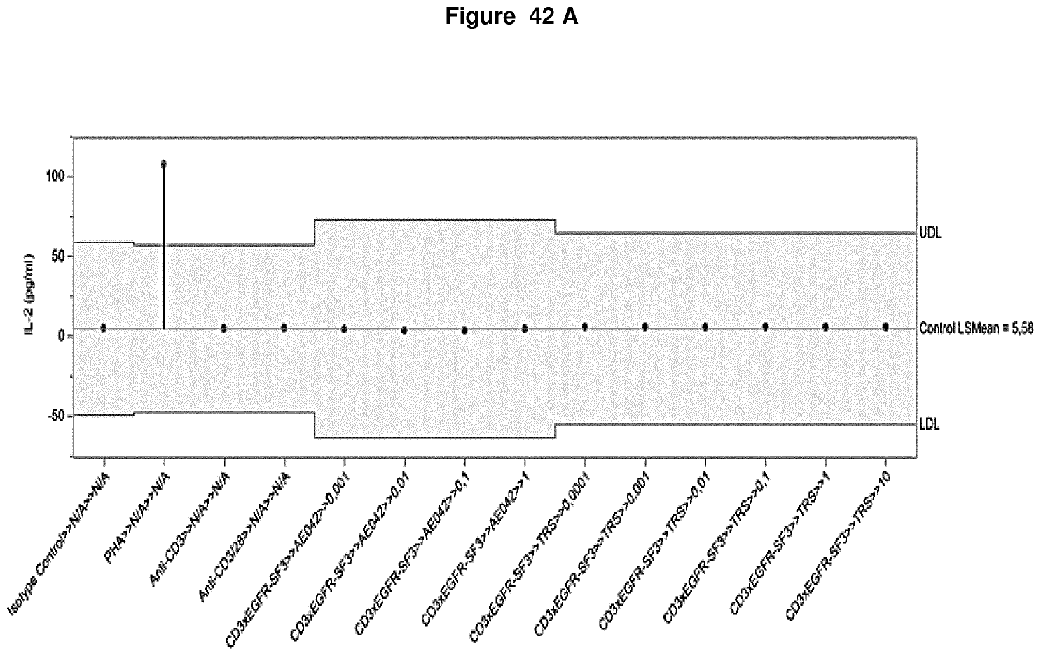

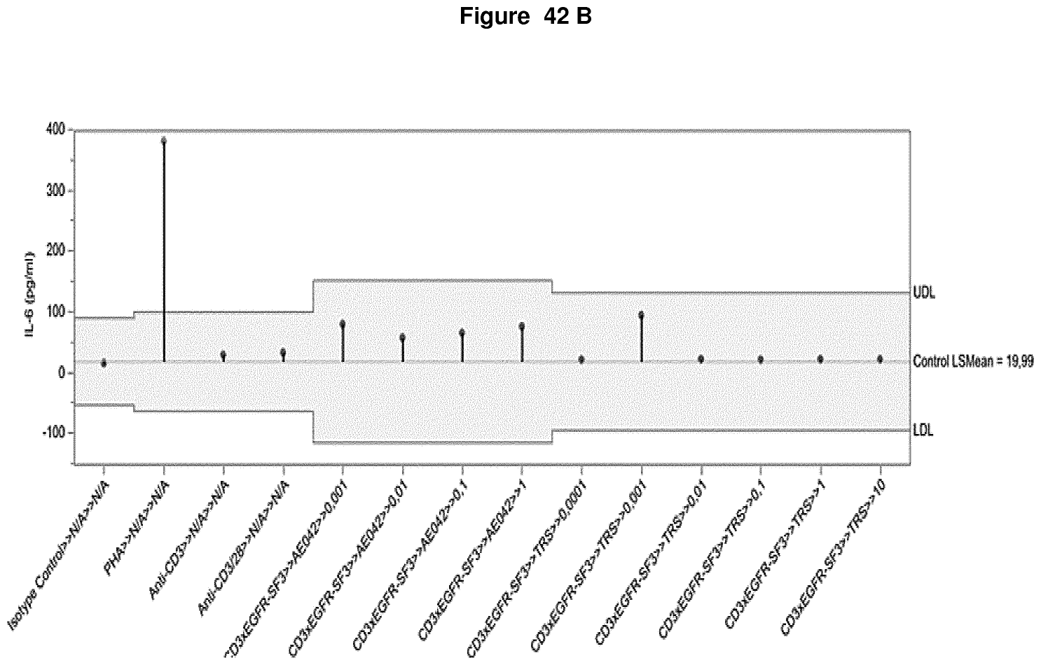

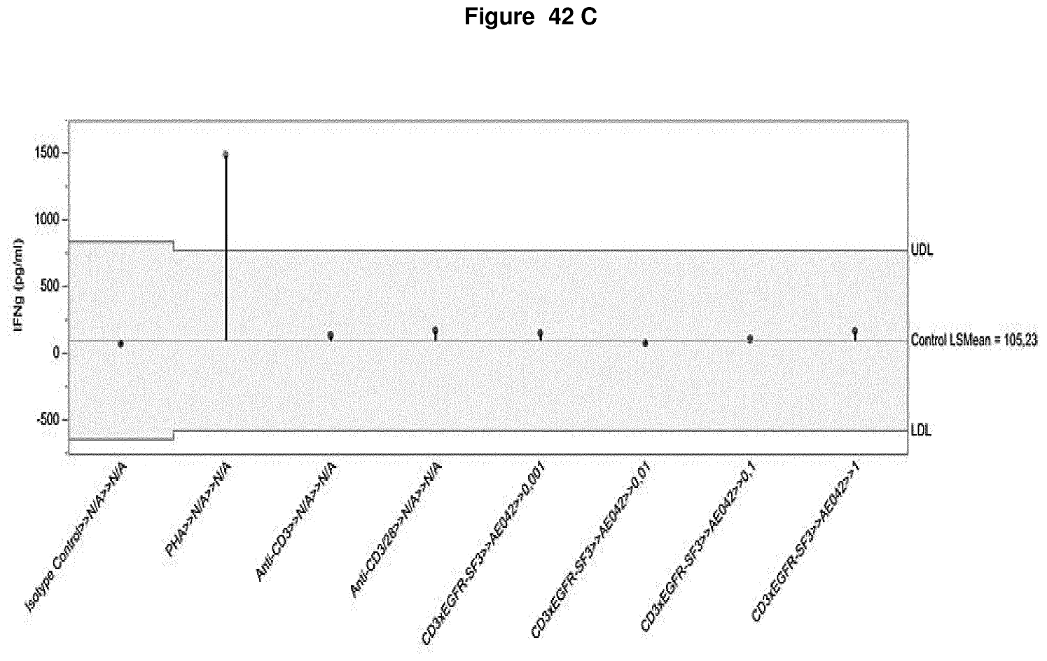

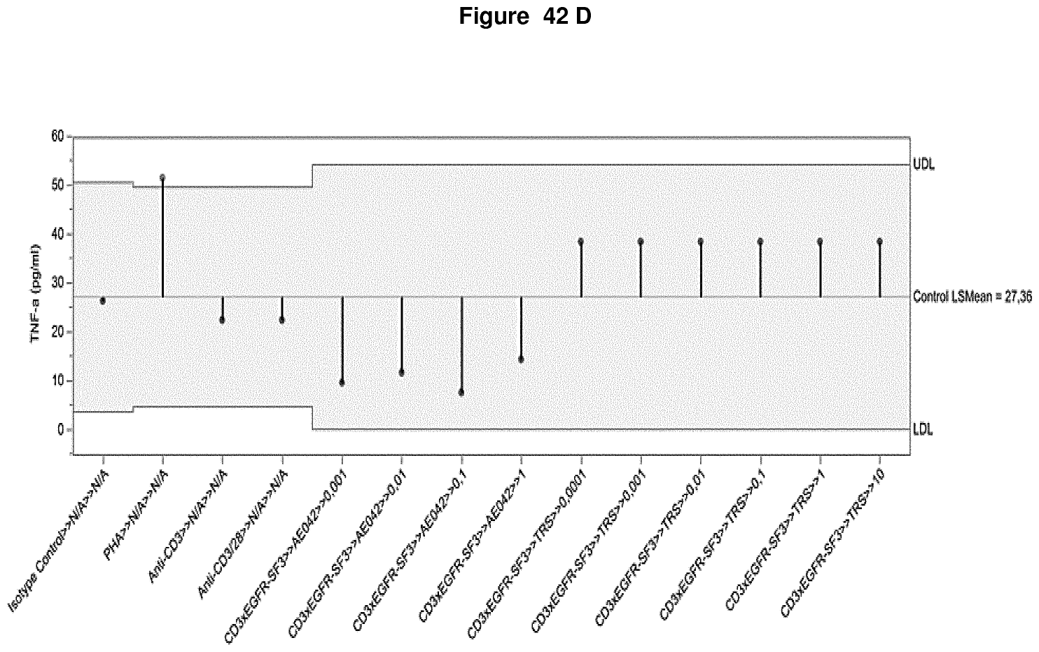

FIG. 42. Statistical comparison of the cytokine responses between CD3×EGFR-SF3 and the no mAb condition in a whole blood assay. Data from FIG. 41 were analyzed by fit least square model followed by a Dunnett's comparison (α=0.05) to compare the means against the no mAb control of IL-2 (A), IL-6 (B), IFN-γ (C), and TNF-α (D). Significant differences of the mean are shown as the needles bars outside of the decision limits (95% CI interval for each treatment; gray area).

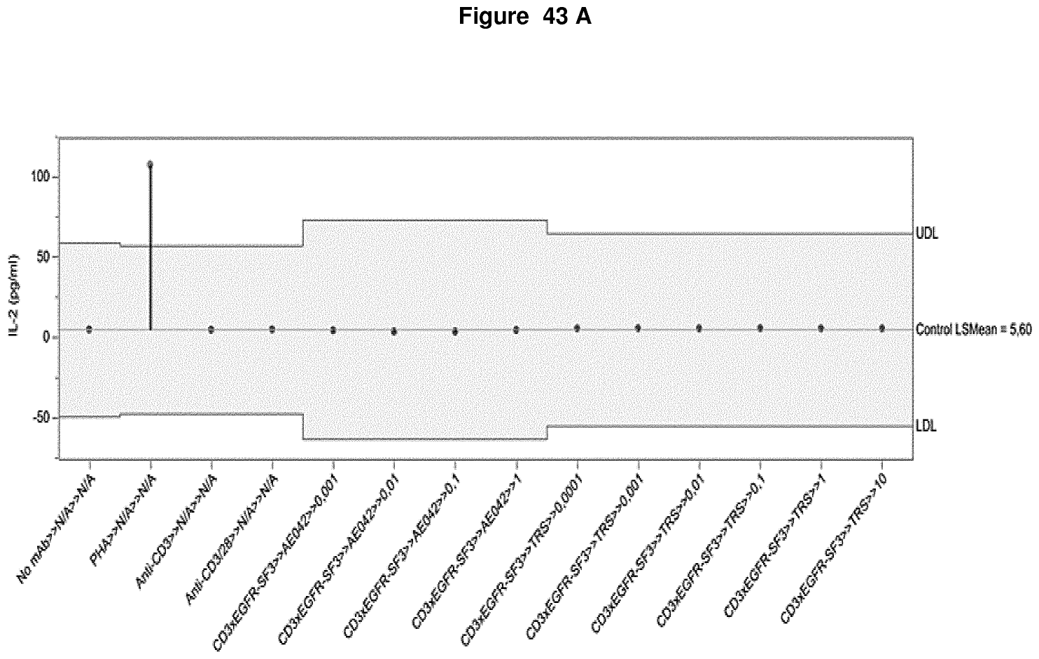

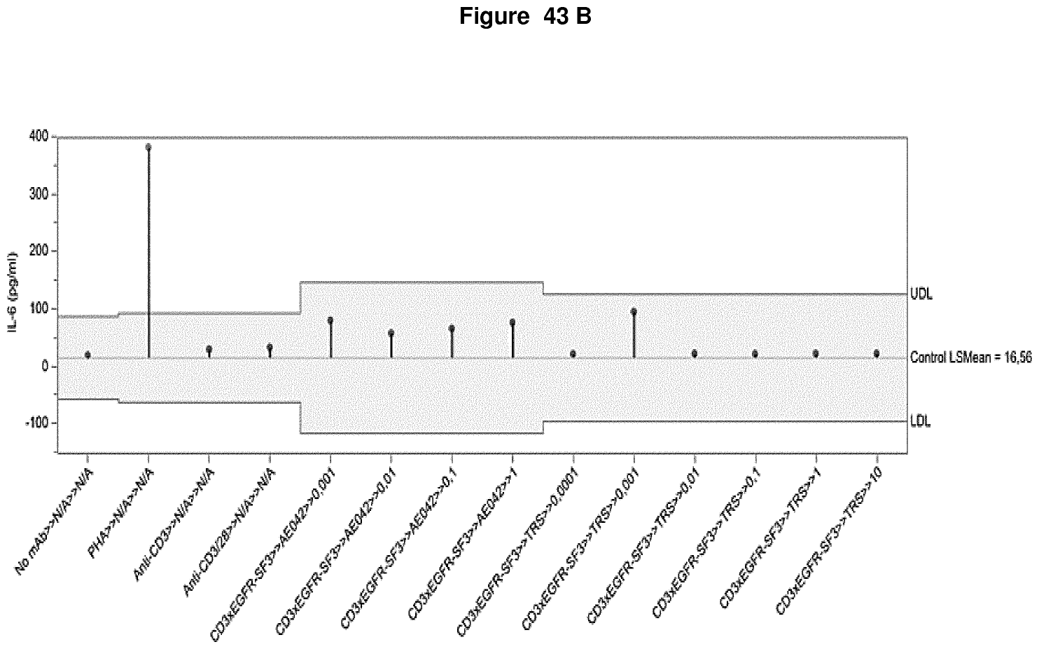

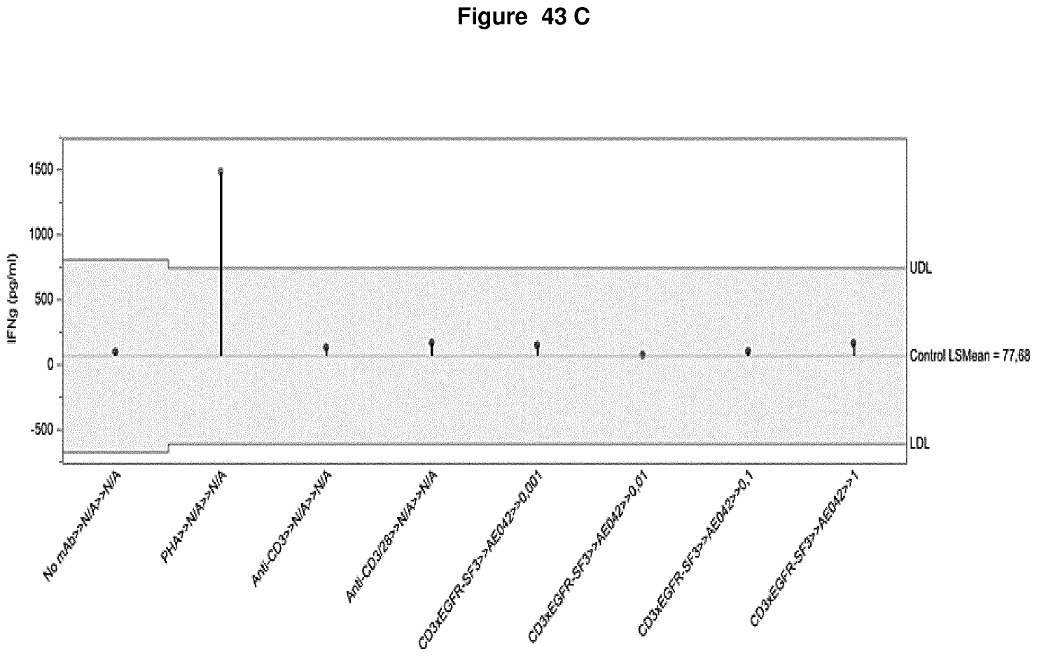

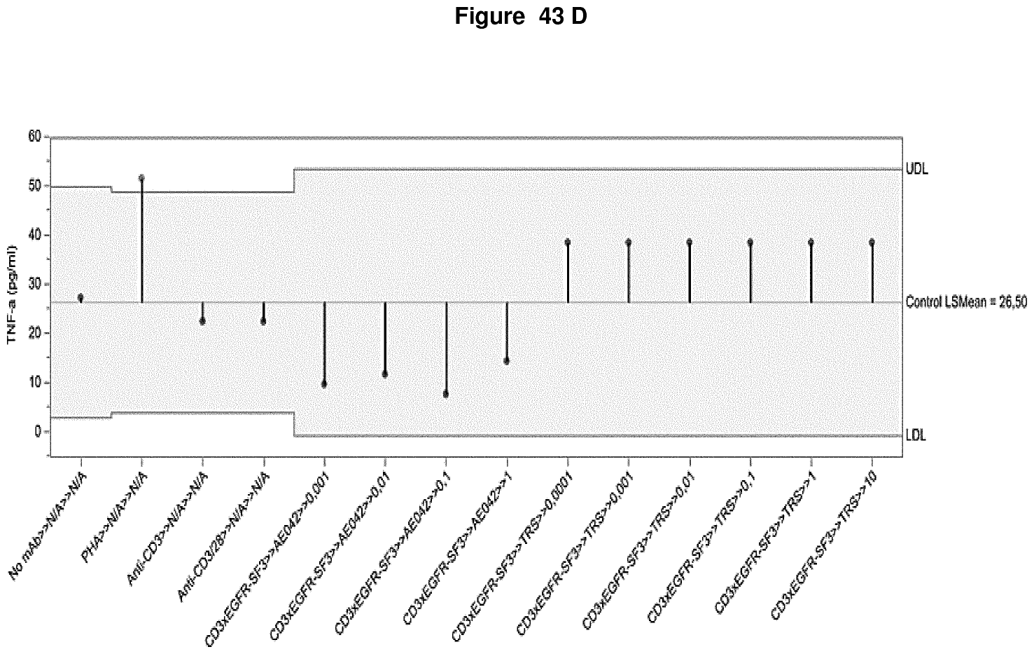

FIG. 43. Statistical comparison of the cytokine responses between CD3×EGFR-SF3 and the isotype control in a whole blood assay. Data from FIG. 41 were analyzed by fit least square model followed by a Dunnett's comparison (α=0.05) to compare the means against the isotype control of IL-2 (A), IL-6 (B), IFN-γ (C), and TNF-α (D). Significant differences of the mean are shown as the needles bars outside of the decision limits (95% CI interval for each treatment; gray area).

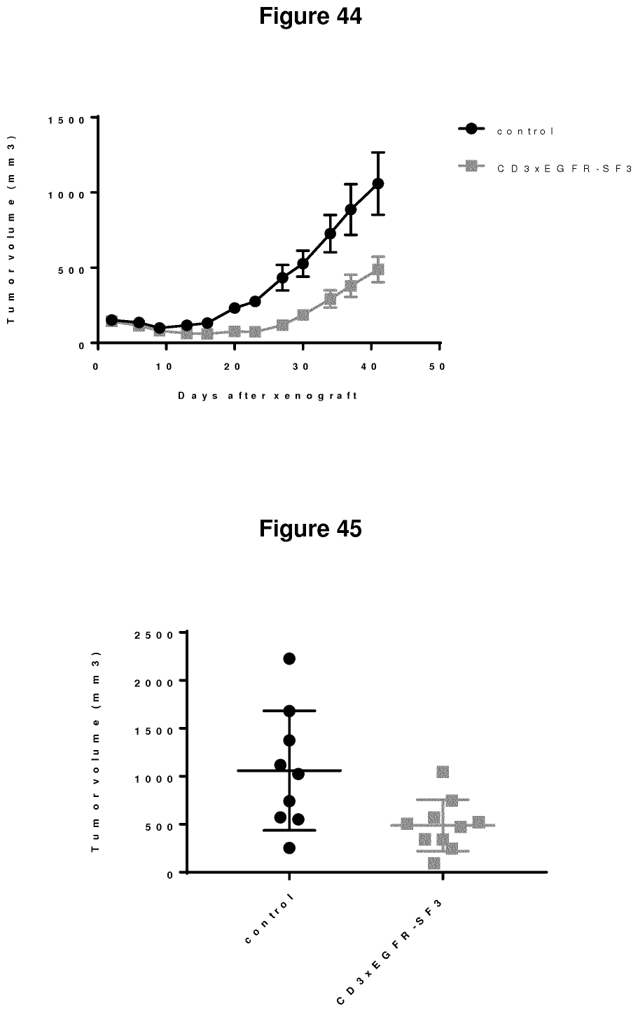

FIG. 44. Efficacy of CD3×EGFR-SF3 therapeutic treatment in NOD SCID xenografted mouse model. The expression level of EGFR on A549 cells was determined by sABC before the graph. A mix of tumor cells (target cells, T) and PBMCs (effector cells, E) were injected s.c. at an E:T ratio of 2:1 into the right flank area of NOD.CB17/AlhnRj-Prkdcscid/Rj (NOD/SCID) mice (n=4 to 5 per group per PBMC donor). CD3×EGFR-SF3 was administered i.v. at 2 mg/kg once a week starting on day 2 for 3 weeks. Tumor growth was determined by external caliper measurements. The graphs show the mean tumor size (in mm³)±SEM. 2 PBMC donors were included. Name of the study: A549_15.

FIG. 45. A549 tumor volume comparison between CD3×EGFR-SF3 in therapeutic treatment and control group at day 41. The data showed per group the tumor volume of each animal at day 41. Data are extracted from FIG. 44. Name of the study: A549_15.

Below is provided a set of non-exhaustive examples relating to the present invention.

### EXAMPLE 1: ENGINEERING OF CD3×EGFR BISPECIFIC ANTIBODIES IN DIFFERENT FORMATS

Hombach et al. (2007) demonstrated that the position of the targeted epitope within a target molecule has a major impact on the efficacy of T cell activation and that shortening the distance between the T-cell and the target cell membrane can increase the cytotoxic potential of a bispecific antibody. Our hypothesis was, that re-arranging the binding domains in a CD3×EGFR BEAT bispecific antibody could change the distance between the redirected T-cells and the EGFR expressing cancer cells and thus modulate the cytotoxic potential of our molecule.

The panitumumab anti-EGFR binder and the humanized SP34 anti-CD3 binder were engineered into a number of different BEAT formats as described below.

Construction of Alternative BEAT Architectures

Alternative BEAT architectures were designed by altering the position of the panitumumab anti-EGFR binder and the humanized SP34 anti-CD3 binder (FIG. 1). Depending on the architecture, the binders were formatted as single-chain fragment (scFv) or Fab. Binders in scFv format were fused via Gly4Ser or Gly4Thr linkers (SEQ ID NOs: 13 and 14) to confer flexibility. When a Fab was fused to a scFv, a Gly4Ser linker was added in between. Coding DNAs (cDNAs) encoding the different polypeptide chains in part or in full were first gene synthetized by GENEART AG (Regensburg, Germany) and modified using standard molecular biology techniques. PCR products were digested with appropriate DNA restriction enzymes, purified and ligated in a modified pcDNA3.1 plasmid (Invitrogen AG, Zug, Switzerland) carrying a CMV promoter and a bovine hormone poly-adenylation (poly(A)) previously digested with the same DNA restriction enzymes. All polypeptide chains were independently ligated in this expression vector where secretion was driven by the murine VJ2C leader peptide. Polypeptide chain A (see FIG. 1) generally contained in addition to the variable domain, an IgG1 hinge followed by an IgG3 CH2 domain with both L234A and L235A substitutions (EU numbering) and an IgG3 CH3 domain containing the BEAT (A) substitutions. To prevent Protein A binding due to the VH3 type framework of the humanized SP34 anti-CD3 binder, Protein A binding abrogating mutations N82aS and/or G65S were added when SP34 was placed on chain A. Polypeptide chain B generally contained an IgG1 hinge followed by an IgG1 CH2 domain with both L234A and L235A substitutions and an IgG1 CH3 domain containing the BEAT (B) substitutions. When a binder was present in Fab format, an IgG1 CH1 domain was also part of the polypeptide chain. The following molecules were constructed: CD3×EGFR_1 (SEQ ID NOs: 1, 2 and 3), CD3×EGFR_SF1 (SEQ ID NOs: 4, 5 and 6), CD3×EGFR_SF3 (SEQ ID NOs: 7, 2 and 8), CD3×EGFR_SF4 (SEQ ID NOs: 4, 5 and 9), CD3×EGFR_SD1 (SEQ ID NOs: 1, 2 and 10), CD3×EGFR_SD2 (SEQ ID NOs: 11, 10 and 2) and CD3×EGFR_9 (SEQ ID NOs: 1, 2 and 12).

Production of CD3×EGFR in Alternative BEAT Architectures

For transient expression, equal quantities of each engineered chain vector were co-transfected into suspension-adapted HEK293-EBNA cells (ATCC-LGL standards, Teddington, UK; Cat. No: CRL-10852) using polyethyleneimine (PEI; Sigma, Buchs, Switzerland). Typically, 100 ml of cells in suspension at a density of 0.8-1.2 million cells per ml are transfected with a DNA-PEI mixture. When recombinant expression vectors encoding the respective chains are introduced into the host cells, the immunoglobulin construct is produced by further culturing the cells for a period of 4 to 5 days to allow for secretion into the culture medium (EX-CELL 293, HEK293-serum-free medium (Sigma), supplemented with 0.1% pluronic acid and 4 mM glutamine). Cell-free culture supernatants containing the secreted proteins were prepared by centrifugation followed by sterile filtration. BEATs were then purified from cell-free supernatant using Protein A affinity resin (Repligen). Clarified supernatants were adjusted to pH 6.0 with NaH₂PO₄ at 0.2 M and loaded on Protein A by gravity flow. Columns were washed with 20 CV of 0.2 M citrate phosphate buffer pH 6.0. Proteins were eluted with 16 CV of 20 mM sodium acetate at pH 4.1 and neutralized with 0.1 volume of 1 M Tris pH 8.0 (Sigma). Samples were buffer exchanged into PBS pH 7.4 using Illustra NAP-10 columns (GE Healthcare). Exceptionally, CD3×EGFR_SF3 cell-free supernatants were loaded onto a 1 ml HiTrap™ MabSelect SuRe™ Protein A column pre-equilibrated in 0.2 M citrate phosphate buffer pH 6.0 and operated on an ÄKTApurifier™ chromatography system (both from GE Healthcare Europe GmbH; column Cat. No: 11-0034-93) at a flow rate of 1 ml/min. Running buffer was 0.2 M citrate phosphate buffer pH 6.0. Washing buffer was 0.2 M citrate phosphate buffer pH 5.0. Elution was performed using 20 mM sodium acetate buffer pH 4.1. Elution was followed by OD reading at 280 nm; fractions containing CD3×EGFR antibodies were pooled and neutralized with 0.1% volume of 1 M Tris pH 8.0. Samples were buffer exchanged into PBS pH 7.4 using Illustra NAP-10 columns (GE Healthcare Europe GmbH, Glattbrugg, Switzerland).

### EXAMPLE 2: GENERATION AND CHARACTERIZATION OF ANTI-EGFR ANTIBODIES SPECIFIC FOR DOMAIN IV OF EGFR

Immunization

Female BALB/c mice, 7 weeks of age (Harlan) were used to generate antibodies against the extracellular domain 4 of EGFR. The mice were immunized three times by the intraperitoneal (i.p) and the subcutaneous (s.c.) routes with a mixture of either 50 μg human EGFR His-tagged protein (SEQ ID NO: 15) or 50 μg extracellular domain IV EGFR His-tagged protein (SEQ ID NO: 16), in combination with 100 μl of adjuvant. The presence of circulating anti-EGFR antibodies specific to domain IV in the immunized mouse sera was evaluated by direct ELISA using plates coated with the recombinant human EGFR his or domain IV His proteins. Mouse sera were serially diluted (from 1:100 to 1:109) and added to 96-well ELISA plates and the bound antibodies were detected using a goat anti-mouse molecule-HRP (Jackson Immunoresearch). A final intravenous boost with 10 μg of antigen without adjuvant was performed in animals displaying the best anti-EGFR domain IV IgG serum titer three days before sacrifice. Animals were euthanized and the spleens were harvested for fusion.

Fusion Protocol

1 ml of warm PEG1500 was slowly added to the cell slurry over the course of 1 min while swirling. The cells were gently mixed for a further 2 minutes. 4 ml of warm SFM was then added over a period of 4 min. 10 ml of warm SFM was slowly added and the cells were incubated for 5 min in a water bath at 37° C. The cells were centrifuged at 1000 rpm for 5 min and re-suspended in 200 ml of complete medium. For the fusion, the cells were plated at 200 μl/well in ten 96 flat-bottom well plates.

Screening of Hybridoma Supernatants on Membrane-Bound EGFR by Flow Cytometry

Approximately 1900 wells from two fusions were analyzed by ELISA for their content in murine IgG specific for recombinant human EGFR. Positive hybridoma supernatants were further screened against recombinant domain IV of human EGFR, immobilized on 96-well ELISA plates. Among all the tested clones, two parental candidates, 3A6 and 10E6, were identified and tested by flow cytometry on BAF cells transfected with membrane-bound EGFR. In this assay, 105 cells/well were incubated with 100 ul of supernatants at 4° C. for 1 hour. Following this primary incubation, cells were centrifuged at 1300 rpm, 2 min and pellets were resuspended with 100 ul of PE-labeled goat anti-mouse secondary antibody diluted at 1/100 in FACS buffer. Cells were then incubated for 30 minutes at 4° C. and washed twice, the supernatants removed and the cells resuspended in 150 μl of FACS buffer. The samples were analyzed by flow cytometry. As shown in FIG. 2, the results from the flow cytometry experiment show that both 3A6 and 10E6 hybridoma candidates recognize the membrane-bound human EGFR receptor expressed on BAF cells, compared to the isotype control used at 10 ug/ml. These two hybridoma candidates were expanded and subcloned.

Screening of Hybridoma Supernatants on Soluble EGFR by ELISA

The subclones 3A6A12B5 and 10E6F5 were derived from 3A6 and 10E6 parental clones, respectively. Supernatants from both subclones were harvested and purified using a LC-kappa mouse affinity matrix (Life technologies), according to the manufacturer's instructions. These purified antibodies were tested by ELISA on 96-well plates coated with either soluble human EGFR or recombinant EGFR-Her3 chimeric constructs. These molecules were diluted at 2 ug/ml in PBS and immobilized overnight at 4° C. on a high binding 96-well plates. The plates were blocked with PBS 2% Bovine Serum Albumin (BSA) and incubated for 1 hour with a serial dilution of either 3A6A12B5 or 10E6F5. As a control, Panitumumab (Vectibix®) was used at the same concentrations. The plates were then washed with PBS 0.01% Tween and incubated for 1 hour with 100 ul of either goat anti-mouse IgG (to detect 3A6A12B5 and 10E6F5) (Jackson ImmunoResearch Europe Ltd, Newmarket, UK) or goat anti-human IgG, F(ab′)2 fragment specific-HRP (to detect Panitumumab). Following this incubation, the plates were washed and incubated with 100 ul of TMB substrate. The reaction was stopped by adding 100 μl of H₂SO₄ 2N and the absorbance was read at 450 nm on a Synergy HT2 spectrophotometer (Biotek, USA; distributor: WITTEC AG, Littau, Switzerland). Results from FIG. 3 show that purified 3A6A12B5 and 10E6F5 antibodies recognize in a dose dependent manner the soluble EGFR molecule (A) and bind also the chimeric Her3I-III-EGFR IV (C) and EGFR IV (D) molecules. Conversely, none of these two candidates recognize the chimeric EGFR I-III Her3 IV molecule (B). These results show that both 3A6A12B5 and 10E6F5 antibodies bind EGFR, and specifically to domain IV.

Redirected Lysis Assay (RDL)

This assay was performed following the procedure described in the example. FIG. 4 shows that both BEAT CD3-EGFR_5 and CD3-EGFR_8 molecules display a killing potential against EGFR+A549 target cells.

### EXAMPLE 3: HUMANIZATION OF ANTI-EGFR DOMAIN IV ANTIBODIES 3A6 AND 10E6

3.1 Human EGFR and Chimeric Human EGFR-ErbB3 Proteins Used for Mouse Immunization and Antibody Characterization

Coding DNA (cDNA) encoding the polypeptide chain of human EGFR soluble extracellular region (UniProt accession No: P00533 residues 25-638, referred to herein as hEGFR, SEQ ID NO: 15) with a C-terminal poly-histidine tag was synthetized by GENEART AG (Regensburg, Germany) and modified using standard molecular biology techniques. PCR products were digested with appropriate DNA restriction enzymes, purified and ligated in a modified pcDNA3.1 plasmid carrying a CMV promoter and a bovine hormone poly-adenylation (poly(A)) signal previously digested with the same DNA restriction enzymes.

The following EGFR domain IV only constructs were PCR amplified from the hEGFR construct described above and restriction ligated into the expression plasmid mentioned above: hEGFR-IV_505-638 (505-638 indicates residue range, this construct additionally carried the mutation W516A to increase solubility, mutation was added by standard overlapping PCR using primers including the appropriate mutation), hEGFR-IV_556-638 and hEGFR-IV_580-638 (SEQ ID NO: 16, 17 and 18). Cynomolgus EGFR-IV_556-638 and 580-638 (herein referred to as cEGFR-IV_556-638 and 580-638) were generated by adding the mutations A566V (only for the construct encompassing residues 556-638), P637A and T638R to the human construct by overlapping PCR (SEQ ID NO: 19 and 20).

The following chimeric human EGFR-ErbB3 constructs (human ErbB3, UniProt accession No: P21860) were designed and cDNA encoding their polypeptide chains were synthesized by Eurofins Genomics: hEGFR-I-II-III_hErbB3-IV and hErbB3-I-II-III_hEGFR-IV (SEQ ID NO: 21 and 22).

For transient expression of the above described EGFR constructs, the appropriate expression vectors were transfected into suspension-adapted HEK-EBNA cells (ATCC-CRL-10852) using polyethyleneimine (PEI). Typically, 100 ml of cells in suspension at a density of 0.8-1.2 million cells per ml are transfected with a DNA-PEI mixture containing 100 μg of expression vector. When recombinant expression vectors are introduced into the host cells, proteins are produced by further culturing the cells for a period of 4 to 5 days to allow for secretion into the culture medium (EX-CELL 293, HEK293-serum-free medium; Sigma, Buchs, Switzerland), supplemented with 0.1% pluronic acid, 4 mM glutamine). EGFR was then purified from cell-free supernatant using Ni sepharose Excel (GE Healthcare Europe GmbH, Glattbrugg, Switzerland) and used for further analysis.

3.2 Chimeric 3A6 and 10E6

Using standard molecular biology techniques, VH and VL sequences extracted from antibodies from hybridoma cells were re-formatted into mouse-human chimeric IgG1 antibodies. Mouse 3A6 and 10E6 VH domains were fused to human IgG1 Fc (CH1-hinge-CH2-CH3) and the corresponding VL domains were fused to IgG1 constant kappa. The resulting constructs were ligated into the modified pcDNA3.1 plasmid described above (3A6 chimeric antibody SEQ ID NO: 23 and 24, 10E6 chimeric antibody SEQ ID NO: 25 and 26).

For transient expression of the chimeric antibodies, the recombinant vectors for the heavy- and the light chain were transfected at a 1:1 molar ratio into suspension-adapted HEK-EBNA cells using PEI. Antibodies were then purified from cell-free supernatant using Protein A affinity resin (Repligen, Waltham Mass., USA). Clarified supernatants were loaded on Protein A by gravity flow. Proteins were eluted with 0.1 M glycine pH 3.0. Samples were buffer exchanged into PBS pH 7.4 using Illustra NAP-10 columns (GE Healthcare Europe GmbH, Glattbrugg, Switzerland).

3.3 Humanization of Mouse Monoclonal 3A6

Humanization of the anti-human EGFR mouse antibody 3A6 including selection of human acceptor frameworks that substantially retain the binding properties of human CDR-grafted acceptor frameworks is described herein. Two grafts were prepared, one using the best-fit framework and another using a stable framework.

Homology matching was used to choose human best-fit acceptor frameworks to graft 3A6 CDRs. Databases e.g. a database of germline variable genes from the immunoglobulin loci of human and mouse (the IMGT database, supra) or the VBASE2 (Retter I et al, (2005) Nucleic Acids Res. 33, Database issue D671-D674) or the Kabat database (Johnson G et al, (2000) Nucleic Acids Res. 28: 214-218) or publications (e.g., Kabat E A et al, supra) may be used to identify the human subfamilies to which the murine heavy and light chain V regions belong and determine the best-fit human germline framework to use as the acceptor molecule. Selection of heavy and light chain variable sequences (VH and VL) within these subfamilies to be used as acceptor may be based upon sequence homology and/or a match of structure of the CDR1 and CDR2 regions to help preserve the appropriate relative presentation of the six CDRs after grafting.

For example, use of the IMGT database indicates good homology between the 3A6 heavy chain variable domain framework and the members of the human heavy chain variable domain subfamily 4. Highest homology and identity of both CDRs and framework sequences were observed for germline sequence IGHV4-4*08 (SEQ ID NO: 27) which had sequence identity of 59.4% for the whole sequence up to CDR3.

Using the same approach, 3A6 light chain variable domain sequence showed good homology to the members of the human light chain variable domain kappa subfamily 6. Highest homology and identity of both CDRs and framework sequences were observed for germline sequence IGKV6-21*02 (SEQ ID NO: 28) with sequence identity of 69.5%.

Selection of human heavy and light chain variable sequences (VH and VL) to be used as acceptor may be based upon germlines with good biophysical properties (as documented in Ewert S et al., (2003) J. Mol. Biol, 325, 531-553) and/or pairing as found in natural antibody repertoire (as documented in Glanville J et al., (1999) Proc Natl Acad Sci USA, 106(48):20216-21; DeKosky B J et al., (2015) Nat Med, 21(1):86-91). Framework sequences known in the field for good paring and/or stability are the human IGHV3-23*04 (SEQ ID NO: 29) and IGKV1-39*01 (SEQ ID NO: 30) frameworks which were used as acceptor frameworks for the 3A6 stable graft humanization.

Two humanized antibodies of human gamma one isotype were prepared. The antibodies encompassed a human-mouse hybrid heavy chain variable domain and a human-mouse hybrid light chain variable domain. The first hybrid heavy chain variable domain was based on the human heavy chain variable domain IGHV4-4*08 wherein germline CDRH1 and H2 where respectively replaced for 3A6 CDRH1 and CDRH2. Best matching JH segment sequence to the human acceptor framework was identified from the IMGT database using homology search. To accommodate CDRs on to the human acceptor framework key positions were modified by substituting human residues to mouse residues. This process is called back-mutation and is the most unpredictable procedure in the humanization of monoclonal antibodies. It necessitates the identification and the selection of critical framework residues from the mouse antibody that need to be retained in order to preserve affinity while at the same time minimizing potential immunogenicity in the humanized antibody. The resulting human-mouse hybrid heavy chain variable sequence having human IGHV4-4*08 framework regions, 3A6 mouse CDRs, key human to mouse framework back-mutations and best matching JH to human acceptor is referred herein as heavy chain variable domain 3A6-best-fit-VH with SEQ ID NO: 31. The second hybrid heavy chain variable domain was based on the human heavy chain variable domain IGHV3-23*04 wherein germline CDRH1 and H2 where respectively replaced for 3A6 CDRH1 and CDRH2. Best matching JH segment sequence to the human acceptor framework was identified from the IMGT database using homology search. The resulting human-mouse hybrid heavy chain variable sequence having human IGHV3-23*04 framework regions, 3A6 mouse CDRs, key human to mouse framework back-mutations and best matching JH to human acceptor is referred herein as heavy chain variable domain 3A6-stable-VH with SEQ ID NO: 32.

Similarly, the first human-mouse hybrid light chain variable domain used for this first humanized antibody candidate had human IGKV6-21*02 framework regions, 3A6 mouse CDRs and best matching JK to human acceptor, and is referred herein as light chain variable domain 3A6-best-fit-VL with SEQ ID NO: 33 (no back-mutations in the framework were required in this case as all key positions were the same in the mouse and human framework). The first humanized antibody encompassing 3A6-best-fit-VH and 3A6-best-fit-VL is abbreviated herein as 3A6-best-fit antibody. The second human-mouse hybrid light chain variable domain used for the second humanized antibody candidate had human IGKV1-39*01 framework regions, 3A6 mouse CDRs, key human to mouse framework back-mutations and best matching JK to human acceptor, and is referred herein as light chain variable domain 3A6-stable-VL with SEQ ID NO: 34. The second humanized antibody encompassing 3A6-stable-VH and 3A6-stable-VL is abbreviated herein as 3A6-stable antibody.

3.4 Humanization of Mouse Monoclonal 10E6

Humanization of the anti-human EGFR mouse antibody 10E6 including selection of human acceptor frameworks that substantially retain the binding properties of human CDR-grafted acceptor frameworks is described herein. Two grafts were prepared, one using the best-fit framework and another using a most stable framework.

Homology matching was used as described above to choose human best-fit acceptor frameworks to graft 10E6 CDRs. The IMGT database indicates good homology between the 10E6 heavy chain variable domain framework and the members of the human heavy chain variable domain subfamily 4. Highest homology and identity of both CDRs and framework sequences were observed for germline sequence IGHV4-30-4*01 (SEQ ID NO: 35) which had sequence identity of 73.2% for the whole sequence up to CDR3.

Using the same approach, 10E6 light chain variable domain sequence showed good homology to the members of the human light chain variable domain kappa subfamily 6. Highest homology and identity of both CDRs and framework sequences were observed for germline sequence IGKV6-21*02 (SEQ ID NO: 14) with sequence identity of 69.5%.

Stable frameworks were chosen as described above. Human IGHV3-23*04 (SEQ ID NO: 29) and IGKV1-39*01 (SEQ ID NO: 30) were used as acceptor frameworks for the stable graft humanization.

Two humanized antibodies of human gamma one isotype were prepared. The antibodies encompassed a human-mouse hybrid heavy chain variable domain and a human-mouse hybrid light chain variable domain. The first hybrid heavy chain variable domain was based on the human heavy chain variable domain IGHV4-30-4*01 wherein germline CDRH1 and H2 where respectively replaced for 10E6 CDRH1 and CDRH2. Best matching JH segment sequence to the human acceptor framework was identified from the IMGT database using homology search. The resulting human-mouse hybrid heavy chain variable sequence having human IGHV4-30-4*01 framework regions, 10E6 mouse CDRs, key human to mouse framework back-mutations and best matching JH to human acceptor is referred herein as heavy chain variable domain 10E6-best-fit-VH with SEQ ID NO: 36. The second hybrid heavy chain variable domain was based on the human heavy chain variable domain IGHV3-23*04 wherein germline CDRH1 and H2 where respectively replaced for 10E6 CDRH1 and CDRH2. Best matching JH segment sequence to the human acceptor framework was identified from the IMGT database using homology search. The resulting human-mouse hybrid heavy chain variable sequence having human IGHV3-23*04 framework regions, 10E6 mouse CDRs, key human to mouse framework back-mutations and best matching JH to human acceptor is referred herein as heavy chain variable domain 10E6-stable-VH with SEQ ID NO: 37.

Similarly, the first human-mouse hybrid light chain variable domain used for this first humanized antibody candidate had human IGKV6-21*02 framework regions, 10E6 mouse CDRs, key human to mouse framework back-mutations and best matching JK to human acceptor, and is referred herein as light chain variable domain 10E6-best-fit-VL with SEQ ID NO: 38. The first humanized antibody encompassing 10E6-best-fit-VH and 10E6-best-fit-VL is abbreviated herein as 10E6-best-fit antibody. The second human-mouse hybrid light chain variable domain used for the second humanized antibody candidate had human IGKV1-39*01 framework regions, 10E6 mouse CDRs, key human to mouse framework back-mutations and best matching JK to human acceptor, and is referred herein as light chain variable domain 10E6-stable-VL with SEQ ID NO: 39. The second humanized antibody encompassing 10E6-stable-VH and 10E6-stable-VL is abbreviated herein as 10E6-stable antibody.

3.5 Production of Humanized 3A6 and 10E6 Antibodies

Coding DNA sequences (cDNAs) for best-fit VH and VL and stable VH and VL were synthesized by Eurofins Genomics (Ebersberg, Germany) and modified using standard molecular biology techniques. VH domains were fused to a human IgG1 CH1-hinge-CH2-CH3 portion and restriction ligated into the expression vector described above. Similarly, genes for the VL domains were fused to the human constant kappa domain and ligated into a separate expression vector. The resulting antibodies were 3A6-best-fit (SEQ ID NO: 40 and 41), 3A6-stable (SEQ ID NO: 42 and 43), 10E6-best-fit (SEQ ID NO: 44 and 45) and 10E6-stable (SEQ ID NO: 46 and 47).

For transient expression of antibodies, the recombinant vectors for the heavy- and the light chain were transfected at a 1:1 molar ratio into suspension-adapted HEK-EBNA cells using PEI. Antibodies were then purified from cell-free supernatant using Protein A affinity resin. Clarified supernatants were loaded on Protein A by gravity flow. Proteins were eluted with 0.1 M glycine pH 3.0. Samples were buffer exchanged into PBS pH 7.4 using Illustra NAP-10 columns.

3.6 Kinetic Binding Affinity Constants of the Chimeric and Humanized Antibodies for Human EGFR by Surface Plasmon Resonance (SPR)

Kinetic binding affinity constants (KD) were measured. Measurements were conducted on a BIAcore T200 (GE Healthcare—BIAcore, Uppsala, Sweden) at room temperature, and analyzed with the Biacore T200 Evaluation software. A Series S CM5 sensor chip was covalently coupled with Protein G and 100 RUs of antibody of interest was captured. hEGFR was injected at concentrations ranging from 19.53-2500 nM for 3A6 and 2.4-312.5 nM or 1-250 nM for 10E6 based antibodies at a flow rate of 30 μl/min in HBS-EP+ buffer (FIGS. 5 and 6, data are expressed as number of response units (abbreviated RU; Y axis) vs. time (X axis)). After each binding event, the surface was regenerated with 10 μl of glycine buffer pH 1.5. Dissociation time was 4 min. Experimental data were processed using a 1:1 Langmuir model with global Rmax.

3.7 Production of 3A6 and 10E6 Bispecific Antibodies in Combination with SP34

In order to generate bispecific BEAT antibodies combining 3A6 and 10E6 binders with humanized SP34, the VH fragments mentioned above were fused to a human IgG1 CH1-hinge followed by an IgG1 CH2 and an IgG1 CH3 domain containing the BEAT (A) substitutions. The CH2 domain contained both L234A and L235A substitutions (EU numbering). In-house humanized SP34 in scFv format followed by a short five amino acid linker (Gly4Thr) was fused to a human IgG3 CH2 followed by an IgG3 CH3 domain containing the BEAT (B) substitutions. The CH2 domain contained both L234A and L235A substitutions. Constructs were ligated into the expression vector as described above. The resulting molecules were CD3×EGFR_5 (SEQ ID NO: 48, 24 and 49), CD3×EGFR_6 (SEQ ID NO: 50, 26 and 49), CD3×EGFR_7 (SEQ ID NO: 48, 35 and 49) and CD3×EGFR_8 (SEQ ID NO: 52, 36 and 47).

For transient expression of the BEAT antibodies, the recombinant vectors for the heavy- and the light chain and the scFv-Fc chain were transfected at a 1:1:1 molar ratio into suspension-adapted HEK-EBNA cells using PEI as described above. Cell-free supernatants were loaded onto a 1 ml HiTrap™ MabSelect SuRe™ Protein A column pre-equilibrated in 0.2 M citrate phosphate buffer pH 6.0 and operated on an ÄKTApurifier™ chromatography system (both from GE Healthcare Europe GmbH; column Cat. No: 11-0034-93) at a flow rate of 1 ml/min. Running buffer was 0.2 M citrate phosphate buffer pH 6.0. Washing buffer was 0.2 M citrate phosphate buffer pH 5.0. Elution was performed using 20 mM sodium acetate buffer pH 4.1. Elution was followed by OD reading at 280 nm; fractions containing CD3×EGFR antibodies were pooled and neutralized with 0.1% volume of 1 M Tris pH 8.0.

3.8 Epitope Mapping and Cross-Reactivity with Cynomolgus Monkey

Surface plasmon resonance was used to evaluate the binding of 3A6 and 10E6 antibodies to various human and cynomolgus EGFR domain IV constructs with the aim to narrow down the epitope within EGFR domain IV and to demonstrate cross-reactivity with cynomolgus EGFR.

Measurements were performed on a Biacore 2000 instrument (GE). A CM5 sensor chip was covalently coupled with Protein G and 100 RUs of 3A6 or 10E6 chimeric antibody were captured. Various human and cynomolgus EGFR constructs were injected as analyte at a concentration of 200 nM at a flow rate of 30 μl/min in HBS-EP buffer. Dissociation time was 4 min. After each binding event, the surface was regenerated with 10 μl of glycine buffer pH 1.5. To verify the integrity of the EGFR domain IV constructs, a polyclonal goat anti-EGFR known to us to contain at least one anti EGFR domain IV binder (AF231, Bio-Techne AG, Zug, Switzerland) was immobilized as described above and human and cynomolgus EGFR constructs were injected as described above. Results are shown in FIG. 7. Both, 3A6 and 10E6 antibodies were able to bind human and cynomolgus EGFR-IV_556-638. EGFR-IV_580-638 was not bound by either of the antibodies. Thus we conclude that the binding epitope of 3A6 and 10E6 antibodies is within EGFR domain IV and more precisely within residues 556-638. Furthermore, residues 556-580 contain a required part of the epitope. Additionally, we demonstrate that both, 3A6 and 10E6 antibodies are cross-reactive to cynomolgus EGFR.

3.9 Thermostability of 10E6-Best-Fit and 10E6-Stable Antibodies

The thermal stability of 10E6-best-fit and 10E6-stable antibodies in PBS buffer was investigated by differential scanning calorimetry (DSC). Calorimetric measurements were carried out on a VP-DSC Capillary differential scanning microcalorimeter (Malvern Instruments Ltd, Malvern, UK). The cell volume was 0.128 ml, the heating rate was 1° C./min and the excess pressure was kept at 64 p.s.i. All samples were used at a concentration of 1 mg/ml in PBS (pH 7.4). The molar heat capacity of each protein was estimated by comparison with duplicate samples containing identical buffer from which the protein had been omitted. The partial molar heat capacities and melting curves were analyzed using standard procedures. Thermograms were baseline-corrected, concentration normalized, and further analyzed using a Non-Two State model using the Origin v7.0 software (supplied by Malvern Instruments Ltd).

Results are shown in FIG. 8. Melting temperatures (Tm) for 10E6-best-fit Fab and 10E6-stable Fab were determined and were 82.8° C. and 84.9° C. respectively.

### EXAMPLE 4: CD3-EGFR_1 EFFICACY IN A549 TUMORS XENOGRAFTED IN S.C

Material and Methods

4.1. Cell Line Culture Conditions

Cells were cultured in standard media in a humidified atmosphere of 5% CO₂ at 37° C. The cells were passaged 2 to 3 times per week to maintain them at optimal confluency and were routinely tested for mycoplasma contamination using the MycoAlert detection kit (LT07-318, Lonza). The cells consistently tested mycoplasma contamination free.

4.2. Effector Cells: Human Peripheral Blood Mononuclear Cells (PBMC)

Peripheral blood mononuclear cells (PMBCs) were harvested from blood filters obtained from La Chaux-de-Fonds Transfusion Center. For blood filter processing, 40 ml of PBS supplemented with 1% Liquemine® (Drossapharm) was injected into the blood filter and the solution containing the PBMCs was collected in 50 ml blood separation tubes (Chemie Brunschwig, PAA535710) previously loaded with ficoll (GE Healthcare, 17-1440-03). The ficoll tubes were centrifuged for 20 min at 800 g at room temperature (RT) without the brake and the PBMC “buffy coat” ring was harvested and transferred into 50 ml falcon tubes containing 30 ml of PBS. The PBMCs were washed three times in PBS before being processed.

4.3. Animal Husbandry

In vivo experiments were performed in female 7-week-old immunodeficient NOD.CB17/AlhnRj-Prkdcscid/Rj (NOD/SCID) mice characterized by T cell, B cell, and natural killer cell deficiency (Harlan, Guana, France). The mice were maintained under standardized environmental conditions in rodent micro-isolator cages (20±1° C. room temperature, 50±10% relative humidity, 12 hours light dark cycle). Mice received irradiated food and bedding and 0.22 μm-filtered drinking water. All experiments were performed according to the Swiss Animal Protection Law with previous authorization from the cantonal and federal veterinary authorities. In compliance with the Animal Protection Law, mice were euthanized when tumors induced by subcutaneous (s.c.) xenografts reached 2000 mm³.

4.4. Xenograft Experiments

A mix of tumor cells (target cells, T) and PBMCs (effector cells, E) were injected s.c. at an E:T ratio of 2:1 into the right flank area of NOD.CB17/AlhnRj-Prkdcscid/Rj (NOD/SCID) mice (n=5 per group per PBMC donor).

Protocols were prophylactic, the treatments were administered intravenously (i.v.) 3 hours post cell implantation.

CD3×EGFR_1 was administered i.v. once a week for 3 weeks.

The tumor size quantification was performed by caliper measurement. The tumor volumes were calculated using the following formula:

Tumor volume (mm3)=0.5×length×width2

Summary and Results

With reference to FIG. 9, the expression level of EGFR on A549 cells was determined by sABC. A549 tumor cells and PBMCs, obtained from healthy human donors, were injected s.c. into the right flank of female NOD/SCID mice. A total of 5 mice were grafted for each PBMC donor. CD3×EGFR_1 was administered i.v. once a week starting on day 0 for 3 weeks. Tumor growth was determined by external caliper measurements as described in Section 4.4. Control mice were treated with PBS. Statistical analysis performed: one-way analysis of variance (ANOVA) followed by Dunnett's post hoc for multiple comparisons. For each condition (control or CD3×EGFR_1), 2 PBMC donors were included (n=10 animals per condition).

In conclusion, CD3×EGFR_1 showed no efficacy in A549 tumors.

### EXAMPLE 5. COMPARISON OF CD3-EGFR-SF1 AND CD3-EGFR-SF3 EFFICACY IN A549 TUMORS XENOGRAFTED SUBCUTANEOUSLY

Material and Methods

5.1. Cell Line Culture Conditions

Cells were cultured in standard media in a humidified atmosphere of 5% CO₂ at 37° C. The cells were passaged 2 to 3 times per week to maintain them at optimal confluency and were routinely tested for mycoplasma contamination using the MycoAlert detection kit (LT07-318, Lonza). The cells consistently tested free of mycoplasma contamination.

5.2. Effector Cells: Human Peripheral Blood Mononuclear Cells (PBMC)

Peripheral blood mononuclear cells (PMBCs) were harvested from blood filters obtained from La Chaux-de-Fonds Transfusion Center. For blood filter processing, 40 ml of PBS supplemented with 1% Liquemine® (Drossapharm) was injected into the blood filter and the solution containing the PBMCs was collected in 50 ml blood separation tubes (Chemie Brunschwig, PAA535710) previously loaded with ficoll (GE Healthcare, 17-1440-03). The ficoll tubes were centrifuged for 20 min at 800 g at room temperature (RT) without the brake and the PBMC “buffy coat” ring was harvested and transferred into 50 ml falcon tubes containing 30 ml of PBS. The PBMCs were washed three times in PBS before being processed.

5.3. Animal Husbandry

In vivo experiments were performed with female 7-week-old immunodeficient NOD.CB17/AlhnRj-Prkdcscid/Rj (NOD/SCID) mice characterized by T cell, B cell, and natural killer cell deficiency (Harlan, Guana, France). The mice were maintained under standardized environmental conditions in rodent micro-isolator cages (20±1° C. room temperature, 50±10% relative humidity, 12 hours light dark cycle). Mice received irradiated food and bedding and 0.22 μm-filtered drinking water. All experiments were performed in accordance with Swiss Animal Protection Laws with previous authorization from the Swiss cantonal and federal veterinary authorities. In compliance with the Animal Protection Law, mice were euthanized when tumors induced by subcutaneous (s.c.) xenografts reached 2000 mm³.

5.4. Xenograft Experiments

A mix of tumor cells (target cells, T) and PBMCs (effector cells, E) were injected s.c. at an E:T ratio of 1:1 into the right flank area of NOD.CB17/AlhnRj-Prkdcscid/Rj (NOD/SCID) mice (n=5 per group per PBMC donor).

Protocols were prophylactic, the treatments were administered intravenously (i.v.) 3 hours post cell implantation.
CD3×EGFR-SF1 was administered i.v. once a week for 3 weeks.
CD3×EGFR-SF3 was administered i.v. once a week for 3 weeks.

The tumor size quantification was performed by caliper measurement. The tumor volumes were calculated using the following formula:

Tumor volume (mm3)=0.5×length×width2

5.5. Statistical Treatment

Data were analyzed using Graphpad Prism 5 software; the data are presented as the mean±SEM. Statistical analysis was performed by one-way analysis of variance (ANOVA) followed by Dunnett's post hoc for multiple comparisons. P<0.05 was considered as statistically significant.

Summary and Results

With reference to FIG. 10, CD3×EGFR-SF1 and CD3×EGFR-SF3 has the same efficacy in A549 tumors. The expression level of EGFR on A549 cells was determined by sABC. Equal numbers (10×10⁶) of A549 tumor cells and PBMCs obtained from healthy human donors were injected s.c. into the right flank of female NOD/SCID mice. A total of 5 mice were grafted for each PBMC donor. CD3×EGFR-SF1 and CD3×EGFR-SF3 were administered i.v. once a week starting on day 0 for 3 weeks. Tumor growth was determined by external caliper measurements as described in Section 5.4. Control mice were treated with PBS. Statistical analysis performed: one-way analysis of variance (ANOVA) followed by Dunnett's post hoc for multiple comparisons. For each condition (control, CD3×EGFR-SF1 or CD3×EGFR-SF3), 3 PBMC donor were included (n=15 animals per condition).

In conclusion CD3×EGFR-SF1 and CD3×EGFR-SF3 have the same efficacy in xenographed A549 tumors.

TABLE 1
Statistical analysis of FIG. 10. Statistical analysis
performed: one-way analysis of variance (ANOVA) followed
by Dunnett's post hoc for multiple comparisons.

| Dunnett's multiple | | | Adjusted |
|---|---|---|---|
| comparisons test | Significant | Summary | P Value |
| CD3 × EGFR-SF1 - 0.2 mg/kg | Yes | *** | <0.001 |
| vs. Control | | | |
| CD3 × EGFR-SF1 - 0.04 mg/kg | Yes | *** | <0.001 |
| vs. Control | | | |
| CD3 × EGFR-SF3 - 0.2 mg/kg | Yes | *** | <0.001 |
| vs. Control | | | |
| CD3 × EGFR-SF3 - 0.04 mg/kg | Yes | *** | <0.001 |
| vs. Control | | | |

### EXAMPLE 6: CD3×EGFR-SF3 DISPLAYS A BETTER IN VIVO ANTI-CANCER POTENCY THAN OTHER EGFR TARGETING THERAPIES (I)

Material and Methods

6.1. Cell Line Culture Conditions

Cells were cultured in standard media in a humidified atmosphere of 5% CO₂ at 37° C. The cells were passaged 2 to 3 times per week to maintain them at optimal confluency and were routinely tested for mycoplasma contamination using the MycoAlert detection kit (LT07-318, Lonza). The cells consistently tested negative for mycoplasma contamination.

6.2. Effector Cells: Human Peripheral Blood Mononuclear Cells (PBMC)

Peripheral blood mononuclear cells (PMBCs) were harvested from blood filters obtained from La Chaux-de-Fonds Transfusion Center. For blood filter processing, 40 ml of PBS supplemented with 1% Liquemine® (Drossapharm) was injected into the blood filter and the solution containing the PBMCs was collected in 50 ml blood separation tubes (Chemie Brunschwig, PAA535710) previously loaded with ficoll (GE Healthcare, 17-1440-03). The ficoll tubes were centrifuged for 20 min at 800 g at room temperature (RT) without the brake and the PBMC “buffy coat” ring was harvested and transferred into 50 ml falcon tubes containing 30 ml of PBS. The PBMCs were washed three times in PBS before being processed.

6.3. Animal Husbandry

In vivo experiments were performed in female 7-week-old immunodeficient NOD.CB17/AlhnRj-Prkdcscid/Rj (NOD/SCID) mice characterized by T cell, B cell, and natural killer cell deficiency (Harlan, Guana, France). The mice were maintained under standardized environmental conditions in rodent micro-isolator cages (20±1° C. room temperature, 50±10% relative humidity, 12 hours light dark cycle). Mice received irradiated food and bedding and 0.22 μm-filtered drinking water. All experiments were performed according to the Swiss Animal Protection Law with previous authorization from the cantonal and federal veterinary authorities. In compliance with the Animal Protection Law, mice were euthanized when tumors induced by subcutaneous (s.c.) xenografts reached 2000 mm3.

6.4. Xenograft Experiments

A mix of tumor cells (target cells, T) and PBMCs (effector cells, E) were injected s.c. at an E:T ratio of 1:1 into the right flank area of NOD.CB17/AlhnRj-Prkdcscid/Rj (NOD/SCID) mice (n=5 per group per PBMC donor).

Protocols were prophylactic, the treatments were administered intravenously (i.v.) 3 hours post cell implantation.
CD3×EGFR-SF3 was administered i.v. once a week for 3 weeks.
Vectibix was administered i.v. twice a week for 6 weeks.

The tumor size quantification was performed by caliper measurement. The tumor volumes were calculated using the following formula:

Tumor volume (mm3)=0.5×length×width2

6.5. Statistical Treatment

Data were analyzed using Graphpad Prism 5 software; the data are presented as the mean±SEM. Statistical analysis was performed by one-way analysis of variance (ANOVA) followed by Dunnett's post hoc for multiple comparisons. P<0.05 was considered as statistically significant.

Summary and Results

With reference to FIG. 11, the expression level of EGFR on SNU-216 cells was determined by sABC. Equal numbers (10×106) of SNU-216 tumor cells and PBMCs obtained from healthy human donors were injected s.c. into the right flank of female NOD/SCID mice. A total of 5 mice were grafted for each PBMC donor. CD3×EGFR-SF3 was administered i.v. once a week starting on day 0 for 3 weeks. Vectibix was administered i.v. twice a week starting on day 0 for 6 weeks. Tumor growth was determined by external caliper measurements as described in Section 1.4. The graphs show the mean tumor size (in mm3)±SEM. Control mice were treated with PBS. 1 PBMC donor was included (n=5 animals per condition). Name of the study: SNU_2

In conclusion CD3×EGFR-SF3 displays a better potency than Vectibix in SNU-216 tumors.

### EXAMPLE 7

CD3×EGFR-SF3 Displays a Better In Vivo Anti-Cancer Potency than Other EGFR Targeting Therapies (ii).

Material and Methods

7.1. Cell Line Culture Conditions

Cells were cultured in the media in a humidified atmosphere of 5% CO2 at 37° C. The cells were passaged 2 to 3 times per week to maintain them at optimal confluency and were routinely tested for mycoplasma contamination using the MycoAlert detection kit (LT07-318, Lonza). The cells consistently tested negative mycoplasma contamination free.

7.2. Effector Cells: Human Peripheral Blood Mononuclear Cells (PBMC)

Peripheral blood mononuclear cells (PMBCs) were harvested from blood filters obtained from La Chaux-de-Fonds Transfusion Center. For blood filter processing, 40 ml of PBS supplemented with 1% Liquemine® (Drossapharm) was injected into the blood filter and the solution containing the PBMCs was collected in 50 ml blood separation tubes (Chemie Brunschwig, PAA535710) previously loaded with ficoll (GE Healthcare, 17-1440-03). The ficoll tubes were centrifuged for 20 min at 800 g at room temperature (RT) without the brake and the PBMC “buffy coat” ring was harvested and transferred into 50 ml falcon tubes containing 30 ml of PBS. The PBMCs were washed three times in PBS before being processed.

7.3. Animal Husbandry

In vivo experiments were performed in female 7-week-old immunodeficient NOD.CB17/AlhnRj-Prkdcscid/Rj (NOD/SCID) mice characterized by T cell, B cell, and natural killer cell deficiency (Harlan, Guana, France). The mice were maintained under standardized environmental conditions in rodent micro-isolator cages (20±1° C. room temperature, 50±10% relative humidity, 12 hours light dark cycle). Mice received irradiated food and bedding and 0.22 μm-filtered drinking water. All experiments were performed according to the Swiss Animal Protection Law with previous authorization from the cantonal and federal veterinary authorities. In compliance with the Animal Protection Law, mice were euthanized when tumors induced by subcutaneous (s.c.) xenografts reached 2000 mm3.

7.4. Xenograft Experiments

A mix of tumor cells (target cells, T) and PBMCs (effector cells, E) were injected s.c. at an E:T ratio of 1:1 into the right flank area of NOD.CB17/AlhnRj-Prkdcscid/Rj (NOD/SCID) mice (n=5 per group per PBMC donor).

Protocols were prophylactic, the treatments were administered intravenously (i.v.) 3 hours post cell implantation.
CD3×EGFR-SF3 was administered i.v. once a week for 3 weeks.
Dexamethasone was administered i.p. three times a week for 3 weeks.

The tumor size quantification was performed by caliper measurement. The tumor volumes were calculated using the following formula:

Tumor volume (mm³)=0.5×length×width2

7.5. Statistical Treatment

Data were analyzed using Graphpad Prism 5 software; the data are presented as the mean±SEM. Statistical analysis was performed by one-way analysis of variance (ANOVA) followed by Dunnett's post hoc for multiple comparisons. P<0.05 was considered as statistically significant.

Summary and Results

With reference to FIG. 12. Dexamethasone impact on CD3×EGFR-SF3 anti-tumor activity in xenograft models. The expression level of EGFR on A549 cells was determined by sABC. Equal numbers (10×10⁶) of A549 tumor cells and PBMCs obtained from healthy human donors were injected s.c. into the right flank of female NOD/SCID mice. A total of 5 mice were grafted for each PBMC donor. CD3×EGFR-SF3 was administered i.v. once a week starting on day 0 for 3 weeks. Dexamethasone was administered i.p. three times a week starting on day 0 for 3 weeks. Tumor growth was determined by external caliper measurements as described in Section 7.4. Control mice were treated with PBS. Statistical analysis performed: one-way analysis of variance (ANOVA) followed by Dunnett's post hoc for multiple comparisons. For each condition (control, CD3×EGFR-SF3, Dexamethasone or the combo), 1 PBMC donor was included (n=5 animals per condition). (A) The graph shows the mean tumor size (in mm3)±SEM. (B) The graph shows the tumor growth per mouse at day 37. Name of the study: A549_10.

TABLE 2
Statistical analysis FIG. 12. Statistical analysis
performed: one-way analysis of variance (ANOVA) followed
by Dunnett's post hoc for multiple comparisons.

| Dunnett's multiple | | | Adjusted |
|---|---|---|---|
| comparisons test | Significant | Summary | P Value |
| Control vs. | Yes | **** | 0.0001 |
| CD3 × EGFR-SF3 - 0.05 mg/kg | | | |
| Control vs. | Yes | *** | 0.0002 |
| CD3 × EGFR-SF3 - 0.05 mg/kg + | | | |
| DEX - 0.5 mg/kg | | | |
| Control vs. | Yes | *** | 0.0005 |
| CD3 × EGFR -SF3 - 0.05 mg/kg + | | | |
| DEX - 5 mg/kg | | | |
| Control vs. DEX - 5 mg/kg | Yes | ** | 0.0066 |

In conclusion, administration of dexamethasone reduced the CD3×EGFR-SF3 anti-tumor activity in xenograft models of A549 cells.

### EXAMPLE 8: IN VIVO STABILITY OF CD3×EGFR-SF3 IN MICE SERUM

Material and Methods

8.1. Cell Line Culture Conditions

Cells were cultured in the media in a humidified atmosphere of 5% CO2 at 37° C. The cells were passaged 2 to 3 times per week to maintain them at optimal confluency and were routinely tested for mycoplasma contamination using the MycoAlert detection kit (LT07-318, Lonza). The cells consistently tested negative for mycoplasma contamination.

8.2. Effector Cells: Human Peripheral Blood Mononuclear Cells (PBMC)

Peripheral blood mononuclear cells (PMBCs) were harvested from blood filters obtained from La Chaux-de-Fonds Transfusion Center. For blood filter processing, 40 ml of PBS supplemented with 1% Liquemine® (Drossapharm) was injected into the blood filter and the solution containing the PBMCs was collected in 50 ml blood separation tubes (Chemie Brunschwig, PAA535710) previously loaded with ficoll (GE Healthcare, 17-1440-03). The ficoll tubes were centrifuged for 20 min at 800 g at room temperature (RT) without the brake and the PBMC “buffy coat” ring was harvested and transferred into 50 ml falcon tubes containing 30 ml of PBS. The PBMCs were washed three times in PBS before being processed.

8.3. Animal Husbandry

In vivo experiments were performed in female 7-week-old immunodeficient NOD.CB17/AlhnRj-Prkdcscid/Rj (NOD/SCID) mice characterized by T cell, B cell, and natural killer cell deficiency (Harlan, Guana, France). The mice were maintained under standardized environmental conditions in rodent micro-isolator cages (20±1° C. room temperature, 50±10% relative humidity, 12 hours light dark cycle). Mice received irradiated food and bedding and 0.22 μm-filtered drinking water. All experiments were performed according to the Swiss Animal Protection Law with previous authorization from the cantonal and federal veterinary authorities. In compliance with the Animal Protection Law, mice were euthanized when tumors induced by subcutaneous (s.c.) xenografts reached 2000 mm3.

8.4. Xenograft Experiments
In the experiment named: IVS_3 (simple ELISA binding—EGFR arm) injected with the batch P1027

15 NOD.CB17/AlhnRj-Prkdcscid/Rj (NOD/SCID) mice with no tumor xenografted were injected once with 2 mg/kg of CD3×EGFR-SF3 molecules in i.v.

Several time points of blood samples were performed after CD3×EGFR-SF3 injections: t=0 h (one week before injection), t=6 h, t=24 h, t=48 h, t=96 h, t=148 h. For each time point, 3 mice were bled.

Blood samples were centrifuged 10 min at 14'000 RPM, mice serums were collected and frozen at −20° C.
In the experiment named: IVS_3 bis (simple ELISA binding—CD3 arm & dual ELISA binding—CD3 and EGFR arm) injected with the batch P1069

Mice used for the IVS_3 study were re-used 3 weeks after the IVS_3 study. Detection of CD3×EGFR-SF3 before injection was performed to confirm that they were no more CD3×EGFR-SF3 left in the animal's blood. NOD.CB17/AlhnRj-Prkdcscid/Rj (NOD/SCID) mice with no tumor xenografted were injected once with 2 mg/kg of CD3×EGFR-SF3 molecules in i.v.

Several time points of blood samples were performed after CD3×EGFR-SF3 injections: t=0 h (one week before injection), t=6 h, t=24 h, t=48 h, t=96 h, t=148 h. For each time point, 3 mice were bled.

Blood samples were centrifuged 10 min at 14'000 RPM, mice serums were collected and frozen at −20° C.
In the experiment named: IVS_4 (simple ELISA binding—EGFR arm) injected with the batch P1027

A mix of tumor cells HT29 (target cells, T) and PBMCs (effector cells, E) were injected s.c. at an E:T ratio of 2:1 into the right flank area of 20 NOD.CB17/AlhnRj-Prkdcscid/Rj (NOD/SCID) mice (n=10 per group per PBMC donor).

The tumor size quantification was performed by caliper measurement. The tumor volumes were calculated using the following formula: Tumor volume (mm3)=0.5×length×width2

When tumor reached 150-200 mm3, CD3×EGFR-SF3 was administered i.v. once at 2 mg/kg.

Several time points of blood samples were performed after CD3×EGFR-SF3 injections: t=6 h, t=24 h, t=48 h, t=96 h, t=148 h. For each time point, 4 mice were bled (2 mice in control group and 2 mice in CD3×EGFR-SF3 treated group).

Blood samples were centrifuged 10 min at 14′000 RPM, mice serums were collected and frozen at −20° C.

8.5. Simple ELISA Binding

FIG. 13 shows the assay format.

Human EGFR-I-IV-C-His-tagged or Hs CD3 1-26 N-term peptide-Fc-tagged protein was diluted in 1×PBS to 2 μg/ml and 100 μl was added to each well of a 96 well plate and incubated overnight at 4° C.

The following day the supernatant was removed and the plate was blocked with 200 μl PBS 2% BSA/well for 1 hr at room temperature (RT). The supernatant was removed. CD3×EGFR-SF3 was diluted to a range of concentrations in PBS 2% BSA or in PBS 2% BSA spike with mouse serum at 1/100+1/200+1/400+1/800+1/1600+1/3200 and 100 μl of antibody dilution was added to each well according to the plate layout. For the samples (mouse treated serum—IVS3-IVS4) were diluted at 1/100+1/200+1/400+1/800+1/1600+1/3200 and 100 μl of serum dilution was added to each well according to the plate layout and incubated for 1 hr at RT. The plate was washed ×5 with PBS 0.01% Tween. One hundred μl goat anti-human Fc HRP (1/1000 in PBS 2% BSA) or 100 μl goat anti-human Fab HRP (1/1000 in PBS 2% BSA) was added to each well and the plate was incubated for 1 hr at RT. The plate was washed ×5 times with PBS 0.01% Tween and 100 μl of 3,3′,5,5′-tetramethylbenzidine (TMB) solution was added to each well. The reaction was stopped after 5 min by the addition of 100 μl H2SO4 2N/well. The absorbance was read at 450 nm.

Data were then plotted and analyzed using Prism (GraphPad) software. To obtain a STD curve: Used GraphPad Prism 5, Transform X values using X=Log(X), Analysis Nonlinear Regression, Equation: Sigmoidal Dose Response. To obtain a concentration in ug/ml of the antibody concentration into the samples: use the following way from the Transform X values using X=Log(X) use again the Analysis Nonlinear Regression, Equation: Sigmoidal Dose Response and Interpolated the unknown values with the STD curve.

8.6. Dual Binding ELISA on CD3 and EGFR

A dual binding enzyme-linked immunosorbent assay (ELISA) assay, quantifying the binding of CD3×EGFR-SF3 to its targets CD3 and EGFR has been developed to confirm CD3×EGFR-SF3 biological activity. FIG. 14 shows the assay format.

High binding 96-well flat-bottom plates were coated with anti panitumumab at 2 μg/ml and the plates were incubated overnight at 4° C. The plates were then blocked with PBS-2% BSA for 1 hour at RT. CD3×EGFR-SF3 was diluted to a range of concentrations in PBS 2% BSA or in PBS 2% BSA spike with mouse serum at 1/100+1/200+1/400+1/800+1/1600+1/3200 and 100 μl of antibody dilution was added to each well according to the plate layout. For the samples (mouse treated serum—IVS3-IVS4) were diluted at 1/100+1/200+1/400+1/800+1/1600+1/3200 and 100 μl of serum dilution was added to each well according to the plate layout and incubated for 1 hr at RT. The plates were then washed 5× with PBS 0.01% Tween. One hundred μl of anti Id SP34 biotinylated was added at 0.1 μg/ml and the plates were incubated for 1 hour at RT.

Streptavidin-HRP solution at 1/4000 dilution in PBS-2% BSA was added and the plates incubated 1 hour at RT. The plates were then washed 5× with PBS 0.01% Tween. Finally, 100 μl of SuperSignal West Femto Maximum Sensitivity Substrate solution was added to each well. Luminescence was measured (with Gain 100, Optic position Top, Emission Hole, Integration time 1 sec, Read Height 1.00 mm) with a Synergy HT2-Spectrophotometer.

Data were then plotted and analyzed using Prism (GraphPad) software. To obtain a STD curve: Used GraphPad Prism 5, Transform X values using X=Log(X), Analysis Nonlinear Regression, Equation: Sigmoidal Dose Response. To obtain a concentration in ug/ml of the antibody concentration into the samples: use the following way from the Transform X values using X=Log(X) use again the Analysis Nonlinear Regression, Equation: Sigmoidal Dose Response and Interpolated the unknown values with the STD curve.

Summary and Results

With reference to FIGS. 15, 16 and 17 detection of CD3×EGFR-SF3 in mice serum by a simple and dual binding ELISA was performed and CD3×EGFR-SF3 was detected up to one week after the injection.

### EXAMPLE 9. PHARMACOKINETIC PROFILE OF CD3×EGFR-SF3 IN SPRAGUE-DAWLEY RATS SERUM

The pharmacokinetics of CD3×EGFR-SF3 was evaluated in male Sprague-Dawley rats (n=4) following a single intravenous injection at a dose of 1 mg/kg body weight. The blood samples for pharmacokinetic (PK) assessment were collected at pre-specified time points of 0.25, 1, 6, 24, 48, 96, 168, 336, 530, 672, 840 and 1008 hours post dose over a period of 42 days (six weeks).

The concentrations of CD3×EGFR-SF3 in these serum samples were quantified using an ELISA method. In this method Human α-Panitumumab antibody was used as the capturing antibody and biotinylated anti-id biotin SP34 as the detecting antibody. LLOQ of the assay was 6.25 ng/ml in undiluted SD rat serum. The serum concentrations vs time profiles were subjected to non-compartmental analysis (NCA) using Phoneix WinNonlin® version 7.0 to estimate PK parameters.

Following intravenous bolus injection, the maximum concentration (Cmax) was observed at the initial time points of 0.25 hrs post dose except for animal #M2 at 1 hr. The serum concentrations were above LLOQ (6.25 ng/mL) for 35 days post injection in all animals except #M3. In animal #M3, the serum concentration was quantifiable only up to 672 hrs. The serum concentration was below LLOQ in all animals at 1008 hrs (day 42).

Intravenous pharmacokinetics profiles were comparable across all the four rats (FIG. 18). The serum concentration profile appeared to follow a bi-exponential disposition with an initial distribution phase followed by a longer terminal elimination phase. CD3×EGFR-SF3 showed slow clearance and limited volume of distribution. The terminal elimination half-life (t½) of CD3×EGFR-SF3 in Sprague-Dawley rats was estimated to be approximately 4 days.

TABLE 3
Mean pharmacokinetic parameters of CD3 ×
EGFR-SF3 in Sprague-Dawley rats serum.

| Mean PK | | Mean | |
|---|---|---|---|
| parameters | Unit | (% CV) | |
| Cₘₐₓ | (ug/mL) | 19 | (12.9) |
| AUC₀₋₁₀₀₈ | (hr*ug/mL) | 842.7 | (11) |
| AUC_{0-inf} | (hr*ug/mL) | 844.4 | (11) |
| *Tₘₐₓ | (hr) | 0.25 | (0.25-1) |
| t_{1/2} | (hr) | 98.0 | (8.2) |
| Vz | (mL/kg) | 168.5 | (10.1) |
| Vss | (mL/kg) | 119.6 | (11.4) |
| CL | (mL/hr/kg) | 1.195 | (11) |
| MRT_{INF} | (hr) | 100.11 | (3.5) |

*Median (Range)
Cmax: The peak plasma concentration of a drug after administration.
AUC: area under the curve, the integral of the concentration-time curve.
Tmax: Time to reach Cmax. T1/2, the time required for the concentration of the drug to reach half of its original value.
Vz: Volume of distribution during terminal phase after intravenous administration.
Vss: Apparent volume of distribution at equilibrium determined after intravenous administration.
CL: clearance, the volume of plasma cleared of the drug per unit time.
MRTINF: mean residence time infinity.

### EXAMPLE 10: FURTHER INVESTIGATIONS OF IN VITRO PHARMACOLOGY

Material and Methods

10.1 Simple ELISA Binding

High binding 96-well flat-bottom plates (Corning) were coated overnight at 4° C. with either human EGFR-I-IV-his or human CD3 1-26 N-term peptide (2 ug/ml in 0.01M PBS). Plates were blocked with PBS+2% BSA for 1 hour at room temperature (RT). Serial dilutions of CD3×EGFR-SF3 (starting at 10 ug/ml, 1/3 dilutions) and control antibodies (10 ug/ml) were prepared in PBS+2% BSA and 100 ul was transferred into the assay plate and incubated for 1 hour at RT. The plates were then washed 5× with PBS+0.01% Tween 20, and anti-human IgG (Fab) HRP (1/2000) was added for 1 hour at RT. The plates were washed 5× with PBS+0.01% Tween 20, 100 ul of TMB substrate solution (3,3′,5,5′-Tetramethylbenzidine) was added to each well and the reaction was stopped between 1 to 10 min by adding 100 ul of H₂SO₄ (2N). Optical density was measured at 450 nm with a Synergy HT2-Spectrophotometer.

10.2 Dual Binding ELISA on CD3 and EGFR

A dual binding enzyme-linked immunosorbent assay (ELISA) assay, quantifying the binding of CD3×EGFR-SF3 to its targets CD3 and EGFR has been developed to confirm CD3×EGFR-SF3 biological activity. For this, high binding 96-well flat-bottom plates (Corning) were coated overnight at 4° C. with human recombinant EGFR-Fc (2 ug/ml, 0.01 M PBS). Plates were washed 5× with PBS+0.01% Tween 20 and blocked with PBS+2% BSA for 1 hour at room temperature (RT). Serial dilutions of CD3×EGFR-SF3 (starting at 4 ug/ml, % dilution) and control antibodies were done in PBS+2% BSA, and 100 ul was transferred into the assay plate and incubated for 1 hour at RT. The plates were then washed 5× with PBS+0.01% Tween 20, and biotinylated anti-human CD3E (0.5 ug/ml) was added for 1 hour at RT prior to incubation for 1 h at RT with Streptavidin-HRP (1/1000). The plates were then washed 5× with PBS+0.01% Tween 20, 100 ul of TMB substrate solution (3,3′,5,5′-Tetramethylbenzidine) was added to each well and the reaction was stopped between 1 to 10 min by adding 100 ul of H₂SO₄ (2N). Optical density was measured at 450 nm with a Synergy HT2-Spectrophotometer.

10.3 Simple FACS Binding

FACS simple binding was performed using PBMCs (to assess binding to CD3) or NCI-H1703 squamous cancer cells (to assess binding to EGFR). Cells were resuspended at 10⁶ cells/ml in FACS buffer (1×PBS+10% Versene+2% FBS), and 100 ul were added to U-bottom 96-well plates which were then centrifuged at 350 g for 3 min. Serial dilutions of CD3×EGFR-SF3 (10 ug/ml, 1/3 dilution) and control antibodies were added to the cells and incubated 30 min at 4° C. The cells were washed in FACS buffer and stained with the following antibodies (ThermoFisher): anti-human CD4 PE-eF610 (1/100), anti-human CD8a APC (1/100) and anti-human IgG (Fc-gamma specific) PE (1/200) for 20 min 4° C. Cells were washed with FACS buffer and resuspended in FACS buffer containing Sytox green viability dye (1/200), for 20 min at 4° C. and acquired on a CytoFlex (Beckman Coulter).

10.4 Antibody-Dependent Cell-Mediated Cytotoxicity (ADCC) Assays

For effector cells, PBMCs were harvested from whole blood filters using ficoll gradient purification. Briefly, PBS containing Liquemin (Drossapharm) was injected into the filters, collected into 50 ml blood separation tubes (SepMate-50; Stemcell) loaded with ficoll, and centrifuged at 1200 g for 10 min. The PBMCS were harvested and washed three times with PBS before isolation of NK cells using the NK Cell Isolation Kit (eBiosciences) according to the manufacturer's protocol. Isolated NKs were resuspended at 10⁶ cells/mL and incubated overnight at 37° C. with IL-2 (100 U/ml Peprotech). For the target cells, HPB-ALL, A-431 and A549 were washed in ADDC media (RPMI+2% FCS+1% Glut+1% NEAA+1% NaPyr+1% P/S) and resuspended at 0.2×10⁶ cells/ml in CDC media. Serial dilutions of CD3×EGFR-SF3 (80 nM, 1/10 dilution) and control antibodies were added to the target cells (1:1 ratio) and incubated 15-20 min at 37° C. Spontaneous killing (lower baseline) was obtained using untreated target cells. Maximum killing (upper baseline) was obtained using heat-shocked cell (cells were frozen at −80° C., and thawed 3×). NK cells (50'000 cells) were then added (E:T ratio of 5:1) and the samples were incubated for 4.5 h at 37° C. For A549 and A-413, the samples were centrifuged 3 min at 350 g, the supernatant was harvested and analyzed for cytotoxicity (LDH release) using the CytoTox 96® Non-Radioactive Cytotoxicity Assay (Promega) according to the manufacturer's protocol. Briefly, the supernatants were incubated with LDH substrate solution for 20-60 min before stopping with 50 ul of stop solution and the plates were read at 490 nM with a Synergy HT2-Spectrophotometer. For the HPB-ALL cells, the cells were resuspended in 1×PBS+10% Versene+2% FBS containing 7-AAD (1/100) for analysis on a CytoFlex (Beckman Coulter).

10.5 Complement-Dependent Cytotoxicity (CDC) Assays

Target cells (A549 or HPB-ALL) were washed in CDC media (RPMI+2% FCS+1% Glut+1% NEAA+1% NaPyr+1% P/S) and resuspended at 10⁶ cells/mL in CDC media. Serial dilutions of CD3×EGFR-SF3 (100 nM, 1/5 dilution) and control antibodies were added to the target cells (1:1 ratio) and incubated 15 min at 37° C., before adding 15% of baby rabbit complement. Spontaneous killing (lower baseline) was obtained using untreated target cells. Maximum killing (upper baseline) was obtained by heat-shocked cell (cells were frozen at −80° C., and thawed 3×). Samples were incubated for 4.5 h at 37° C. and centrifuged 3 min at 350 g. For the A549 cells, the supernatant was harvested and analyzed for cytotoxicity (LDH release) using the CytoTox 96® Non-Radioactive Cytotoxicity Assay (Promega) according to the manufacturer's protocol. Briefly, the supernatants were incubated with LDH substrate solution for 20-60 min before stopping with 50 ul of stop solution and the plates were read at 490 nM with a Synergy HT2-Spectrophotometer. For the HPB-ALL cells, the cells were resuspended in 1×PBS+10% Versene+2% FBS containing 7-AAD (1/100) for acquisition on a CytoFlex (Beckman Coulter). Data were analyzed using FlowJo (BD).

10.6 Normal Density PBMC Assay

PBMCs were harvested from whole blood filters using ficoll gradient purification. Briefly, PBS containing Liquemin (Drossapharm) was injected into the filters, collected into 50 ml blood separation tubes (SepMate-50; Stemcell) loaded with ficoll, and centrifuged at 1200 g for 10 min. The PBMCS were harvested, washed three times with PBS, resuspended at 10⁶ cells/ml, seeded in 96-well plates and incubated for 24 h or 48 h at 37° C. in the presence of serial dilutions of CD3×EGFR-SF3 (10 ug/ml, 1/3 dilution) and control antibodies.

Activation markers were assessed at 24 h and 48 h by flow cytometry. The cells were stained for 20 min at 4° C. with the following antibodies (ThermoFischer): anti-human CD4 PE-eF610, CD8 AF700, CD25 PE and CD69 PeCy7, then washed 1×PBS+10% Versene+2% FBS, centrifuged 3 min at 350 g and resuspended in 1×PBS+10% Versene+2% FBS containing Sytox green viability dye (1/2000), for 20 min at 4° C. and acquired on a CytoFlex (Beckman Coulter). Data were analyzed using FlowJo (BD).

For cytokine release at 24 h and 48 h, plates were centrifuged at 350 g for 5 min, supernatants were harvested and cytokines were quantified by Luminex according to the manufacturer's protocol.

To assess the proliferation, 3H-thymidine (0.5 uCu/well) was added after 30 h of incubation and harvested at the 48 h time-point user filtermate filters to which scintillation fluid is added before reading the counts per million (cpm) using a beta scintillation counter.

10.7 Non-Specific T Cell Activation in a High Density PBMC Assay

PBMCs were harvested from whole blood filters using ficoll gradient purification. Briefly, PBS containing Liquemin (Drossapharm) was injected into the filters, collected into 50 ml blood separation tubes (SepMate-50; Stemcell) loaded with ficoll, and centrifuged at 1200 g for 10 min. The PBMCS were harvested and washed three times with PBS and seeded in 24-well plates at 10⁷ cells/ml for incubation at 37° C. for 48 h. The cells were then centrifuged 5 min at 350 g, resuspended at a normal density of 10⁶ cells/ml in 96-well plates and incubated for 24 h at 37° C. in the presence of serial dilutions of CD3×EGFR-SF3 (10 ug/ml, 1/3 dilution) and control antibodies. To measure the cytokines released, plates were centrifuged at 350 g for 5 min, supernatants were harvested and cytokines were quantified by Luminex according to the manufacturer's protocol.

10.8 Whole Blood Assay (WBA)

Fresh whole blood was harvested from healthy volunteers and 0.5 ml/well were seeded in a 48-well plate. The blood was incubated for 24 h at 37° C. in the presence of serial dilutions of CD3×EGFR-SF3 (10 ug/ml, 1/10 dilution) and control antibodies. The samples were centrifuged for 5 min at 3000 g and supernatants were harvested, diluted ½ and cytokines released were quantified by Luminex according to the manufacturer's protocol.

10.9 Redirected Lysis Assay (RDL)

A range of cell lines were obtained from ATCC for use as target cells and passaged 2-3×/week with trypsin to maintain them at optimal confluency in the media recommended by the supplier. The cells were routinely tested for mycoplasma contamination using the Mycoalert Detection Kit (LT07-318, Lonza) and were consistently negative. Prior to each assay, the cells were assessed for specific Antibody Binding Capacity (sABC; QIFIKIT®) to verify the surface EGFR expression.

PBMCs (effector cells) were harvested from whole blood filters using ficoll gradient purification. Briefly, PBS containing Liquemin (Drossapharm) was injected into the filters, collected into 50 ml blood separation tubes (SepMate-50; Stemcell) loaded with ficoll, and centrifuged at 1200 g for 10 min. The PBMCS were harvested, washed three times with PBS and resuspended at 2×10⁶ cells/ml.

For the redirected lysis, target cells (T; 1×10⁴ cells/well) and effector cells (E; 1×10⁵ cells/well) (E:T ratio 10:1) were plated in 96-well flat bottom plates incubated for 48 h at 37° C. in the presence of serial dilutions of CD3×EGFR-SF3 (10 nM, 1/10 dilution) and control antibodies. The viability of the target cells was assessed at 48 h by MTS assay using the CellTiter 96® AQᵤₑₒᵤₛ One solution cell proliferation assay (Promega) according to the manufacturer's protocol. Briefly, the plates were washed 3 times and then the MTS solution was added into the wells. Plates were read at 490 nm on a Synergy HT2-Spectrophotometer. The plates were considered valid when a sufficient difference between maximum killing (target only that were killed using a Lysis solution) and spontaneous killing (wells with target only) was observed.

10.10 Data and Statistical Analysis

Dose response analysis: Data were plotted and analyzed using Prism (GraphPad). Data were first transformed using X=Log(X). Using transformed data a 4 parameters logistic regression (4PL) fitting was applied resulting in a sigmoidal dose-response curve (Hillslope fixed to 1). ECF values (F=20, 50 and 80) corresponding to the percentage F of the maximum efficacy of the tested sample were obtained according to the curve fitting.

Flow cytometry data: Data were analyzed using FlowJo (BD) and either mean fluorescence intensity (MFI), percentage of specific cells population or events by ul were extracted. Data were then processed for each experiment.

Luminex Data: Luminex data were analyzed using ProcartaPlex Analyst (eBioscience). Cytokines concentration were normalized to the upper and lower limit of quantification.

Percentage of Specific ADCC Killing Formula:

$$\% Specific {Killing}_{S a m p l e} = \frac{Sample - RS}{R M - R S} \times 100$$

Where sample corresponds to the killing measured in a sample, RS corresponds to the spontaneous killing and RM corresponds to the maximum killing. Percentages of specific killing were then analyzed using the dose response analysis method.

Percentage of Specific CDC Killing Formula:

% Specific CDC_{Sample}=% Specific Killing_{Sample}−% Specific killing_{No Antibody}

Where % Specific Killing_{Sample} corresponds to the specific killing measured for a sample, % Specific killing_{No Antibody} corresponds to the specific killing for an untreated target. Percentages of specific killing were then analyzed using the dose response analysis method.

Percentage of Specific Killing Formula in a RDL:

$$\% Specific {Killing}_{S a m p l e} = \frac{Abs 490 nm ( Spontaneous Killing ) - Abs 490 nm ( Sample )}{Abs 490 nm ( Spontaneous Killing ) - Abs 490 nm ( Maximum killing )} \times 100$$

Where Abs490 nm (Sample) correspond to the OD obtain for a sample, Abs490 nm (Spontaneous Killing) correspond to the OD obtain for the mean of target only wells and Abs490 nm (Maximum Killing) corresponds to the mean OD obtained for the lysed target cells only. Percentages obtained this way were further analyzed using the dose response analysis method described above.

Donor exclusion: Donor exclusion was performed using JMP software.

RDL donor exclusion: Donors were excluded when the fitting of the dose response curve had an R²<0.7, or when the no mAb samples had a specific killing higher than 40%.

Safety experiment donor exclusion: Readout data (Activation percentage, proliferation or cytokines concentration) were fitted against the treatment for each donor separately. A Dunnett's comparison test was then performed using the no mAb condition as the control. Donors were excluded when there was no statistical differences between the no mAb condition and at least one of the positive controls for that donor.

Statistical analysis: Statistical comparisons were performed using JMP. Fit Least Square nested models were performed to compare the effect of the treatment, the concentration of the treatment and the batch of the treatment. After the model fitting, a Dunnett's comparison was performed using the no mAb condition and the IgG Isotype condition as controls. These comparisons were performed after donor exclusion and for each time point (if applicable) separately.

Summary and Results

To confirm CD3×EGFR-SF3 biological activity and to quantify the binding of CD3×EGFR-SF3 to its targets CD3 and EGFR, ELISA assays have been performed.

In particular, a dose response of CD3×EGFR-SF3 and control antibodies were incubated on coated human CD3-Fc or human EGFR his-tagged then detected with either an anti-human IgG Fab coupled with HRP (single EGFR or CD3; FIGS. 19 A and B, respectively) or huCD3-biotin followed by HRP-coupled streptavidin (dual binding; FIG. 19C). Table 4 represents the EC20, 50 and 80 values extracted from the sigmoidal dose-response binding curves of three independent replications (FIG. 19).

TABLE 4
EC values of CD3 × EGFR-SF3 binding ELISA.
The values represent the mean ± SEM.

| | EC₂₀ | EC₅₀ | EC₈₀ |
|---|---|---|---|
| ELISA Binding | (ug/ml) | (ug/ml) | (ug/ml) |
| Single EGFR (A) | 0.0132 ± 0.004 | 0.053 ± 0.016 | 0.212 ± 0.063 |
| Single CD3 (B) | 0.023 ± 0.001 | 0.091 ± 0.005 | 0.363 ± 0.022 |
| Dual Binding (C) | 0.043 ± 0.01 | 0.171 ± 0.04 | 0.685 ± 0.18 |

To further assess CD3×EGFR-SF3 binding to CD3 and to EGFR, FACS simple binding was performed using PBMCs or NCI-H1703 squamous cancer cells, respectively. In particular a dose response of CD3×EGFR-SF3 and control antibodies were incubated on either PBMCS (FIG. 20 A-C) or the squamous cancer cell line NCI-H1703 (FIG. 20D) and detected with a PE-labelled anti-human IgG (Fc-γ). For the PBMCs, the cells were also labelled with anti-CD4 or anti-CD8 antibodies. Table 5 represents the EC20, 50 and 80 values extracted from the non-linear sigmoidal regression binding curves of three independent replications (FIG. 20).

TABLE 5
EC values of CD3 × EGFR-SF3 binding ELISA.
The values represent the mean ± SEM.

| FACS | | EC₂₀ | EC₅₀ | EC₈₀ |
|---|---|---|---|---|
| binding | / | (ug/ml) | (ug/ml) | (ug/ml) |
| CD3 | T cells (A) | 1.48 ± 0.32 | 5.93 ± 1.29 | 23.74 ± 5.162 |
| | CD4+ | 1.51 ± 0.29 | 6.05 ± 1.16 | 24.22 ± 4.67 |
| | T cells (B) | | | |
| | CD8+ | 0.976 ± 0.43 | 3.91 ± 1.75 | 15.617 ± 6.99 |
| | T cells (C) | | | |
| EGFR | NCI-H1703 | 0.046 ± 0.01 | 0.182 ± 0.05 | 0.730 ± 0.2 |
| | (D) | | | |

To assess the ability of CD3×EGFR-SF3 to induce the redirected lysis of various EGFR-expressing human cancer cell lines, target cancer cells (T) and effector cells (E; PBMCs) were incubated at an E:T ratio of 1:10 in the presence of a dose response of CD3×EGFR-SF3 or control antibodies. The redirected lysis of the cancer cells was determined by a cytotoxic assay (MTS). The EC₅₀ values were extracted from the sigmoidal dose-response curves of specific killing (FIG. 21). Cell lines redirected lysis was statistically different (one-way ANOVA; F=5, 6; p<0.0001). In conclusion, CD3×EGFR-SF3 induces the redirected lysis of all of the EGFR-expressing human cancer cell lines tested.

Antibody-dependent cell-mediated cytotoxicity (ADCC) of CD3×EGFR-SF3 was evaluated in the EGFR+ carcinoma cell lines A-431 and A549 (FIG. 22 A) as well as in CD3+ HPB-ALL cells (FIG. 22 B) and represented by the EC₅₀ values that were extracted from the sigmoidal dose-response curves of specific killing. Treatment with CD3×EGFR-SF3 reduced the ADCC as compared to Erbitux in both A-431 and A549 (FIG. 22A; Least Square model, F=29, p<0.0001) and as compared to human anti-SP34 antibody in HPB-ALL cells (FIG. 22B; T test, t=3, p<0.05). In conclusion, CD3×EGFR-SF3 does not induce ADCC in the EGFR- or CD3-expressing cell lines tested.

Specific complement-dependent cytotoxicity (CDC) was evaluated in the EGFR+ carcinoma cells A549 (FIG. 23A) as well as in CD3+ HPB-ALL cells (FIG. 23B) and the EC₅₀ values were extracted from the sigmoidal dose-response curves of specific CDC. For both A549 and HPB-ALL cells, CD3×EGFR-SF3 does not induce any specific complement-dependent cytotoxicity.

To evaluate the effects of CD3×EGFR-SF3 on non-specific cellular proliferation, PBMCs from healthy donors (n=19) were incubated for 48 h in presence of increasing doses of CD3×EGFR-SF3 or controls. Proliferation was assessed by measuring the incorporation of ³H-thymidine at 48 h (FIG. 24). Statistical analysis of the proliferation was done using a Fit Least Square model followed by a Dunnett's comparison to compare to means either against the no mAb control (FIG. 25A) or against the isotype control (FIG. 25B).

In comparison to the no mAb condition, CD3×EGFR-SF3 induced a slight proliferation at high concentration of the batches of CD3×EGFR-SF3 AE042 and P1069 which is not observed in the most recent bulk drug substance the TRS batch. When compared to the isotype control, CD3×EGFR-SF3 only induced statistically significant proliferation in the AE042 batch at the highest concentration (5 ug/ml). The other batches of CD3×EGFR-SF3 in either aqueous or wet coated form did not induce any significant increase in proliferation as compared to the isotype control. In conclusion, the TRS batch of CD3×EGFR-SF3 does not induce any proliferation in a PBMC assay.

To evaluate whether CD3×EGFR may induce a non-specific activation of CD4+ T cells, PBMCs from healthy donors (n=23) were incubated for 24 h or 48 h in presence of increasing doses of CD3×EGFR-SF3 or controls. Activation of CD4+ T cell was measured as the expression of the T cell activation marker CD69 by flow cytometry (FIG. 26). Statistical analysis of the CD4+ T cell activation was performed for each time-point using a Fit Least Square model followed by a Dunnett's comparison to compare to means either against the no mAb control (FIGS. 27A and B) or against the isotype control (FIGS. 28 A and B).

When compared to the no mAb condition (i.e. the most stringent comparison), the coated and aqueous TRS batch of CD3×EGFR-SF3 at high concentration (1-10 ug/ml), and aqueous only AE042 batch of CD3×EGFR-SF3 at 5 ug/ml induced a non-specific activation of CD4+ T cells at 24 h and 48 h, and the aqueous but not coated CD3×EGFR-SF3 batch P1069 (at concentrations starting at 0.005 ug/ml) induced CD4+ T cells activation at 48 h. When compared to the isotype control, none of the batches of CD3×EGFR-SF3 tested induced a non-specific CD4+ T cell activation at 24 h, and at 48 h only the highest concentration (5 ug/ml) of the aqueous form of the batches AE042 and P1069 but not the TRS batch of CD3×EGFR-SF3 induced activation of CD4+ T cells. In conclusion, as compared to an isotype antibody the TRS batch of CD3×EGFR-SF3 does not induce statistically significant CD4+ T cell activation in a non-specific PBMC.

To evaluate whether CD3×EGFR may induce a non-specific activation of CD8+ T cells, PBMCs from healthy donors (n=23) were incubated for 24 h or 48 h in presence of increasing doses of CD3×EGFR-SF3 or controls. Activation of CD8+ T cell was measured as the expression of the activation marker CD69 by flow cytometry (FIG. 29). Statistical analysis of the CD8+ T cell activation was performed for each time-point using a Fit Least Square model followed by a Dunnett's comparison to compare to means either against the no mAb control (FIGS. 30A and B) or against the isotype control (FIGS. 31 A and B).

When compared to the no mAb condition (i.e. the most stringent comparison), the different batches of CD×EGFR-SF3 either aqueous or coated at high concentration (1-10 ug/ml) induced CD8+ T cell activation at 24 h and 48 h in a PBMC assay. When compared to the isotype control, at 24 h only the coated TRS batch of CD3×EGFR-SF3 at 1 ug/mL, but not 10 ug/ml induced CD8+ T cell activation, and at 48 h, only the highest concentrations of the different batches induced CD8+ T cell activation. In conclusion, as compared to an isotype antibody CD3×EGFR-SF3 does not induce statistically significant CD8+ T cell activation in a non-specific PBMC assay at low doses.

To predict any potential cytokine release in a clinical setting, PBMCs are routinely used in pre-clinical testing for cytokine release assays (Stebbings et al. J Immunol 179:3325-3331 (2007)). To evaluate whether CD3×EGFR-SF3 may induce a non-specific T cell cytokine response, PBMCs from healthy donors (n=23) were incubated for 24 h in presence of increasing doses of CD3×EGFR-SF3 or controls, and the levels of IL-2, IL-6, TNF-α, and IFN-γ released were measured by Luminex in the supernatant (FIG. 32). Statistical analysis of the cytokines released was performed for each time-point using a Fit Least Square model followed by a Dunnett's comparison to compare to means either against the no mAb control (FIG. 33A-D) or against the isotype control (FIG. 34A-D).

When compared to the no mAb condition (i.e. the most stringent comparison), none of the batches of CD3×EGFR-SF3 induced the release of IL-2, only the aqueous AE042 batch of CD3×EGFR-SF3 induced the release of IL-6, and only the higher doses (0.5 and 5 ug/ml) of coated AE042 and P1069 batches of CD3×EGFR-SF3 induced IFN-γ and TNF-α release in a non-specific T cell assay. When compared to the isotype control, CD3×EGFR-SF3 did not induce the release of either IL-2 or IFN-γ at 24 h, IL-6 was only induced in presence of aqueous AE042 batch of CD3×EGFR-SF3 at the highest concentrations, and TNF-α only with the coated P1069 batch of CD3×EGFR-SF3 at the highest concentrations. In summary, CD3×EGFR-SF3 only induces the release of IL-6 and TNF-α, but not IL-2 or IFN-γ at the highest concentrations tested, in a batch-dependent manner following 24 h incubation with PBMCs.

PBMCs from healthy donors (n=23) were incubated for 48 h in presence of increasing doses of CD3×EGFR-SF3 or controls, and the levels of IL-2, IL-6, TNF-α, and IFN-γ released were measured by Luminex in the supernatant (FIG. 35). Statistical analysis of the cytokines released was performed for each time-point using a Fit Least Square model followed by a Dunnett's comparison to compare to means either against the no mAb control (FIG. 36A-D) or against the isotype control (FIG. 37A-D).

When compared to the no mAb condition (i.e. the most stringent comparison), none of the batches of CD3×EGFR-SF3 induced the release of IL-2 or IFN-γ, only coated CD3×EGFR-SF3 at high concentrations induced the release of TNF-α, and IL-6 release was much more variable depending on the batch and concentration of CD3×EGFR-SF3. When compared to the isotype control, CD3×EGFR-SF3 did not induce any release of either IL-2 or IFN-γ, coated CD3×EGFR-SF3 induced the release of TNF-α at high concentrations only and IL-6 release was induced with the aqueous but not coated AE042 batch of CD3×EGFR-SF3 (0.05, 0.5, and 5 ug/ml), and in coated TRS at 0.01 and 0.1 ug/ml but not at any higher concentrations. In summary, CD3×EGFR-SF3 only induces the release of IL-6 and TNF-α, but not IL-2 and IFN-γ at high concentrations, in a batch-dependent manner following 48 h incubation with PBMCs.

High density pre-culture of PBMCs followed by incubation with soluble mAbs is used as a cytokine release assay method to evaluate the pre-clinical safety of mAbs (Römer et al. Blood 118:6772-6782 (2011)). PBMCs from healthy donors (n=16) were pre-incubated for 48 h at high density (10⁷ cells/ml). The cells were then plated at a normal density (10⁶ cells/ml), and cultured for 24 h in presence of increasing doses of aqueous CD3×EGFR-SF3 or controls, and the levels of IL-2, IL-6, TNF-α, and IFN-γ released were measured by Luminex in the supernatant (FIG. 38). Statistical analysis of the cytokines released was performed using a Fit Least Square model followed by a Dunnett's comparison to compare to means either against the no mAb control (FIG. 39A-D) or against the isotype control (FIG. 40A-D). CD3×EGFR-SF3 did not induce any significant increase in the levels of IL-2, IL-6, TNF-α, or IFN-γ as compared to either the untreated (no mAb) condition or the isotype control in a high density PBMC assay.

Whole blood assays are widely used as a risk-assessment method to identify to potential release of cytokines in a clinical setting following the infusion with monoclonal antibody therapies (Vessillier et al. Immunol Methods 424:43-52 (2015)). To assess whether CD3×EGFR-SF3 may induce cytokine production, whole blood from healthy volunteers (n=16) was cultured for 24 h in presence of increasing doses of CD3×EGFR-SF3 or controls and the plasma levels of IL-2, IL-6, TNF-α, and IFN-γ were measured by Luminex in the supernatant (FIG. 41). Statistical analysis of the cytokines released was done using a Fit Least Square model followed by a Dunnett's comparison to compare to means either against the no mAb control (FIG. 42A-D) or against the isotype control (FIG. 43A-D). CD3×EGFR-SF3 did not induce any significant increase in the levels of IL-2, IL-6, TNF-α, or IFN-γ as compared to either the untreated (no mAb) condition or the isotype control in a whole blood assay.

### EXAMPLE 11. FURTHER IN VIVO CHARACTERIZATION OF CD3×EGFR_SF3 IN NOD SCID XENOGRAFTED MOUSE MODEL

Material and Methods

11.1 Cell Line Culture Conditions

A549 cells were cultured in the media in a humidified atmosphere of 5% CO₂ at 37° C. The cells were passaged 2 to 3 times per week to maintain them at optimal confluency and were routinely tested for mycoplasma contamination using the MycoAlert detection kit. The cells consistently tested negative.

11.2 Effector Cells: Human Peripheral Blood Mononuclear Cells (PBMC)

Peripheral blood mononuclear cells (PMBCs) were harvested from blood filters obtained from La Chaux-de-Fonds Transfusion Center. For blood filter processing, 40 ml of PBS supplemented with 1% Liquemine® (Drossapharm) was injected into the blood filter and the solution containing the PBMCs was collected in 50 ml blood separation tubes (Chemie Brunschwig) previously loaded with ficoll (GE Healthcare). The ficoll tubes were centrifuged for 20 min at 800 g at room temperature (RT) without the brake and the PBMC “buffy coat” ring was harvested and transferred into 50 ml falcon tubes containing 30 ml of PBS. The PBMCs were washed three times in PBS before being processed.

11.3 Animal Husbandry

In vivo experiments were performed in female 6-7-week-old immunodeficient NOD.CB17/AlhnRj-Prkdcscid/Rj (NOD/SCID) mice characterized by T cell, B cell, and natural killer cell deficiency (Envigo). The mice were maintained under standardized environmental conditions in rodent micro-isolator cages (20±1° C. room temperature, 50±10% relative humidity, 12 hours light dark cycle). Mice received irradiated food and bedding and 0.22 μm-filtered drinking water. All experiments were performed according to the Swiss Animal Protection Law with previous authorization from the cantonal and federal veterinary authorities.

11.4 Xenograft Experiments

A mix of tumor cells (target cells, T) and PBMCs (effector cells, E) were injected s.c. at an E:T ratio of 2:1 into the right flank area of NOD.CB17/AlhnRj-Prkdcscid/Rj (NOD/SCID) mice (n=4 to 5 per group per PBMC donor). Protocol was therapeutic, the treatment was administered intravenously (i.v.) day 2 post cell implantation. CD3×EGFR-SF3 (P1069) was administered i.v. once a week for 3 weeks at 2 mg/kg. The tumor size quantification was performed by caliper measurement. The tumor volumes were calculated using the following formula: Tumor volume (mm3)=0.5×length×width2.

11.5 Statistical Analysis

Data were analyzed using Graphpad Prism 5 software; the data are presented as the mean±SEM. Statistical analysis was performed by a Mann-Whitney test. P<0.05 was considered as statistically significant.

Summary and Results

TABLE 6
Statistical analysis of FIG. 45 (Mann Whitney test).

| Mann Whitney test | |
|---|---|
| P value | 0.0184 |
| Exact or approximate P value? | Exact |
| P value summary | * |
| Significantly different (P < 0.05)? | Yes |

At day 41, in CD3×EGFR-SF3 treated group, the mean of the tumor volume was 488 mm3 compared to 1059 mm3 in the control group (see FIGS. 44 and 45). CD3×EGFR-SF3 induced a significant A549 tumor grow reduction.

1. A CD3×EGFR bispecific antibody which binds to CD3ε and EGFR epitopes, wherein the antibody comprises at least one Fab and at least one scFv binding portion.

2. (canceled)

3. The CD3×EGFR bispecific antibody of claim 1, wherein said at least one Fab and at least one scFv binding portion are concatenated to each other.

4. The CD3×EGFR bispecific antibody of claim 1, selected from the group consisting of CD3×EGFR_SF1 (SEQ ID NOs: 4, 5 and 6), CD3×EGFR_SF3 (SEQ ID NOs: 7, 2 and 8), CD3×EGFR_SF4 (SEQ ID NOs: 4, 5 and 9), CD3×EGFR_SD1 (SEQ ID NOs: 1, 2 and 10), and CD3×EGFR_SD2 (SEQ ID NOs: 11, 10 and 2).

5. A method of treating cancer in a subject in need thereof, comprising administering an effective amount of the CD3×EGFR bispecific antibody of claim 4 to treat the cancer.

6. The method of claim 5, wherein said cancer is an EGFR expressing cancer.

7. The method of claim 6, wherein said EGFR expressing cancer further comprises one or more KRAS mutation or B-Raf mutation.

8. An antibody or fragment thereof that binds to domain 4 of human EGFR, wherein the antibody or fragment thereof comprises a heavy chain variable domain and a light chain variable domain sequence selected from the group consisting of SEQ ID NOs: 23 and 24, SEQ ID NOs: 25 and 26, SEQ ID NOs: 31 and 33, SEQ ID NOs: 32 and 34, SEQ ID NOs: 36 and 38, and SEQ ID NOs: 37 and 39.

9. A method of treating a disease characterized or exacerbated by overexpression of EGFR in a subject in need thereof, comprising administering an effective amount of the CD3×EGFR bispecific antibody of claim 4 to treat the disease characterized or exacerbated by overexpression of EGFR.

10. The method of claim 9, wherein the disease is an EGFR expressing cancer.

11. The method of claim 10, wherein said EGFR expressing cancer further comprises one or more KRAS mutation or B-Raf mutation.

12. An in vitro method for the production of the CD3×EGFR bispecific antibody of claim 1, comprising the following steps:
(i)(a) preparing a DNA vector encoding an epitope binding portion of a first polypeptide and a DNA vector encoding an epitope binding portion of a second polypeptide, wherein the epitope binding portion of the first polypeptide is a Fab and the epitope binding portion of the second polypeptide is a scFv or wherein the epitope binding portion of the first polypeptide is a scFv and the epitope binding portion of the second polypeptide is a Fab; or
(i)(b) preparing one DNA vector encoding the Fab of the first polypeptide and the scFv of the second polypeptide or the scFv of the first polypeptide and the Fab of the second polypeptide; and wherein said DNA vectors are suitable for transient or stable expression in a mammalian host cell;
(ii) transfecting or co-transfecting the DNA vector(s) from (i) in a mammalian host cell line;
(iii) culturing the transfected cell line or stably selected clone therefrom and harvesting the cell culture supernatant;
(iv) contacting the cell culture supernatant on a Protein A affinity chromatography resin; and
(v) eluting and collecting the CD3×EGFR bispecific antibody from the culture.


SEQUENCE LISTING (52 sequences)

SEQ ID NO: 1 - protein, 449 aa, Artificial Sequence
  Synthetic Sequence
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 2 - protein, 214 aa, Artificial Sequence
  Synthetic Sequence
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 482 aa, Artificial Sequence
  Synthetic Sequence
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGK

SEQ ID NO: 4 - protein, 701 aa, Artificial Sequence
  Synthetic Sequence
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSEVQLVESGGGLVQPGGSLRLSCAASGFTFNTYAMNWVRQAPGKGLEWVARIRSKYNNYATYYADSVKSRFTISRDDSKNTLYLQMSSLRAEDTAVYYCVRHGNFGNSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 5 - protein, 217 aa, Artificial Sequence
  Synthetic Sequence
EIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 227 aa, Artificial Sequence
  Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGK

SEQ ID NO: 7 - protein, 703 aa, Artificial Sequence
  Synthetic Sequence
EVQLVESGGGLVQPGGSLRLSCAASGFTFNTYAMNWVRQAPGKGLEWVARIRSKYNNYATYYADSVKSRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKGGGGTQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 8 - protein, 226 aa, Artificial Sequence
  Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESDGSFALSSRLRVDKSRWQQGNVFSCSVMHEALHNHYTQKSLSLSPG

SEQ ID NO: 9 - protein, 473 aa, Artificial Sequence
  Synthetic Sequence
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGK

SEQ ID NO: 10 - protein, 709 aa, Artificial Sequence
  Synthetic Sequence
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGK

SEQ ID NO: 11 - protein, 227 aa, Artificial Sequence
  Synthetic Sequence
DKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 12 - protein, 704 aa, Artificial Sequence
  Synthetic Sequence
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGKGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIK

SEQ ID NO: 13 - protein, 5 aa, Artificial Sequence
  Synthetic Sequence
GGGGS

SEQ ID NO: 14 - protein, 5 aa, Artificial Sequence
  Synthetic Sequence
GGGGT

SEQ ID NO: 15 - protein, 624 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTSAHHHHHHHH

SEQ ID NO: 16 - protein, 144 aa, Artificial Sequence
  Synthetic Sequence
VCHALCSPEGCAGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTSAHHHHHHHH

SEQ ID NO: 17 - protein, 93 aa, Artificial Sequence
  Synthetic Sequence
IQCHPECLPQAMNITCTGRGPDNCIQCAHYIDGPHCVKTCPAGVMGENNTLVWKYADAGHVCHLCHPNCTYGCTGPGLEGCPTSAHHHHHHHH

SEQ ID NO: 18 - protein, 69 aa, Artificial Sequence
  Synthetic Sequence
IQCAHYIDGPHCVKTCPAGVMGENNTLVWKYADAGHVCHLCHPNCTYGCTGPGLEGCPTSAHHHHHHHH

SEQ ID NO: 19 - protein, 93 aa, Artificial Sequence
  Synthetic Sequence
IQCHPECLPQVMNITCTGRGPDNCIQCAHYIDGPHCVKTCPAGVMGENNTLVWKYADAGHVCHLCHPNCTYGCTGPGLEGCARSAHHHHHHHH

SEQ ID NO: 20 - protein, 69 aa, Artificial Sequence
  Synthetic Sequence
IQCAHYIDGPHCVKTCPAGVMGENNTLVWKYADAGHVCHLCHPNCTYGCTGPGLEGCARSAHHHHHHHH

SEQ ID NO: 21 - protein, 624 aa, Artificial Sequence
  Synthetic Sequence
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQSAHHHHHHHH

SEQ ID NO: 22 - protein, 623 aa, Artificial Sequence
  Synthetic Sequence
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTSAHHHHHHHH

SEQ ID NO: 23 - protein, 451 aa, Artificial Sequence
  Synthetic Sequence
QVQLKQSGPGLVQPSQSLSITCTVSGFSLTSYGVHWVRQSPGKGLEWLGVIWSGGTTDYNAAFISRLSISQDNSKSQVFFKMNSLQANDTAIYYCAGNQGRTVRPTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, Artificial Sequence
  Synthetic Sequence
DILLTQSPAILSVSPGERVSFSCRASQSIGTSIHWYQQRANGSPRLLIKYASESISGIPSRFSGSGSGTDFTLSINSVESEDIAHYYCQQNSNWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 450 aa, Artificial Sequence
  Synthetic Sequence
DVQLQESGPGRVKPSQSLSLTCTVTGYSLTSDYAWSWIRQFPGNRLEWMVYITYSGITTYNPSLKSRISITRDTSKNQFFLQLNSVTTEDTATYYCAGGDYGLSSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence
  Synthetic Sequence
DIVLTQSPVTLSVTPGDRVSLSCRASQSISNYLHWYQQKSHESPRLLIKYASQSISGIPSRFSGSGSGTDFTLSINSVETEDFGMYFCQQSNNWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 97 aa, Artificial Sequence
  Synthetic Sequence
QVQLQESGPGLVKPSETLSLTCTVSGGSISSYYWSWIRQPPGKGLEWIGYIYTSGSTNYNPSLKSRVTISVDTSKNQFSLKLSSVTAADTAVYYCAR

SEQ ID NO: 28 - protein, 95 aa, Artificial Sequence
  Synthetic Sequence
EIVLTQSPDFQSVTPKEKVTITCRASQSIGSSLHWYQQKPDQSPKLLIKYASQSISGVPSRFSGSGSGTDFTLTINSLEAEDAATYYCHQSSSLP

SEQ ID NO: 29 - protein, 104 aa, Artificial Sequence
  Synthetic Sequence
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGPYNYL

SEQ ID NO: 30 - protein, 95 aa, Artificial Sequence
  Synthetic Sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 31 - protein, 121 aa, Artificial Sequence
  Synthetic Sequence
QVQLQESGPGLVKPSETLSLTCTVSGFSLTSYGVHWVRQPPGKGLEWLGVIWSGGTTDYNAAFISRLTISQDNSKNQVSLKLSSVTAADTAVYYCAGNQGRTVRPTWFAYWGQGTTVTVSS

SEQ ID NO: 32 - protein, 121 aa, Artificial Sequence
  Synthetic Sequence
EVQLVESGGGLVQPGGSLRLSCAVSGFSLTSYGVHWVRQAPGKGLEWLGVIWSGGTTDYNAAFISRLTISQDNSKNTVYLQMNSLRAEDTAVYYCAGNQGRTVRPTWFAYWGQGTMVTVSS

SEQ ID NO: 33 - protein, 107 aa, Artificial Sequence
  Synthetic Sequence
EIVLTQSPDFQSVTPKEKVTITCRASQSIGTSIHWYQQKPDQSPKLLIKYASESISGVPSRFSGSGSGTDFTLTINSLEAEDAATYYCQQNSNWPLTFGQGTKLEIK

SEQ ID NO: 34 - protein, 107 aa, Artificial Sequence
  Synthetic Sequence
DIQLTQSPSSLSASVGDRVTITCRASQSIGTSIHWYQQKPGKAPKLLIKYASESISGVPSRFSGSGSGTDFTLTISSLQPEDFATYYCQQNSNWPLTFGQGTKVEIK

SEQ ID NO: 35 - protein, 99 aa, Artificial Sequence
  Synthetic Sequence
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 36 - protein, 120 aa, Artificial Sequence
  Synthetic Sequence
QVQLQESGPGLVKPSQTLSLTCTVSGYSLTSDYAWSWIRQPPGKGLEWMVYITYSGITTYNPSLKSRITISRDTSKNQFSLKLSSVTAADTAVYYCAGGDYGLSSWFAYWGQGTTVTVSS

SEQ ID NO: 37 - protein, 120 aa, Artificial Sequence
  Synthetic Sequence
EVQLVESGGGLVQPGGSLRLSCAVSGYSLTSDYAWSWIRQAPGKGLEWMVYITYSGITTYNPSLKSRITISRDTSKNTFYLQMNSLRAEDTAVYYCAGGDYGLSSWFAYWGQGTMVTVSS

SEQ ID NO: 38 - protein, 107 aa, Artificial Sequence
  Synthetic Sequence
EIVLTQSPDFQSVTPKEKVTITCRASQSISNYLHWYQQKPDQSPKLLIKYASQSISGVPSRFSGSGSGTDFTLTINSLEAEDAATYFCQQSNNWPYTFGQGTKLEIK

SEQ ID NO: 39 - protein, 107 aa, Artificial Sequence
  Synthetic Sequence
DIQLTQSPSSLSASVGDRVTITCRASQSISNYLHWYQQKPGKAPKLLIKYASQSISGVPSRFSGSGSGTDFTLTISSLQPEDFATYFCQQSNNWPYTFGQGTKVEIK

SEQ ID NO: 40 - protein, 451 aa, Artificial Sequence
  Synthetic Sequence
QVQLQESGPGLVKPSETLSLTCTVSGFSLTSYGVHWVRQPPGKGLEWLGVIWSGGTTDYNAAFISRLTISQDNSKNQVSLKLSSVTAADTAVYYCAGNQGRTVRPTWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, Artificial Sequence
  Synthetic Sequence
EIVLTQSPDFQSVTPKEKVTITCRASQSIGTSIHWYQQKPDQSPKLLIKYASESISGVPSRFSGSGSGTDFTLTINSLEAEDAATYYCQQNSNWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 451 aa, Artificial Sequence
  SEQ ID NO 0423A6-stable antibody heavy chain
EVQLVESGGGLVQPGGSLRLSCAVSGFSLTSYGVHWVRQAPGKGLEWLGVIWSGGTTDYNAAFISRLTISQDNSKNTVYLQMNSLRAEDTAVYYCAGNQGRTVRPTWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 214 aa, Artificial Sequence
  Synthetic Sequence
DIQLTQSPSSLSASVGDRVTITCRASQSIGTSIHWYQQKPGKAPKLLIKYASESISGVPSRFSGSGSGTDFTLTISSLQPEDFATYYCQQNSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 450 aa, Artificial Sequence
  Synthetic Sequence
QVQLQESGPGLVKPSQTLSLTCTVSGYSLTSDYAWSWIRQPPGKGLEWMVYITYSGITTYNPSLKSRITISRDTSKNQFSLKLSSVTAADTAVYYCAGGDYGLSSWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 214 aa, Artificial Sequence
  Synthetic Sequence
EIVLTQSPDFQSVTPKEKVTITCRASQSISNYLHWYQQKPDQSPKLLIKYASQSISGVPSRFSGSGSGTDFTLTINSLEAEDAATYFCQQSNNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 450 aa, Artificial Sequence
  Synthetic Sequence
EVQLVESGGGLVQPGGSLRLSCAVSGYSLTSDYAWSWIRQAPGKGLEWMVYITYSGITTYNPSLKSRITISRDTSKNTFYLQMNSLRAEDTAVYYCAGGDYGLSSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 214 aa, Artificial Sequence
  Synthetic Sequence
DIQLTQSPSSLSASVGDRVTITCRASQSISNYLHWYQQKPGKAPKLLIKYASQSISGVPSRFSGSGSGTDFTLTISSLQPEDFATYFCQQSNNWPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 451 aa, Artificial Sequence
  Synthetic Sequence
QVQLKQSGPGLVQPSQSLSITCTVSGFSLTSYGVHWVRQSPGKGLEWLGVIWSGGTTDYNAAFISRLSISQDNSKSQVFFKMNSLQANDTAIYYCAGNQGRTVRPTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 49 - protein, 482 aa, Artificial Sequence
  Synthetic Sequence
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESDGSFALSSRLRVDKSRWQQGNVFSCSVMHEALHNHYTQKSLSLSPGK

SEQ ID NO: 50 - protein, 450 aa, Artificial Sequence
  Synthetic Sequence
DVQLQESGPGRVKPSQSLSLTCTVTGYSLTSDYAWSWIRQFPGNRLEWMVYITYSGITTYNPSLKSRISITRDTSKNQFFLQLNSVTTEDTATYYCAGGDYGLSSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 51 - protein, 451 aa, Artificial Sequence
  Synthetic Sequence
QVQLQESGPGLVKPSETLSLTCTVSGFSLTSYGVHWVRQPPGKGLEWLGVIWSGGTTDYNAAFISRLTISQDNSKNQVSLKLSSVTAADTAVYYCAGNQGRTVRPTWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 52 - protein, 450 aa, Artificial Sequence
  Synthetic Sequence
QVQLQESGPGLVKPSQTLSLTCTVSGYSLTSDYAWSWIRQPPGKGLEWMVYITYSGITTYNPSLKSRITISRDTSKNQFSLKLSSVTAADTAVYYCAGGDYGLSSWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK